(19) United States
(12) Patent Application Publication
(10) Pub. No.: US 2026/0209356 A1
(43) Pub. Date: Jul. 23, 2026

(54) IGG DERIVED B7-H3-SPECIFIC CHIMERIC ANTIGEN RECEPTOR (B7-H3 CAR) EFFECTOR CELLS FOR THE TREATMENT OF CD276+ TUMORS AND AUTOIMMUNE DISEASES

(71) Applicant: Ospedale Pediatrico Bambino Gesù, Rome (IT)

(72) Inventors: Franco LOCATELLI, Rome (IT); Concetta QUINTARELLI, Rome (IT); Biagio DE ANGELIS, Rome (IT); Ignazio CARUANA, Rome (IT); Lorenzo MORETTA, Rome (IT); Cristina BOTTINO, Genoa (IT); Roberta CASTRICONI, Genoa (IT)

(21) Appl. No.: 19/139,433

(22) PCT Filed: Dec. 13, 2023
(86) PCT No.: PCT/IT2023/050280

§ 371 Date: Jun. 16, 2025

(30) Foreign Application Priority Data
Dec. 14, 2022 (IT) 102022000025596

(51) Int. Cl. C07K 16/28; A61K 38/00; A61K 40/11; A61K 40/31; A61K 40/42; A61P 35/02; C07K 14/705; C12N 5/0783; C12N 9/64; C12N 15/86

(52) U.S. Cl. CPC C07K 16/2827; A61K 40/11; A61K 40/31; A61K 40/421; A61P 35/02; C07K 14/7051; C12N 5/0636; C12N 9/6472; C12N 15/86; C12Y 304/22062; A61K 38/00; C07K 2319/33; C12N 2501/2307; C12N 2501/2315; C12N 2510/00; C12N 2740/10043; C12N 2840/20

(57) ABSTRACT

The present invention concerns a vector including the cassette coding for B7-H3 CAR gene obtained using the single chains variable fragments (scFv) of the monoclonal IgG antibody NE97, a method for the production thereof and B7-H3 CAR genetically modified effector cells (such as T cells or innate cells such as NK and NK-T cells) for the treatment of CD276 (B7-H3) positive tumors such as lymphoid malignancies, leukemia and solid tumors such as CNS tumors, extra-cranial and intracranial tumors and autoimmune diseases.

The present invention concerns IgG derived B7-H3-Specific Chimeric Antigen Receptor (B7-H3 CAR) effector cells for the treatment of B7-H3 (CD276) positive tumors and autoimmune diseases. In particular, the present invention concerns a vector including the cassette coding for B7-H3 CAR gene obtained using the single chains variable fragments (scFv) of the monoclonal IgG antibody NE97, a method for the production thereof and B7-H3 CAR genetically modified effector cells (such as T cells or innate cells such as NK and NK-T cells) for the treatment of CD276 (B7-H3) positive tumors such as lymphoid malignancies, leukemia and solid tumors such as CNS tumors, extra-cranial and intracranial tumors and autoimmune diseases.

B7-H3 [B7-homolog 3 or CD276] is a type I transmembrane protein encoded by chromosome 15 in humans (PMID: 27208063).

B7-H3 is both an inhibitory ligand for natural killer cells and T cells and a tumor antigen widely expressed among human solid tumours (PMID 26487718).

Normal human tissue microarrays showed that the brain, lungs, and heart were B7-H3-negative, while the bladder, skin, and rectum exhibited weak cytoplasmic staining in the epithelium or stroma. The stomach, colon, liver, pancreas, testis and prostate showed positive staining, but the staining intensity was much lower than that in solid tumours. (PMID: 33811153).

Despite broad mRNA expression in such non-lymphoid and lymphoid organs as the liver, heart, prostate, spleen, and thymus, the protein expression is limited at steady state, suggesting the presence of an important posttranscriptional control mechanism (PMID: 27208063). B7-H3, which belongs to the B7/CD28 superfamily, is constitutively found at low level also on non-immune resting cell types, fibroblasts, endothelial cells (EC), osteoblasts, and amniotic fluid stem cells. Moreover, B7-H3 expression is induced on immune cells, specifically APCs (PMID: 17615586). In addition, B7-H3 can also detected on natural killer (NK) cells, B cells, and a minor population of T cells following PMA/ionomycin stimulation. (PMID: 14764704).

B7-H3 is found on the membrane, in the cytoplasm, or within the nucleus of cancer cells, but also on the tumor-associated vasculature (PMID: 22473715).

Recently, several studies have demonstrated aberrant overexpression of B7-H3 on tumor cells and cancer-associated stromal cells in both solid and hematologic malignancies including, leukaemia. Specifically, B7-H3 protein has been found overexpressed in acute myeloid leukemia (PMID: 26376842), prostate cancer (PMID: 17686830), breast cancers (PMID: 21107115) (PMID: 29344150), melanoma (PMID: 21671471), colorectal cancer (PMID: 31466914) (PMID: 22473715), ovarian cancer (PMID: 28765941) neuroblastoma (PMID: 15314238), osteosarcoma (PMID: 23940627), and Central Nervous System (CNS) tumors (PMID: 31887631). In pediatric CNS tumors, high B7-H3 expression has been found in the following tumors: atypical teratoid/rhabdoid tumors (ATRT), ependymomas (all grades), medulloblastoma (MB), CNS embryonal tumors, choroid plexus tumors (CPTs), meningioma and craniopharyngiomas (PMID: 31887631) (PMID: 30027617). In MB, the highest B7-H3 expression observed in the WNT subtype, while the lowest in the SHH subtype, while low-grade gliomas and germ cell tumors did not differ from the normal brain in their levels of B7-H3 expression (PMID: 31887631). Interestingly, in neuroblastoma primary tumours, B7-H3 expression was detected also on tumor cells lacking the G2D tumor associated marker, making then un-responsive to the currently used immunotherapeutic protocol based on the infusion of an anti-G2D mAb (PMID: 33795387). Moreover, the expression of B7-H3 was upregulated by IFN-γ in neuroblastoma cells, particularly when cultured in 3D condition (PMID: 31447858, 35205760).

B7-H3-specific monoclonal antibodies (mAbs) and antibody-drug conjugates showed antitumor activity against B7-H3+ tumor cells in xenograft mouse models, and several phase I clinical trials showed a good safety profile (PMID: 33051306.)

A phase I clinical trial with [¹²⁴I]-8H9 radio-immunotherapy agent targeting B7-H3 antigen in children with diffuse intrinsic pontine glioma (clinical trial NCT01502917) (PMID: 30102232) showed to be safe and, more important, the maximum-tolerated dose was not reached, and no dose-limiting toxicities occurred. Due to its broad expression across multiple tumor types and preliminary clinical results, B7-H3 can be considered an attractive target for cancer immunotherapy. Immunotherapeutic approaches targeting B7-H3 by CAR has been demonstrated of value in preclinical solid tumors and brain tumors models (PMID: 30655315) (PMID: 32341579) (PMID: 31466914) (PMID: 30753824) (PMID: 33653946), in acute leukemia (PMID: 33531429) and Anaplastic Large Cell lymphomas (PMID: 33348781).

Several clinical trials are ongoing (ClinicalTrials.gov Identifier: NCT04385173; NCT04077866; NCT04185038, etc.), but no results have been published so far.

Table 1 summarizes 1B7-1H3 CAR T-cells characterized in preclinical models (Table 1). The symbols used in this table are mAb: monoclonal antibodies; ScFv: single chain variable fragment. GFP: Green Fluorescence Protein; mCherry is a member of the mFruits family of monomeric red fluorescent proteins (mRFPs). tdTomato is a basic (constitutively fluorescent) orange fluorescent protein.

TABLE 1

| | CAR | | | | | | |
|---|---|---|---|---|---|---|---|
| | Gene | | trackable | | | Costimulatory | Reference |
| Platform | ration | Single chain | marker | Hinge | TM | domains | PMID |
| Retrovirus | 2 | MGA271 | CH2-CH3 | CH2-CH3 | CD8a | 4-1BB | 30655315 |
| | | humanized | | | | | |
| | | mAb | | | | | |
| Retrovirus | 2 | 376.96 | GFP | CD8a | CD8a | 4-1BB | 30753824 |
| Retrovirus | 2 | murine | GFP | CD8a | CD8a | CD28 | 30753824 |
| | | mAb | | | | | |
| Lentivirus | 3 | 8H9 | mCherry | CD8a | CD8a | CD28.4-1BB | 31485480 |
| | | humanized | | | | | |
| | | mAb | | | | | |
| Lentivirus | 3 | 8H9 | GFP | CD8a | CD8a | CD28.4-1BB | 33811153 |
| | | humanized | | | | | |
| | | mAb | | | | | |
| Lentivirus | 3 | J42 | mCherry | CD8a | CD8a | CD28.4-1BB | 32346608 |
| | | murine | | | | | |
| | | mAb | | | | | |
| Lentivirus | 2 | CD70 mAb/ | tdTomato | CD8a | CD8a | 4-1BB | 32685008 |
| | | B7H3 (J42 | | | | | |
| | | clone murine) | | | | | |
| | | mAb | | | | | |
| Lentivirus | 2 | MGA271 | ScFv | CD8a | CD8a | CD28 | 32728609 |
| Lentivirus | | humanized | | CD8a | CD8a | 4-1BB | |
| Lentivirus | | mAb | | CD28 | CD28 | CD28 | |
| Lentivirus | | | | CD28 | CD28 | 4-1BB | |

Second-generation B7-H3 CAR T-cells, whose binder is derived from a humanized antibody (Ab) (MGA271, enoblituzumab) were developed and published in 2019 (PMID: 30655315). The B7-H3 single chain variable fragment (scFv) was introduced into an MSGV.1 retroviral expression vector containing a CD8-a hinge-transmembrane domain, a CD137 (4-1BB) costimulatory motif, and a CD3z signaling domain (second generation of CAR). As trackable marker, the sequence of the human IgG1 constant domain (CH2-CH3) was inserted between the scFv and the transmembrane (TM) domains. Generated B7-H3 CAR T cells were growth in media containing interleukin 2 (IL2). Preclinical studies demonstrated the ability of these cells to lyse orthotopic models of osteosarcoma (MG63.3 cell line), Ewing sarcoma (EW8 cell line), MB (DAOY and D425 cell line) and Atypical teratoid/rhabdoid tumors (ATRTs) xenograft (PMID: 32341579).

In the same year (2019) it was reported preclinical result about a different second-generation B7-H3 CAR T-cells (B7-H3.CAR-Ts), using a single-chain variable fragment (scFv) derived from the B7-H3 376.96 mAb, (PMID: 24216048) (PMID: 6951087) (PMID: 28104527) and including either CD28 or 4-1BB endodomains (B7-H3.CAR-28ζ and B7-H3.CAR-BBC, respectively). Second-generation B7-H3.CAR-Ts contained central-memory, effector-memory, and T stem cell memory, without significant differences between CD28 and 4-1BB co-stimulation. Preclinical studies demonstrated the ability of CAR-T cells to lyse pancreatic ductal adenocarcinoma (PDAC), ovarian cancer (OC) and neuroblastoma in vitro and in orthotopic and metastatic xenograft mouse models, which included patient-derived xenograft (PMID 30753824). Moreover, they showed that 4-1BB costimulatory domain induces lower PD1 expression in B7-H3.CAR-Ts compared with CD28 co-stimulation and thus better efficacy when targeting tumor cells expressing PD-L1. Moreover, both 376.96 B7-H3.CAR-Ts showed to be effective also against GBM cell lines and patient-derived GBM neurospheres in vitro and in xenograft murine models. No significant differences were found between CD28 and 4-11BB co-stimulation, although CD28-co-stimulated CAR-T cells released more inflammatory cytokines (PMID: 31466914). B7-H3.CAR-Ts exhibit efficient antigen-dependent cytotoxicity in vitro and in xenograft models of AML, and are unlikely to cause unacceptable hematopoietic toxicity. (PMID: 33531429)

In 2019, a third generation of B7-H3.CAR using a single-chain variable fragment (scFv) derived from the published humanized 8H9 mAb (PMID: 26487718) and including CD28 and 4-1BB endodomains has been proposed on lentiviral platform to treat Glioblastoma tumors (PMID: 31485480). The expression of the CARs on T cells after lentivirus transduction, a member of the mFruits family of monomeric red fluorescent proteins (mRFPs), spaced by a P2A self-cleaving peptide (PMID: 31485480).

In 2020, this construct was used to treat a 56-year-old patient, with recurrent glioblastoma (GBM), who was weekly intracavitary infused with B7-H3 targeted CAR-T cells. During cycles 1-5, the patient suffered from recurrent headache. After the first-round infusion, they observed a dramatic reduction of recurrent tumor by magnetic resonance imaging (MRI), and the clinical response was sustained for about 50 days. Unfortunately, altered consciousness in cycle 6 and 7 and MRI revealed tumor recurrence. Finally, the patient dropped out of the clinical study after the 7 cycles infusion. (PMID: 33767145)

In 2021, a similar B7-H3.CAR lentiviral vector was proposed including the same single-chain variable fragment (scFv) derived from the published humanized 8H9 mAb (PMID: 26487718), CD28 and 4-1BB as costimulatory endodomains and the Green Fluorescence Protein (GFP) as trackable marker (PMID: 33811153). Preclinical studies demonstrated the ability of these CAR-T cells to lyse two triple-negative breast cancer (TNBC) cell lines (SUM149 and TNBC-S3), two head and neck squamous cell carcinoma (HNSCC) cell lines (HNSCC-Y2 and SCC-9), one non-small cell lung carcinoma (NSCLC) cell line (A549), and one skin cutaneous melanoma SKCM cell line (M21).

A different B7-H3-redirected CAR was based on a lentiviral vector encoding the B7-H3 binder J42-scFv, CD28 and 4-1BB costimulatory domains, and the CD3-z signaling domain, mCherry was inserted as a tracker for detecting the expression of CAR with FACS. B7-H3-targeted CAR-T cells exhibit antitumor effects on hematologic tumors (B-myelomonocytic leukemia MV-4-11, histiocytic lymphoma U937), and solid tumors: human melanoma cell line A375, and hepatocellular carcinoma HepG2. (PMID: 32346608)

More recently, it has been published a second generation of lentiviral vector containing a TanCAR molecule consisting of a CD8 leader, followed by CD70 specific scFv that is separated from B7-H3 specific scFv (clone: mAb-J42) by a 15-amino acid glycine/serine repeat linker, hinge domain, CD8 transmembrane, the signaling domain of the costimulatory molecule 4-1BB, the signaling domain of the T cell receptor CD3-zeta chain. A P2A ribosome skip sequence separates the CAR sequence from a tdTomato as a CAR-T cell tracker. (PMID: 32685008). In preclinical model of human lung cancer and melanoma, this bivalent targeting CAR-T cells could not only induce a more superior antitumor effect but also induces regression of tumor in a lower dose than a single targeting CAR-T cells (PMID: 32685008).

Second lentiviral vectors B7-H3-CARs, utilizing a single-chain variable fragment (scFv) derived from the humanized B7-H3-specific monoclonal antibody (mAb) MGA271, with different hinge/transmembrane (CD8a versus CD28) and CD28 or 41BB costimulatory domains (CD8a/CD28, CD8a/41BB, CD28/CD28, CD28/41BB) has been evaluated in vitro and in vivo xenograft models. CD8a/CD28-CAR T cells consistently outperformed other CAR T cell populations in three animal models, resulting in a significant survival advantage. 41BBL expression on the surface of CD8a/CD28-CAR T cells enhanced their ability to kill tumor cells in repeat stimulation assays. (PMID: 32728609). CAR detection was performed using F(ab0)2 fragment Single chain variable Fragment B7-H3 CAR specific antibody (PMID: 32728609).

However, known B7-H3.CAR are characterized by the lack of a safety switch.

In addition, the trackable markers mostly used in known B7-H3.CARs, GFP, mCherry and tdTomato, are intracellular reporter molecules, to evaluate gene transfer and expression, which can be detected in living cells without selection or staining.

However, preclinical studies in animal in vivo models have shown that the GFP protein can induce the expansion of anti GFP T lymphocytes advice against its use in the clinic setting (PMID: 10455440)(PMID: 27435468).

In the light of the above, it is apparent the need to provide for further B7-H3.CAR T-cells, which are able to overcome all the disadvantages of the known B7-H3.CAR T-cells.

According to the present invention, novel B7H3-specific chimeric antigen receptors (B7-H3.CAR) of third generation are now provided.

In particular, bicistronic vectors have been designed, which allow the simultaneous expression of two transgenes, namely the inducible Caspase 9 (iC9) and the third generation B7-H3.CARs.

A clonal retroviral producer cell line has been generated that is able to produce high titer of retroviral vector containing:
a ΔCD34 flag, represented by the extracellular domain of human CD34 linked to the CD8 transmembrane portion, with a double function:
a) the selection of the genetically modified cells by clinical grade microbeads;
b) the phenotypic identification of the genetically modified cells.

The CAR construct was cloned after the gene cassette including the sequence of iC9 using a 2A sequence.

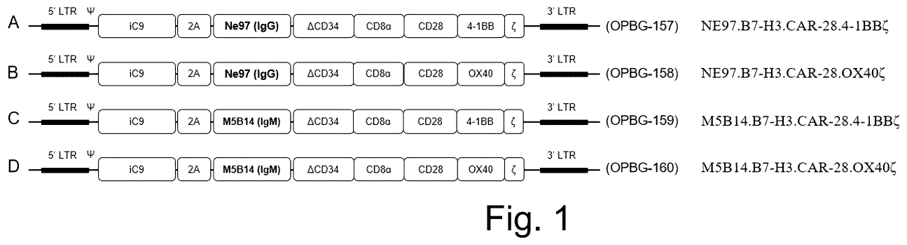

More specifically, the following clinical grade third generation of B7-H3.CAR SFG retroviral vectors have been prepared with the following functional and structural components (FIG. 1 A-D):
SFG.iC9-2A-(NE97)B7-H3.CAR-ΔCD34.CD8a.CD28.4-1BBζ (here after NE97.B7-H3.CAR-28.4-1BBζ)
SFG.iC9-2A-(NE97)B7-H3.CAR-ΔCD34.CD8a.CD28.OX40ζ (here after NE97.B7-H3.CAR-28.OX40ζ)
SFG.iC9-2A-(M5B14)B7-H3.CAR-ΔCD34.CD8a.CD28.4-1BBζ (here after M5B14.B7-H3.CAR-28.4-1BBζ)
SFG.iC9-2A-(M5B14)B7-H3.CAR-ΔCD34.CD8a.CD28.OX40ζ (here after M5B14.B7-H3.CAR-28.OX40ζ)

The vectors mentioned above comprise or consist of:
an inducible Caspase 9 (iC9) suicide gene as safety switch (PMID: 25389405; PMID: 29872565);
T2A self-cleaving peptides sequence, which can induce ribosomal skipping during translation of a protein in a cell (PMID: 28526819);
a signal peptide;
a single chain variable fragment (scFv) from (IgG) NE97 hybridoma, which was never applied for CAR therapy before; or an alternative single chain variable fragment (scFv) from (IgM) hybridoma, which was never applied for CAR therapy before;
a trackable marker CD34 derived epitope (ΔCD34) of only 16 amino acid (aa) (as trackable marker) for a rapid identification by FACS (Fluorescence-activated cell sorting) System of gene modified T cells;
an hinge represented by CD8 regions to avoid the immunogenic CH2-CH3 murine sequence (PMID: 25212991);
a transmembrane domain from the transmembrane domain of CD8 (CD8tm) to improve molecule stabilization;
a link domain (of only 7 aa) of CD8 cytoplasmic domain to improve molecule stabilization and connect CD8tm to costimulatory domains;
two costimulatory domains: CD28 (PMID: 17108138, PMID:19719389; PMID: 20944680; PMID: 26110267) and OX40 (PMID: 22754764; PMID: 23985696) or CD28 and 4-1BB (PMID: 19773745; PMID: 29872565) both fused respectively to CD3-ζ chain.

Table 2 shows the peculiar elements that are present in the B7-H3.CARs according to the present invention in comparison with known B7-H3.CAR, reported in table 1. The symbols used in this table are IgM: Immunoglobulin M; IgG: Immunoglobulin G; IC9: inducible Caspase 9; ΔCD34: CD34 derived epitope.

TABLE 2

| | CAR | | | | | Trans- | | |
|---|---|---|---|---|---|---|---|---|
| | Gene | Single | Suicide | trackable | | membrane | Costimulatory | Reference |
| Platform | ration | chain | Gene | marker | Hinge | (TM) | domains | PMID |
| Retroviral | 3 | M5B14 | iC9 | ΔCD34 | CD8 | CD8 | CD28.4-1BB | non- |
| | | (IgM) | | | | | | applicable |
| Retroviral | 3 | M5B14 | iC9 | ΔCD34 | CD8 | CD8 | CD28.OX40 | non- |
| | | (IgM) | | | | | | applicable |
| Retroviral | 3 | NE97 | iC9 | ΔCD34 | CD8 | CD8 | CD28.4-1BB | non- |
| | | (IgG) | | | | | | applicable |
| Retroviral | 3 | NE97 | iC9 | ΔCD34 | CD8 | CD8 | CD28.OX40 | non- |
| | | (IgG) | | | | | | applicable |

Therefore, the CAR molecules according to the present invention comprise two different single chains variable fragments (scFv): a fusion protein of the variable regions of the heavy (VH) and light (VL) chains of immunoglobulins, connected with a short linker peptide of ten to about 25 amino acids. The two scFv were derived from the monoclonal IgG antibody NE97 or from the monoclonal IgM antibody M51B14, respectively.

The scFvare cloned in frame with CD8 transmembrane domain, CD28 4.1BB or CD28.OX40 costimulatory domains, and CD3 zeta (CD3) cytoplasmic domain for the transduction of the activator signal after antigen engagement.

The clinical grade CAR construct was cloned in a retroviral vector after the gene cassette including the sequence of iC9 through the use of a 2A sequence.

Table 3 shows the functional elements of NE97.B7-H3.CARs vectors.

TABLE 3

| OPBG-157 retroviral Plasmid: | | | OPBG-158 retroviral Plasmid | | |
|---|---|---|---|---|---|
| NE97.B7-H3.CAR-28.4-1BBζ | | | NE97.B7-H3.CAR-28.OX40ζ | | |
| Component | Start | End | Component | Start | End |
| 5′-LTR | 9240 | 592 | 5′-LTR | 9222 | 592 |
| iC9 | 1880 | 3085 | iC9 | 1880 | 3085 |
| 2A sequence | 3086 | 3145 | 2A sequence | 3086 | 3145 |
| Signal Peptide | 3146 | 3208 | Signal Peptide | 3146 | 3208 |
| ScFv [VL) | 3209 | 3532 | ScFv [VL) | 3209 | 3532 |
| IgG NE97 | | | IgG NE97 | | |
| Flex | 3533 | 3556 | Flex | 3533 | 3556 |
| ScFv [VH] NE97 | 3557 | 3907 | ScFv [VH] NE97 | 3557 | 3907 |
| ΔCD34 | 3914 | 3961 | ΔCD34 | 3914 | 3961 |
| CD8 stalk (spacer) | 3962 | 4081 | CD8 stalk (spacer) | 3962 | 4081 |
| CD8TM | 4082 | 4150 | CD8TM | 4082 | 4150 |
| CD8 Cyt | 4151 | 4171 | CD8 Cyt | 4151 | 4171 |
| CD28 | 4178 | 4300 | CD28 | 4178 | 4300 |
| 14-1BB | 4301 | 4426 | OX40 | 4301 | 4408 |
| costimulation | | | costimulation | | |
| CD3z | 4427 | 4765 | CD3z | 4409 | 4747 |
| 3′ LTR | 5046 | 5515 | 3′ LTR | 5028 | 5497 |
| Amp^{R} | 6810 | 7670 | Amp^{R} | 6792 | 7652 |

For example, the functional and structural components for the expression and activity of SFG.iC9-2A-(NE97)B7-H3.CAR-ΔCD34.CD8a.CD28.4-1BBζ (here after NE97.B7-H3.CAR-28.4-1BBζ) are the following:
5′ LTR—Retroviral long terminal repeat at 5′ end of vector (functions as promoter sequence);
ψ—Retroviral encapsidation signal (psi; for packaging of RNA into virion particles);
iC9—Inducible Caspase 9 (iC9 gene contains the intracellular portion of the human caspase 9 protein, a pro-apoptotic molecule, fused to a drug-binding domain derived from human FK506-binding protein (FKBP12.caspase9, (iC9));
2A—encodes a synthetic 18 amino acid peptide from Thosea Asigna insect virus, which functions as a cleavable linker between the iC9 protein and CAR proteins;
Signal peptide—short amino acid sequence to allow the correct translocation of the secretory proteins from the Endoplasmic Reticulum to the cellular membrane;
A single chain variable fragment (ScFv) from NE97 hybridoma;
A trackable marker CD34 derived epitope (ΔCD34) of only 16 amino acid (aa) (as trackable marker) for a rapid identification by FACS (Fluorescence-activated cell sorting) System and/or selection by Cell Sorter System of gene modified cells;
A spacer represented by CD8 regions to avoid the immunogenic IgG4 Fc region;
a transmembrane domain from the transmembrane domain of CD8 (CD8TM) to improve molecule stabilization;
A small portion of CD8 cytoplasmatic portion between the CD8TM and intracellular domains: CD28.4-1BB-CD3 ζ chain (4.1BB-ζ);
3′ LTR—Retroviral long terminal repeat at 3′ end of vector (functions as terminator/polyadenylation sequences).

Table 4 shows the functional elements of M51B14.1B7-1H3.CARs vectors.

TABLE 4

| OPBG-159 retroviral Plasmid: | | | OPBG-160 retroviral Plasmid: | | |
|---|---|---|---|---|---|
| M5B14.B7-H3.CAR-28.4-1BBζ | | | M5B14.B7-H3.CAR-28.OX40ζ | | |
| Component | Start | End | Component | Start | End |
| 5′-LTR | 9240 | 592 | 5′-LTR | 9222 | 592 |
| iC9 | 1880 | 3085 | iC9 | 1880 | 3085 |
| 2A sequence | 3086 | 3145 | 2A sequence | 3086 | 3145 |
| Signal Peptide | 3146 | 3208 | Signal Peptide | 3146 | 3208 |
| ScFv [VL) | 3209 | 3532 | ScFv [VL) | 3209 | 3532 |
| IgM M5B14 | | | IgM M5B14 | | |
| Flex | 3533 | 3556 | Flex | 3533 | 3556 |
| ScFv [VH] NE97 | 3557 | 3907 | ScFv [VH] NE97 | 3557 | 3907 |
| ΔCD34 | 3914 | 3961 | ΔCD34 | 3914 | 3961 |
| CD8 stalk (spacer) | 3962 | 4081 | CD8 stalk (spacer) | 3962 | 4081 |
| CD8TM | 4082 | 4150 | CD8TM | 4082 | 4150 |
| CD8 Cyt | 4151 | 4174 | CD8 Cyt | 4151 | 4174 |
| CD28 | 4178 | 4300 | CD28 | 4178 | 4300 |
| 4-1BB | 4301 | 4426 | OX40 | 4301 | 4408 |
| costimulation | | | costimulation | | |
| CD3z | 4427 | 4765 | CD3z | 4409 | 4747 |
| 3′ LTR | 5046 | 5515 | 3′ LTR | 5028 | 5497 |
| Amp^{R} | 6810 | 7670 | Amp^{R} | 6792 | 7652 |

The above mentioned sequence according to the present invention provides unexpected advantages in comparison to known B7-H3.CARs.

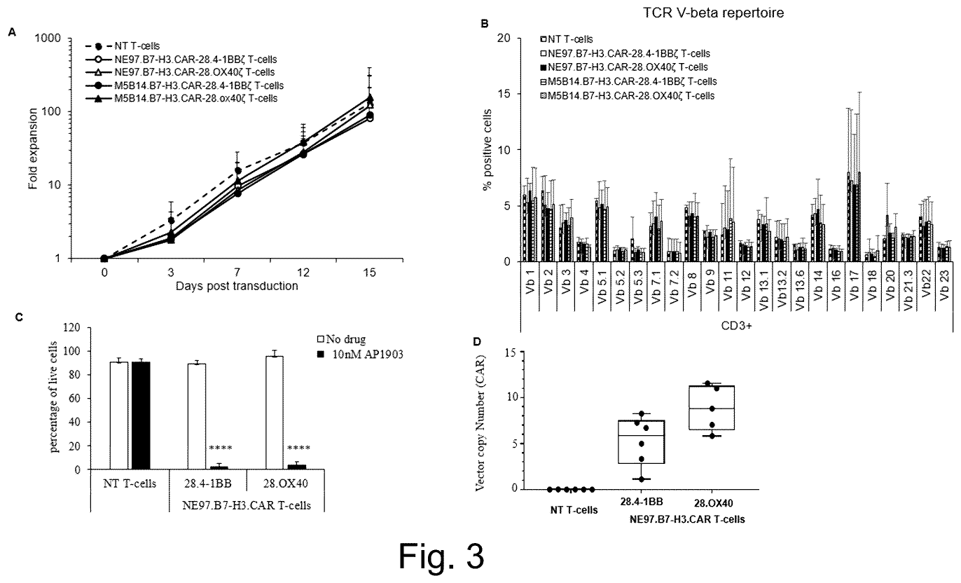

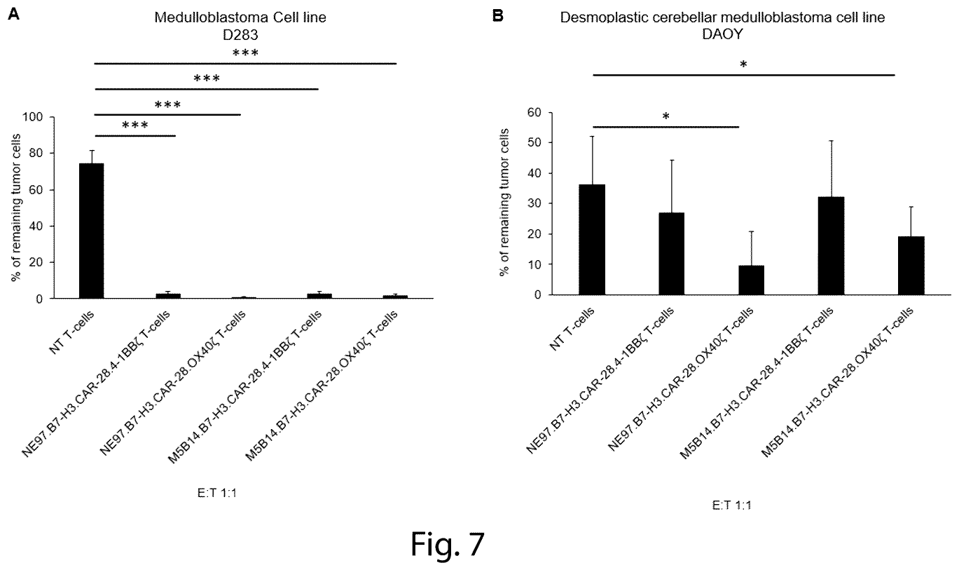

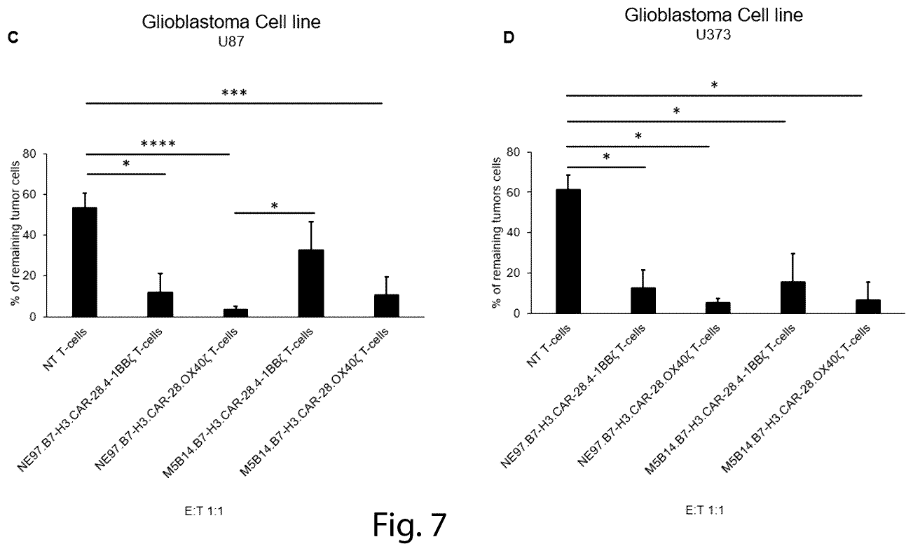

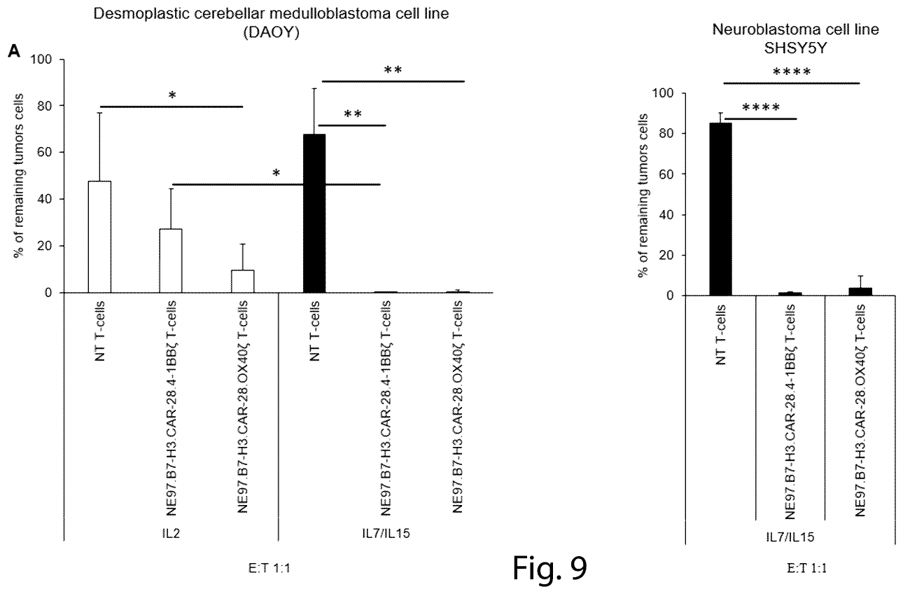

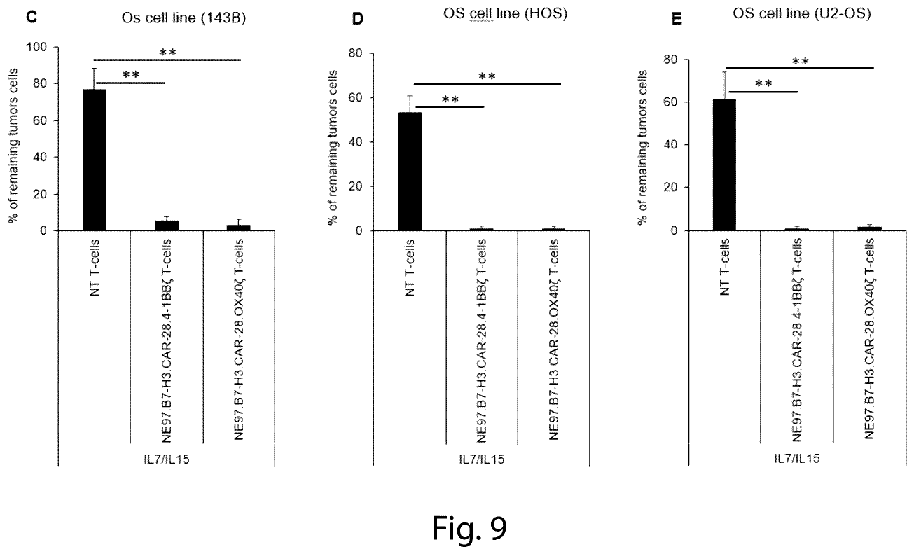

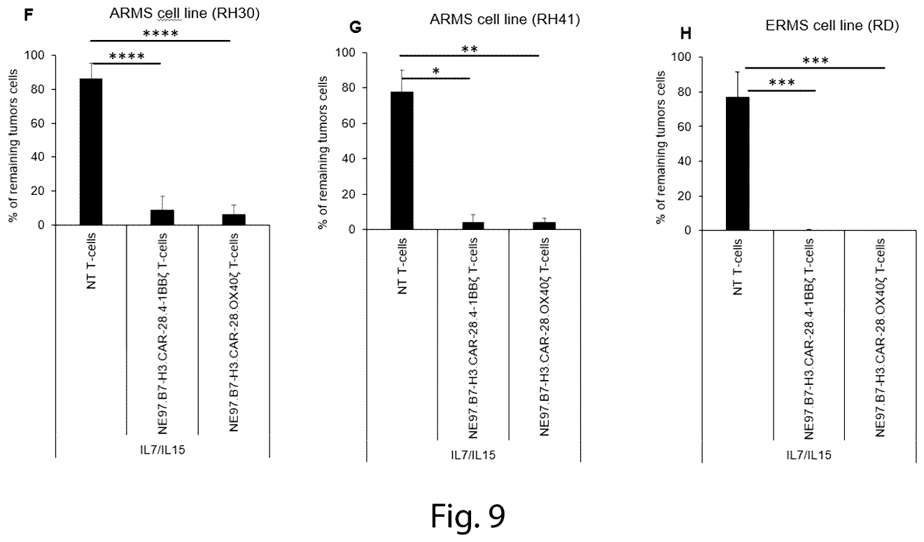

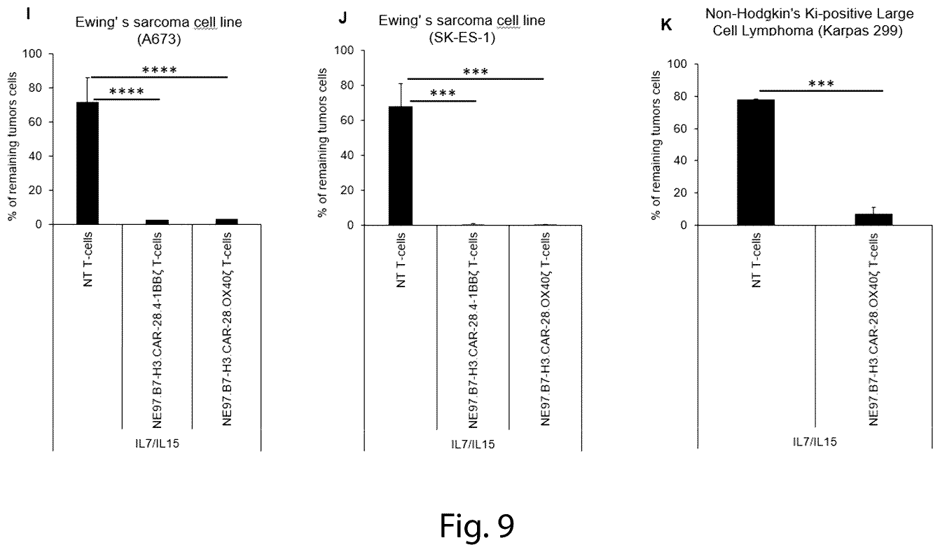

Xin Tang et al. recently reported a third generation CAR T Anti-B7-H3 (containing CD28 and 4-1BB as a costimulatory domain) capable of killing only tumor cell lines with high expression of B7-H3 (A172), failing to effectively kill the line of glioblastoma U87, characterized by a low expression of B7-H3 (PMID: 31485480: FIG. 3F page 283). In contrast, all four ^{III}B7-H3.CAR T cells (growth in IL2) according to the present invention exert a significant tumor control of glioblastoma cell line U87 (FIG. 7C). Furthermore, the experimental results reported below show that cytokine used in culture influence in vitro potency of B7-H3.CAR T-cells. Indeed, as show for the killing activity against the Desmoplastic cerebellar medulloblastoma cell line DAOY, the killing efficacy of (NE97) B7-H3.CARs can further increased by replacing IL2 with the combination of IL7/IL15 for their expansion (FIGS. 7B and 9A). In addition, the experimental results reported below show that, in vivo MB graft model, circulating (NE97) B7-H3.CAR T-cells show a very stable expression of CAR expression up to day +45 (80.65%±4.65%).

Results have been compared with those obtained using (M5B14) B7-H3.CAR, including the scFv of the M5B14 IgM anti-B7-H3 mAb. (NE97) B7-H3.CAR (including the scFv NE97) shows advantages also in comparison with the (M5B14) B7-H3.CAR, in term of more efficient stable CAR expression on T cells, a longer in vivo persistence, and higher anti-tumor activities thanks to the affinity of the (NE97) scFv with the antigen.

Advantageous results have been obtained also thanks to the choice of the production methods, such as the use of IL-7/IL-15 instead of IL-2.

The inclusion of an iC9 suicide gene, as safety switch, improves the safety of Gene therapy medicinal products (GTMP). The in vitro and in vivo results herewith described show that modified polyclonal (NE97) B7-H3.CAR T cells according to the present invention were able to eliminate very efficiently, in long-term co-culture, B7-H3+ tumours. The biological products according to the present invention in xenograft in vivo models show to eliminate both paediatric and adult haematological solid tumors (including the tumor of SNC), the rhabdomyosarcoma tumor cells, colorectal tumor, the glioma brain tumor and to establish a long-term immunological memory. More in detail, the retroviral supernatants obtained by all SFG retroviral vector were able to transduce efficiently activated T cells, with very high level of transduction. The introduction in the construct of CD34 derived epitope as trackable marker let easily to track the genetically modified T cells (CD3+CD34+) in vitro and in vivo xenograft mouse models.

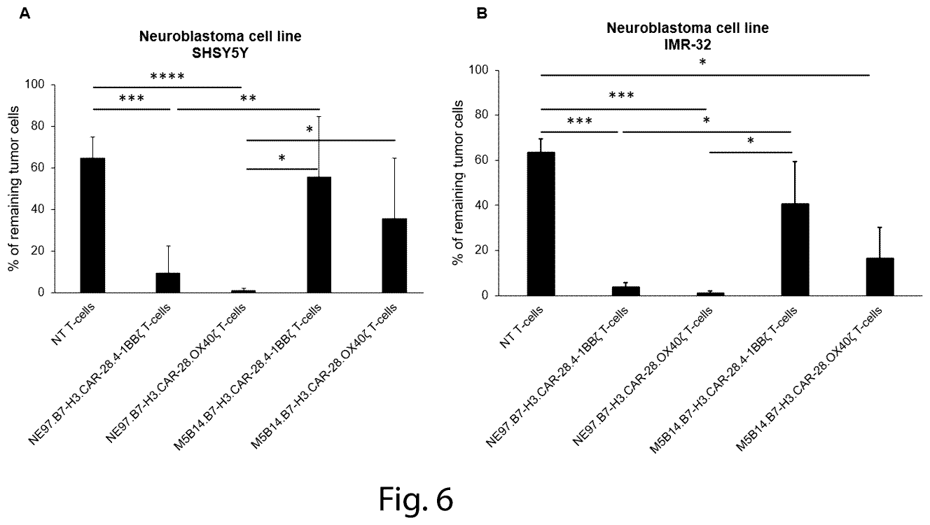

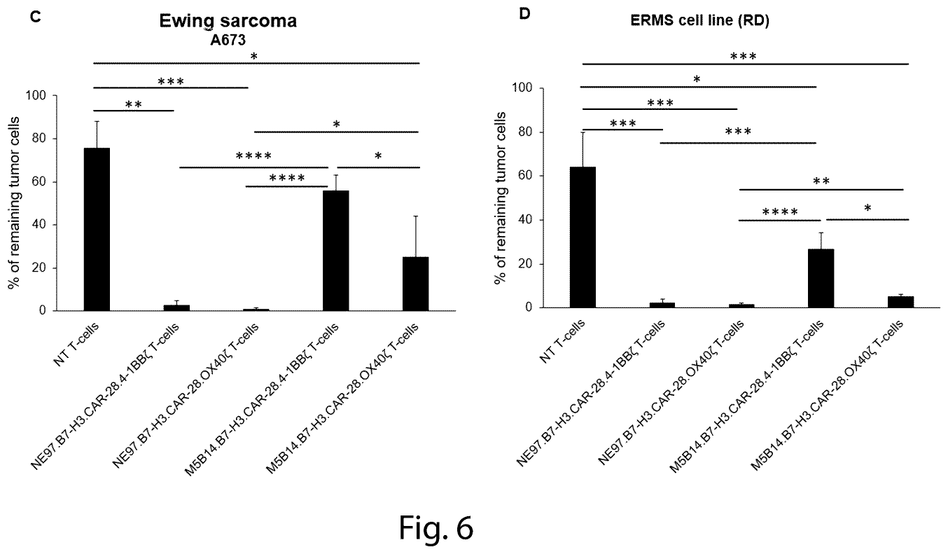

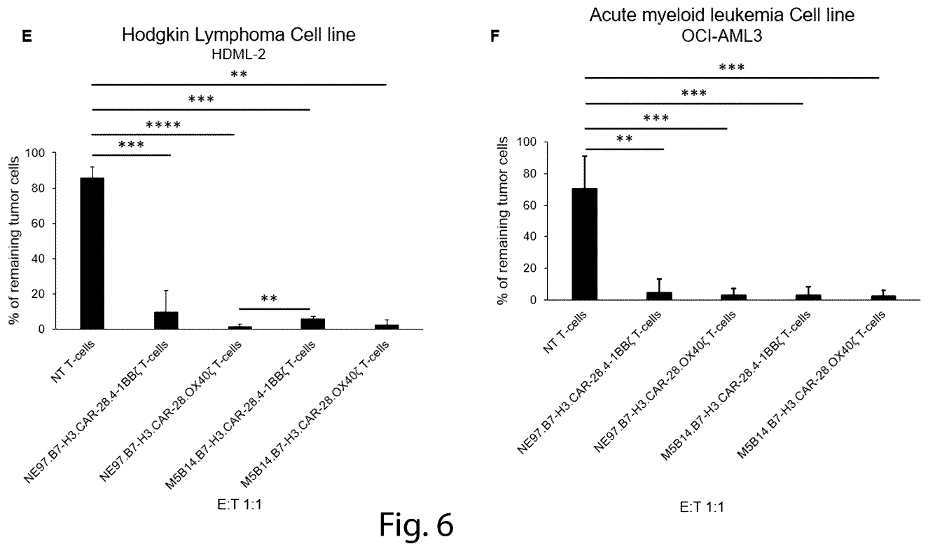

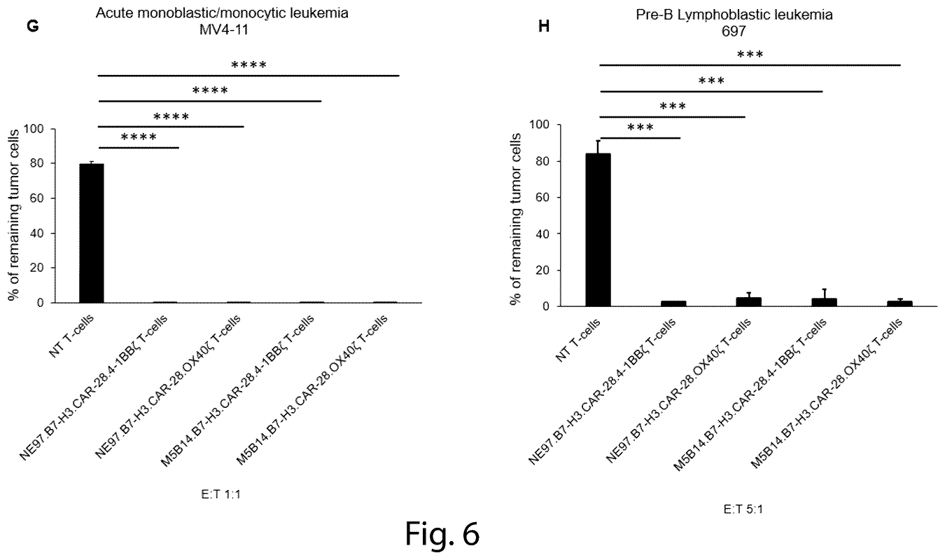

Globally, although NE97.B7-H3.CAR T-cells showed a superior anti-tumor activity against some solid tumors, such as the neuroblastoma cell lines SHSY5Y (FIG. 6A) and IMR-32 (FIG. 6B), the Ewing sarcoma A673 (FIG. 6C) and the ERMS cell line RD (FIG. 6D), compared to NT T-cells and M5B14.B7-H3.CAR T-cells, all four different clinical grade third generation of B7-H3.CAR T-cells according to the present invention are very active against B7-H3+leukemias/lymphomas cell lines and solid tumor cell lines. The experimental results described below show this activity against B7-H3+leukemias/lymphomas cell lines such as the Hodgkin lymphoma cell line HDML-2 (FIG. 6E), the acute myeloid leukemia cell line OCI-AML3 (FIG. 6F), the Acute monoblastic/monocytic leukemia cell line MV4-11 (FIG. 6G), the Pre-B lymphoblastic leukemia cell line 697 (FIG. 6H), and against solid tumor cell lines such as the medulloblastoma cell line D283 (FIG. 7A) and DAOY (FIG. 7B), and the Glioblastoma cell line U87 (FIG. 7C) and U373 (FIG. 7D).

It is also important to note that known B7-H3.CAR vector reported to date are second or third generation CARs: the second generation CARs preferentially include the CD28 (PMID:30753824; PMID:32728609) or 4-1BB domain as costimulatory domain (PMID:30655315; PMID:30753824; PMID:32685008; PMID:32728609), whereas third generations B7-H3.CARs vector (PMID:31485480; 3381153; PMID: 32346608) preferentially include the CD28-4 and 1 BB costimulatory domains.

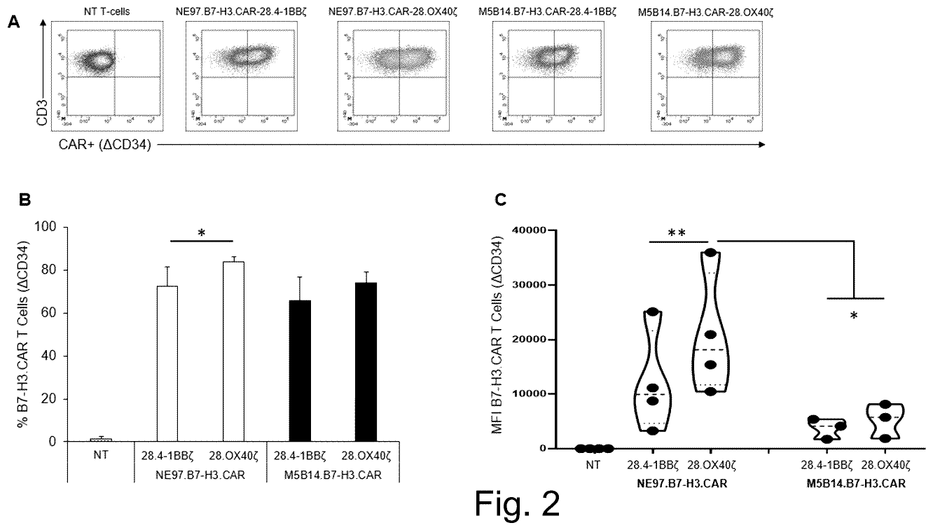

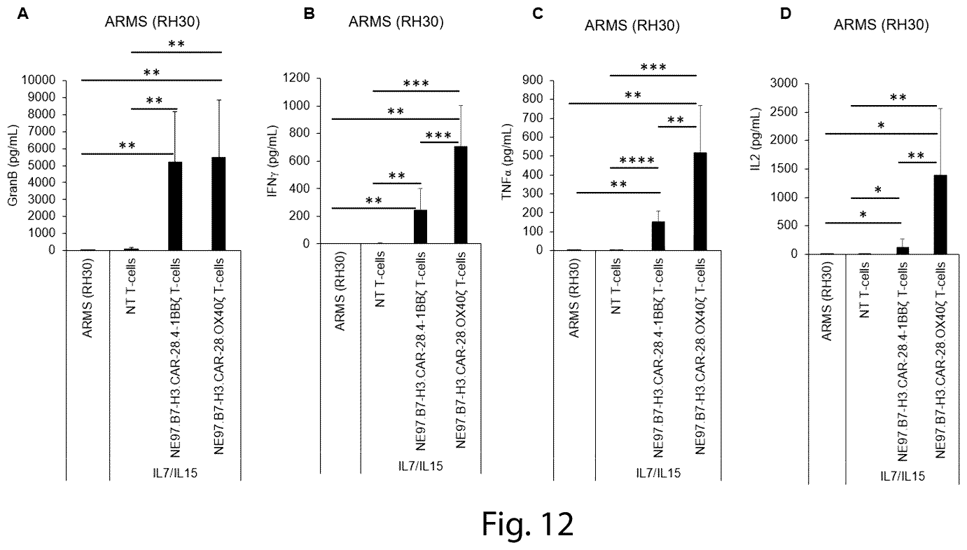

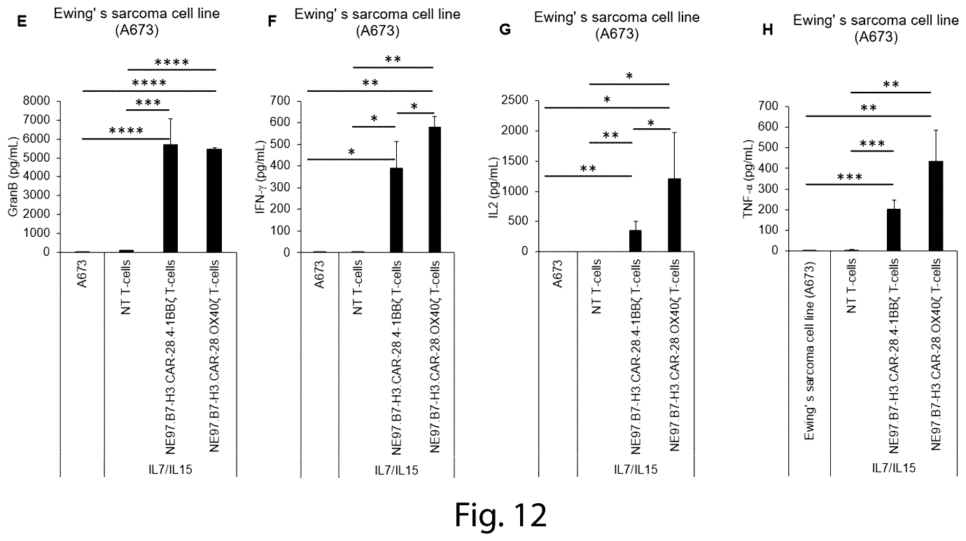

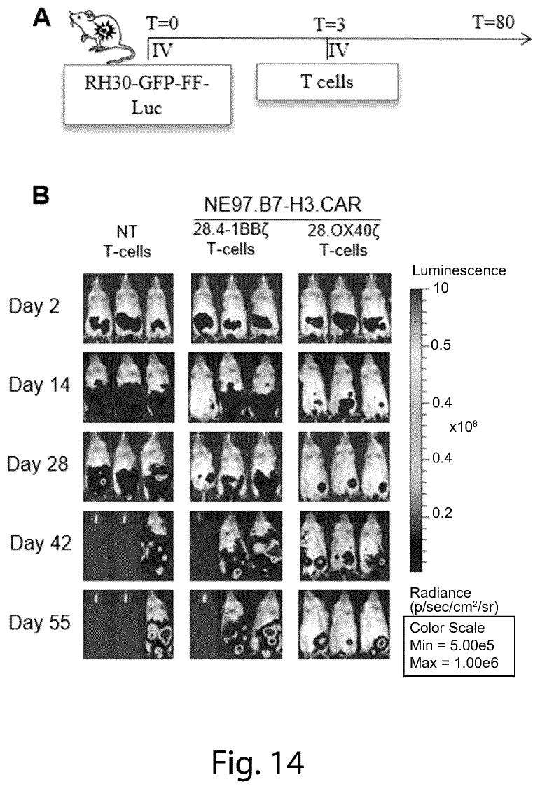

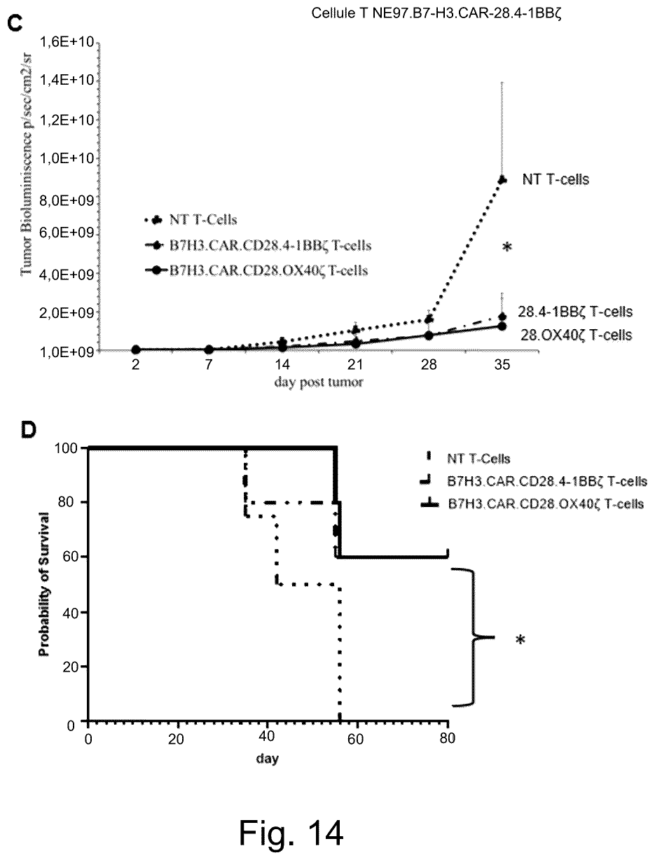

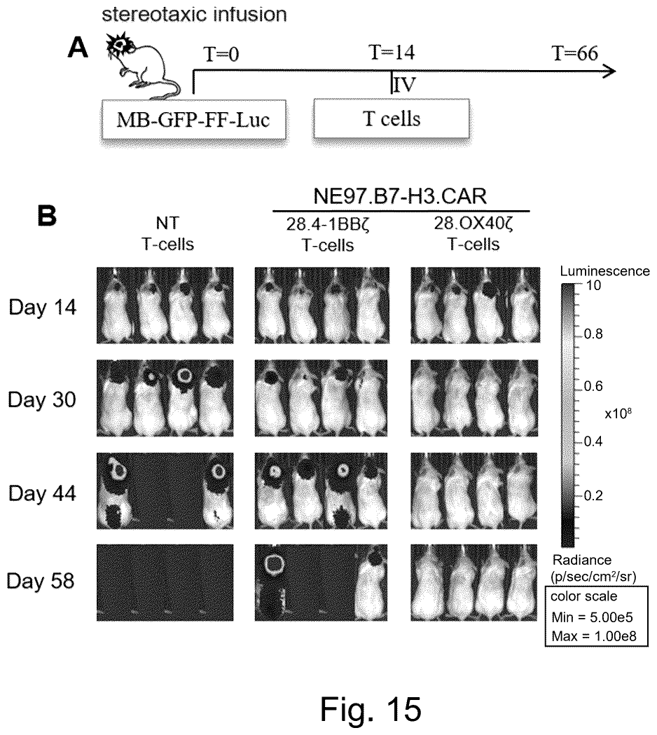

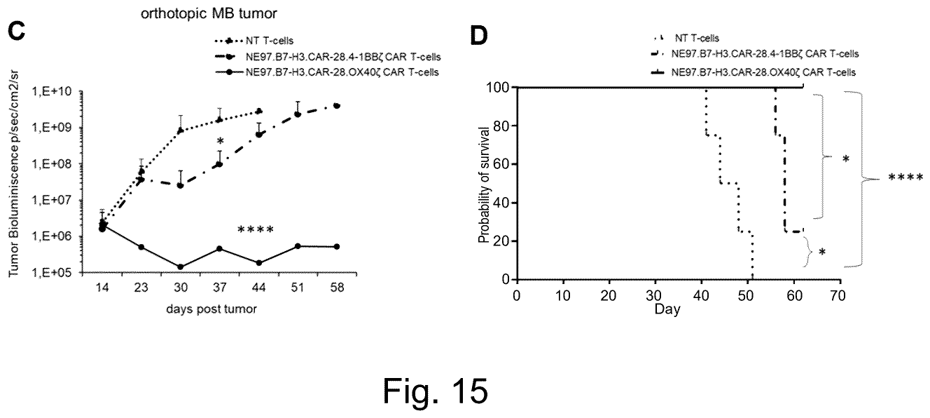

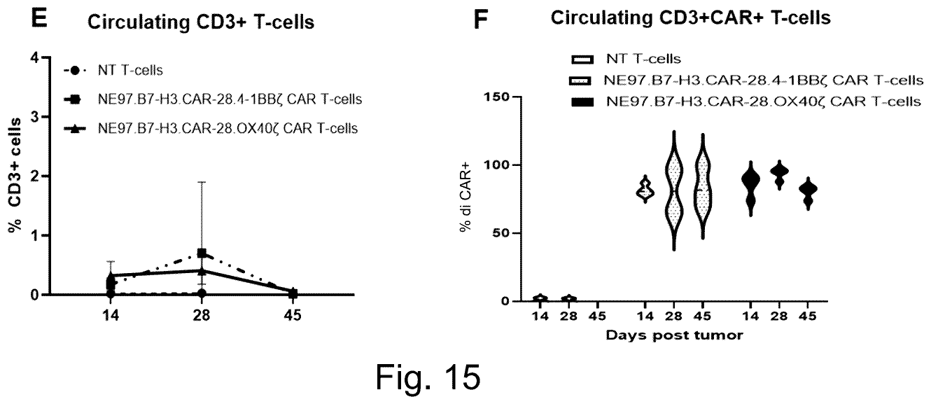

On the basis of the data reported in literature for CARs design in the contest of solid tumours models, CAR T-cells containing CD28.4-1BB outperforms both third generation CAR T-cells with CD28.OX40 costimulatory domain and second generation CARs containing CD28, OX40 or 4-1BB domains in neuroblastoma models (see page 5 of Quintarelli and al. report (PMID: 29872565). Moreover, Andreas A Hombach and al. reported that CD28 outperforms respect to the combined CD28-OX40 “super-stimulation” in cytokine-induced killer cells (CIK) armed with chimeric antigen receptors (PMID: 23985696). Therefore, according the above-mentioned results, a person skilled in the art would not be tempted to include CD28.OX40 (as a costimulatory domain) in the third generation of CAR to treat solid tumors. Surprisingly, the experimental data described below in in vivo solid tumor models clearly show the superiority of NE97.B7-H3.CAR T-cells with the CD28-OX40 costimulation domain, in terms of production of tumor-induced activating cytokines (FIG. 12), cytotoxic activity (FIG. 14 and FIG. 15), transduction efficiency of CAR T cells in vitro (FIG. 2B) and persistence in long term in vivo model (FIG. 15F).

Importantly, as mentioned above, all known B7-H3.CAR are characterized by the lack of a safety switch. However, the B7-H3 protein, despite being expressed at very low levels in healthy tissues, was weakly expressed by the cells of the lung, prostate, uterus and adrenal gland (PMID: 32346608). Therefore, the introduction of an inducible suicide gene (safety switch) into CAR is highly desirable because it makes the therapeutic proposal clinically safer. However, it is not so simple even for a skilled person to insert an inducible suicide gene into the final construct of CAR without reducing the transduction efficiency, the proliferation kinetics or the lytic capacity of gene modified effector cells against the tumor cells.

Surprisingly, the CARs according to the present invention comprise a functional safety switch (FIG. 3C), without altering the safety profile of CAR T cells (FIG. 3) or killing activity.

As mentioned above, the trackable markers that are mostly used in known B7-H3.CARs are: GFP, mCherry and tdTomato. These trackable markers are intracellular reporter molecules to evaluate gene transfer and expression, which can be detected in living cells without selection or staining. However, preclinical studies in animal in vivo models have shown that the GFP protein can induce the expansion of anti GFP T lymphocytes. This finding advices against the use of the B7-H3.CARs comprising the above mentioned trackable markers in the clinic setting (PMID: 10455440)(PMID: 27435468).

B7-H3.CARs constructs according to the present invention do not present this disadvantage since they comprise a ΔCD34 flag, represented by the extracellular domain fragment of human CD34, instead of GFP, mCherry and tdTomato.

All these results make it highly plausible that the constructs according to the present invention can be used to treat efficiently either B7-H3+leukemias/lymphomas or solid B7H3+tumour-bearing-patients.

In addition, in recent years, researchers reveal that B7-H3 is involved in the pathogenesis of various autoimmune diseases, such as systemic lupus erythematosus (SLE), rheumatoid arthritis (RA), multiple sclerosis (MS), Sjögren's syndrome (SS), ankylosing spondylitis (AS). The noticeable hallmarks of autoimmune diseases are the breakdown of self-tolerance and the self-attack of immune system, that is, our own immune system fails to distinguish self from non-self. (PMID: 26212387). Therefore, on the basis of the experimental results described below, it is plausible for a person skilled in the art that the anti B7-H3 CAR, the nucleotide sequence, the vector, the cell and the pharmaceutical composition according to the present invention is affective in the treatment of autoimmune diseases.

Therefore, it is a specific object of the present invention, anti-B7-H3 chimeric antigen receptor comprising or consisting of, from the N-terminus to the C-terminus:
a) a signal peptide,
b) an anti B7-H3 single chain antibody domain,
c) a hinge,
d) a trans membrane domain,
e) a co-stimulatory signaling domain, and
f) CD3Zeta chain sequence,
wherein said anti B7-H3 single chain antibody domain comprises or consists of anti B7-H3 NE97 hybridoma VL sequence and anti B7-H3 NE97 hybridoma VH sequence linked each other by a linker, and wherein
anti B7-H3 NE97 hybridoma VL sequence comprises CDR1 sequence EIIYSY (SEQ ID NO:1), CDR2 sequence NAK and CDR3 sequence QHHYGTPPYT (SEQ ID NO:2), whereas
anti B7-H3 NE97 hybridoma VH sequence comprises CDR1 sequence GFTFSSYG (SEQ ID NO:3), CDR2 sequence INSGGSYI (SEQ ID NO:4) and CDR3 sequence ARHEGLPLDY (SEQ ID NO:5).

According to the present invention,

anti B7-H3 NE97 hybridoma VL sequence can
comprise or consist of
(SEQ ID NO: 6)
DIQMTQSPASLSASVGETVTITCRASEIIYSYLAWYQQKQGKSPQ
LLVYNAKTLVEGVPSRFSGSGSGTQFSLKINSLQPEDFGSYYCQH
HYGTPPYTFGGGTKLEIK,
and
anti B7-H3 NE97 hybridoma VH sequence can
comprise or consist of
(SEQ ID NO: 7)
EVQLVESGGDLVKPGGSLKLSCAASGFTFSSYGMSWVRQTPDKRL
EWVATINSGGSYIYYPDSVKGRFTISRDNAENTLYLQMSSLKSED
TAMYYCARHEGLPLDYWGQGTTLTVSS.

Sequence SEQ ID NO:6 comprises CDR1 in position 27-32, CDR2 in position 50-52 and CDR3 in position 89-98. Sequence SEQ ID NO:7 comprises CDR1 in position 26-33, CDR2 in position 51-58 and CDR3 in position 97-10.

According to present invention, Anti-B7-H3 chimeric antigen receptor according to anyone of claims 1-2, wherein the linker which links anti B7-H3 NE97 hybridoma VL sequence and anti B7-H3 NE97 hybridoma VH sequence can be a short flexible linker glycines-rich with a length from 7 to 14 amino acids, such as from 7 to 12, from 7 to 10 or 8 amino acids, such as for example (G4S)2 linker GGGGSGGGG (SEQ ID NO:8), G4SG2 linker GGGGSGG (SEQ ID NO:9) or G3SG4 linker GGGSGGGG (SEQ ID NO:10) SG4SG3 linker SGGGGSGGG (SEQ ID NO:54), (SG4)2 S linker SGGGGSGGGGS (SEQ ID NO:55), (SG4)2 SG linker SGGGGSGGGGSG (SEQ ID NO:56), (SG4)2 SG3 linker SGGGGSGGGGSGGG linker (SEQ ID NO:57), (SG4)2 SGGGGSGGGG (SEQ ID NO:58), (SG4)2 SG2 SGGGGSGGGGSGG (SEQ ID NO:59), preferably, G3SG4 linker.

As mentioned above, a linker is useful in order to prevent epitope masking in CAR+ tumor blasts, said CAR T cells being able to decrease the potential risk of tumor relapse, for example in cell B leukemia. In addition, CAR T cells according to the present invention provides increased safety also in the treatment of autoimmune diseases caused by B cells producing auto-antibodies.

According to the present invention, the hinge of the anti-B7-H3 chimeric antigen receptor can comprise or consist of one or more of the following hinges:

hinge Spacer-CD8α
(SEQ ID NO: 11)
PAPRPPTPAPTIASQPLSLRPEACRPAAGGAVHTRGLDFA
(nucleotide ID NO: M12828.1 and Protein
ID NO: AAB04637.1);
CD8stalk:
(SEQ ID NO: 12)
TTTPAPRPPTPAPTIASQPLSLRPEACRPAAGGAVHTRGLDFACD;
Hinge CD28
(SEQ ID NO: 13)
EVMYPPPYLDNEKSNGTIIHVKGKHLCPSPLFPGPSKP;
hinge CH2-CH3
(SEQ ID NO: 14)
ESKYGPPCPSCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLGK;
or
hinge CH3:
(SEQ ID NO: 15)
ESKYGPPCPSCPGQPREPQVYTLPPSQEEMTKNQVSLTCLVKGFY
PSDIAVEWESNGQPENNYKTTPPVLDSDGSFFLYSRLTVDKSRWQ
EGNVFSCSVMHEALHNHYTQKSLSLSLGK,
preferably hinge Spacer-CD8α
(SEQ ID NO: 11)
PAPRPPTPAPTIASQPLSLRPEACRPAAGGAVHTRGLDFA
(nucleotide ID NO: M12828.1 and Protein
ID NO: AAB04637.1).

The hinge can be linked, at the N terminus, to a trackable marker, said trackable marker being linked, optionally by a second linker, to the anti B7-H3 single chain antibody domain.

According to the present invention, the trackable marker of anti-B7-H3 chimeric antigen receptor can be chosen from the group consisting of:

ΔCD34:
(SEQ ID NO: 16)
ELPTQGTFSNVSTNVS
(nucleotide ID NO AB238231.1 and
Protein ID NO: BAE46748.1);
ΔCD19:
(SEQ ID. NO: 17)
PEEPLVKVEEGDNAVLQCLKGTSDGPTQQLTWSRESPLKPFLKLS
LGLPGLGIHMRPLAIWLFIFNVSQQMGGFYLCQPGPPSEKAWQPG
WTVNVEGSGELFRWNVSDLGGLGCGLKNRSSEGPSSPSGKLMSPK
LYVWAKDRPEIWEGEPPCLPPRDSLNQSLSQDLTMAPGSTLWLSC
GVPPDSVSRGPLSWTHVHPKGPKSLLSLELKDDRPARDMWVMETG
LLLPRATAQDAGKYYCHRGNLTMSFHLEITARPVLWHWLLRTGGW
K
(nucleotide ID NO: M21097.1 and
Protein ID NO: AAA35533.1);
NGFR:
(SEQ ID NO: 18)
KEACPTGLYTHSGECCKACNLGEGVAQPCGANQTVCEPCLDSVTF
SDVVSATEPCKPCTECVGLQSMSAPCVEADDAVCRCAYGYYQDET
TGRCEACRVCEAGSGLVFSCQDKQNTVCEECPDGTYSDEANHVDP
CLPCTVCEDTERQLRECTRWADAECEEIPGRWITRSTPPEGSDST
APSTQEPEAPPEQDLIASTVAGVVTTVMGSSQPVVTRGTTDN
(nucleotide ID NO: AK313654.1 and
Protein ID NO: BAG36408.1);
preferably ΔCD34:
(SEQ ID NO: 16)
ELPTQGTFSNVSTNVS
(nucleotide ID NO AB238231.1 and
Protein ID NO: BAE46748.1).

According to the present invention, the trans membrane domain of anti-B7-H3 chimeric antigen receptor can be chosen from the group consisting of CD8aTM: CDIYIWAPLAGTCGVLLLSLVIT (SEQ ID NO:19), (nucleotide ID NO NM_001768.6 and Protein ID NO: NP_001759.3); CD28TM: FWVLVVVGGVLACYSLLVTVAFIIFWV (SEQ ID NO:20) (nucleotide ID NO: BC112085.1 and Protein ID NO: AA112086.1); preferably CD8aTM: CDIYIWAPLAGTCGVLLLSLVIT (SEQ ID NO:19), (nucleotide ID NO: NM_001768.6 and Protein ID NO: NP_001759.3).

According to the present invention, the co-stimulatory signaling domain of anti-B7-H3 chimeric antigen receptor can be chosen from the group consisting of

CD28 cytoplasmic sequence:
(SEQ ID NO: 21)
RSKRSRLLHSDYMNMTPRRPGPTRKHYQPYAPPRDFAAYRS
(nucleotide ID NO: AF222341.1 and
Protein ID NO: AAF33792.1),
CD137 (4-1BB) sequence:
(SEQ ID NO: 22)
KRGRKKLLYIFKQPFMRPVQTTQEEDGCSCRFPEEEEGGCEL
(nucleotide ID NO: U03397.1 and
Protein NO: AAA53133.1),
OX40 sequence:
(SEQ ID NO: 23)
RDQRLPPDAHKPPGGGSFRTPIQEEQADAHSTLAKI
(nucleotide ID NO: NM_003327.3 and
Protein NO: NP_003318.1),
a sequence obtained by linking
CD28 cytoplasmic sequence:
(SEQ ID NO: 21)
RSKRSRLLHSDYMNMTPRRPGPTRKHYQPYAPPRDFAAYRS
(nucleotide ID NO: AF222341.1 and
Protein ID NO: AAF33792.1) to
CD137 (4-1BB) sequence:
(SEQ ID NO: 22)
KRGRKKLLYIFKQPFMRPVQTTQEEDGCSCRFPEEEEGGCEL
(nucleotide ID NO: U03397.1 and
Protein NO: AAA53133.1), or
a sequence obtained by linking
CD28 cytoplasmic sequence:
(SEQ ID NO: 21)
RSKRSRLLHSDYMNMTPRRPGPTRKHYQPYAPPRDFAAYRS
(nucleotide ID NO: AF222341.1 and
Protein ID NO: AAF33792.1), to
OX40 sequence:
(SEQ ID NO: 23)
RDQRLPPDAHKPPGGGSFRTPIQEEQADAHSTLAKI
(nucleotide ID NO: NM_003327.3 and
Protein NO: NP_003318.1)

According to the present invention, a co-stimulatory signaling domain with OX40 sequence is preferable.

According to the present invention, CD3-Zeta chain of anti-B7-H3 chimeric antigen receptor can be

(SEQ ID NO: 24)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR*
(nucleotide ID NO: J04132.1 And
Protein ID: AAA60394.1).

According to the present invention, the anti-B7-H3 chimeric antigen receptor can further comprise cytoplasmic moiety of CD8cyt, CD8a cytoplasmic (CD8a cyto):
LYCNHRN (SEQ ID NO:25) (nucleotide ID NO: NM_001768.6 and Protein ID NO: NP_001759.3) between the trans membrane domain and the co-stimulatory signaling domain.

According to the present invention, the signal peptide of anti-B7-H3 chimeric antigen can comprise or consist of MEFGLSWLFLVAILKGVQC (SEQ ID NO:26) (nucleotide ID NO: AB776838.1 and Protein ID NO: BAN63131.1).

According to an embodiment of the present invention, the anti-B7-H3 chimeric antigen receptor comprises or consists of the following sequence:

(NE97.B7-H3.CAR-28.4-1BBζ T-cells)
(SEQ ID NO: 27)
MEFGLSWLFLVAILKGVQCSRDIQMTQSPASLSASVGETVTITCR
ASEIIYSYLAWYQQKQGKSPQLLVYNAKTLVEGVPSRFSGSGSGT
QFSLKINSLQPEDFGSYYCQHHYGTPPYTFGGGTKLEIKGGGSGG
GGEVQLVESGGDLVKPGGSLKLSCAASGFTFSSYGMSWVRQTPDK
RLEWVATINSGGSYIYYPDSVKGRFTISRDNAENTLYLQMSSLKS
EDTAMYYCARHEGLPLDYWGQGTTLTVSSACELPTQGTFSNVSTN
VSPAPRPPTPAPTIASQPLSLRPEACRPAAGGAVHTRGLDFACDI
YIWAPLAGTCGVLLLSLVITLYCNHRNEFRSKRSRLLHSDYMNMT
PRRPGPTRKHYQPYAPPRDFAAYRSKRGRKKLLYIFKQPFMRPVQ
TTQEEDGCSCRFPEEEEGGCELRVKFSRSADAPAYQQGQNQLYNE
LNLGRREEYDVLDKRRGRDPEMGGKPRRKNPQEGLYNELQKDKMA
EAYSEIGMKGERRRGKGHDGLYQGLSTATKDTYDALHMQALPPR*
Or
(NE97.B7-H3.CAR-28.OX40ζ T-cells)
(SEQ ID NO: 28)
MEFGLSWLFLVAILKGVQCSRDIQMTQSPASLSASVGETVTITCR
ASEIIYSYLAWYQQKQGKSPQLLVYNAKTLVEGVPSRFSGSGSGT
QFSLKINSLQPEDFGSYYCQHHYGTPPYTFGGGTKLEIKGGGSGG
GGEVQLVESGGDLVKPGGSLKLSCAASGFTFSSYGMSWVRQTPDK
RLEWVATINSGGSYIYYPDSVKGRFTISRDNAENTLYLQMSSLKS
EDTAMYYCARHEGLPLDYWGQGTTLTVSSACELPTQGTFSNVSTN
VSPAPRPPTPAPTIASQPLSLRPEACRPAAGGAVHTRGLDFACDI
YIWAPLAGTCGVLLLSLVITLYCNHRNEFRSKRSRLLHSDYMNMT
PRRPGPTRKHYQPYAPPRDFAAYRSRDQRLPPDAHKPPGGGSFRT
PIQEEQADAHSTLAKIRVKFSRSADAPAYQQGQNQLYNELNLGRR
EEYDVLDKRRGRDPEMGGKPRRKNPQEGLYNELQKDKMAEAYSEI
GMKGERRRGKGHDGLYQGLSTATKDTYDALHMQALPPR*.

More in detail, (NE97)B7-H3.CAR-ΔCD34.CD8a.CD28.4-1BBζ comprises:

A Signal peptide
(SEQ ID NO: 26)
MEFGLSWLFLVAILKGVQC
(nucleotide ID NO: AB776838.1 and Protein
ID NO: BAN63131.1), which is linked by
the Linker (connection sequence) SR to
NE97 VL
(SEQ ID NO: 6)
DIQMTQSPASLSASVGETVTITCRASEIIYSYLAWYQQKQGKSPQ
LLVYNAKTLVEGVPSRFSGSGSGTQFSLKINSLQPEDFGSYYCQH
HYGTPPYTFGGGTKLEIK
Flex linker
(SEQ ID NO: 10)
GGGSGGGG.
NE97 VH
(SEQ ID NO: 7)
EVQLVESGGDLVKPGGSLKLSCAASGFTFSSYGMSWVRQTPDKRL
EWVATINSGGSYIYYPDSVKGRFTISRDNAENTLYLQMSSLKSED
TAMYYCARHEGLPLDYWGQGTTLTVSS
Link (connection sequence)
AC
ΔCD34
(SEQ ID NO: 16)
ELPTQGTFSNVSTNVS
(nucleotide ID NO AB238231.1
and Protein ID NO: BAE46748.1);
Hinge Spacer-CD8α
(SEQ ID NO: 11)
PAPRPPTPAPTIASQPLSLRPEACRPAAGGAVHTRGLDFA
(nucleotide ID NO: M12828.1 and
Protein ID NO: AAB04637.1);
CD8a TM
(SEQ ID NO: 19)
CDIYIWAPLAGTCGVLLLSLVIT,
(nucleotide ID NO: NM_001768.6 and
Protein ID NO: NP_001759.3);
CD8a cytoplasmic (CD8a cyto):
(SEQ ID NO: 25)
LYCNHRN
(nucleotide ID NO: NM_001768.6 and Protein ID
NO: NP_001759.3)
Linked of connection
EF
CD28 cytoplasmic sequence:
(SEQ ID NO: 21)
RSKRSRLLHSDYMNMTPRRPGPTRKHYQPYAPPRDFAAYRS
(nucleotide ID NO: AF222341.1
and Protein ID NO: AAF33792.1),
(4-1BB) sequence:
(SEQ ID NO: 22)
KRGRKKLLYIFKQPFMRPVQTTQEEDGCSCRFPEEEEGGCEL
(nucleotide ID NO: U03397.1 and Protein NO:
AAA53133.1),
Linked to CD3-Zeta chain:
(SEQ ID NO: 24)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR*
(nucleotide ID NO: J04132.1 And Protein ID:
AAA60394.1).
(NE97)B7-H3.CAR-ΔCD34.CD8a.CD28.OX407 comprises:
A Signal peptide
(SEQ ID NO: 26)
MEFGLSWLFLVAILKGVQC
(nucleotide ID NO: AB776838.1
and Protein ID NO: BAN63131.1), which
is linked by the Linker (connection
sequence) SR to
NE97 VL
(SEQ ID NO: 6)
DIQMTQSPASLSASVGETVTITCRASEIIYSYLAWYQQKQGKSPQ
LLVYNAKTLVEGVPSRFSGSGSGTQFSLKINSLQPEDFGSYYCQH
HYGTPPYTFGGGTKLEIK
Flex
(SEQ ID NO: 10)
GGGSGGGG.
NE97 VH
(SEQ ID NO: 7)
EVQLVESGGDLVKPGGSLKLSCAASGFTFSSYGMSWVRQTPDKRL
EWVATINSGGSYIYYPDSVKGRFTISRDNAENTLYLQMSSLKSED
TAMYYCARHEGLPLDYWGQGTTLTVSS
Link (connection sequence)
AC
ΔCD34
(SEQ ID NO: 16)
ELPTQGTFSNVSTNVS
(nucleotide ID NO AB238231.1 and
Protein ID NO: BAE46748.1);
Hinge Spacer-CD8α
(SEQ ID NO: 11)
PAPRPPTPAPTIASQPLSLRPEACRPAAGGAVHTRGLDFA
(nucleotide ID NO: M12828.1 and Protein
ID NO: AAB04637.1);
CD8a TM
(SEQ ID NO: 19)
CDIYIWAPLAGTCGVLLLSLVIT,
(nucleotide ID NO:
NM_001768.6 and Protein ID NO: NP_001759.3);
CD8a cytoplasmic (CD8a cyto)
(SEQ ID NO: 25)
LYCNHRN
(nucleotide ID NO: NM_001768.6 and Protein ID
NO: NP_001759.3)
Linked of connection
EF
CD28 cytoplasmic sequence
(SEQ ID NO: 21)
RSKRSRLLHSDYMNMTPRRPGPTRKHYQPYAPPRDFAAYRS
(nucleotide ID NO: AF222341.1 and Protein
ID NO: AAF33792.1),
OX40 sequence
(SEQ ID NO: 23)
RDQRLPPDAHKPPGGGSFRTPIQEEQADAHSTLAKI
(nucleotide ID NO: NM_003327.3 and Protein
NO: NP_003318.1)
and
CD3-Zeta chain:
(SEQ ID NO: 24)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR*
(nucleotide ID NO: J04132.1 And Protein ID:
AAA60394.1).

The present invention concerns also a nucleotide sequence comprising or consisting of a nucleotide sequence which encodes an anti-B7-H3 chimeric antigen receptor according to the above.

According to an embodiment of the present invention, the nucleotide sequence is a nucleotide sequence wherein

anti B7-H3 NE97 hybridoma VL sequence
is encoded by the nucleotide
sequence
(SEQ ID NO: 29)
GACATCCAGATGACTCAGTCTCCAGCCTCCCTATCTGCATCTGTG
GGAGAAACTGTCACCATCACATGTCGAGCAAGTGAGATTATTTAC
AGTTATTTAGCATGGTATCAGCAGAAACAGGGAAAATCTCCTCAG
CTCCTGGTCTATAATGCAAAAACCTTAGTAGAAGGTGTGCCATCA
AGGTTCAGTGGCAGTGGATCAGGCACACAGTTTTCTCTGAAGATC
AACAGCCTGCAGCCTGAAGATTTTGGGAGTTATTACTGTCAACAT
CATTATGGTACTCCTCCATACACGTTCGGAGGGGGGACCAAGCTG
GAAATAAAA,
and
anti B7-H3 NE97 hybridoma VH sequence
is encoded by the nucleotide
sequence
(SEQ ID NO: 30)
GAGGTGCAGCTGGTGGAGTCTGGGGGAGACTTAGTGAAGCCTGGA
GGGTCCCTGAAACTCTCCTGTGCAGCCTCTGGATTCACTTTCAGT
AGCTATGGCATGTCTTGGGTTCGCCAGACTCCAGACAAGAGGCTG
GAGTGGGTCGCAACCATTAATAGTGGTGGTAGTTACATCTACTAT
CCAGACAGTGTGAAGGGGCGATTCACCATCTCCAGAGACAATGCC
GAGAACACCCTGTACCTGCAAATGAGCAGTCTGAAGTCTGAAGAC
ACAGCCATGTATTACTGTGCAAGACATGAAGGGTTACCCCTTGAC
TACTGGGGCCAAGGCACCACTCTCACAGTCTCCTCA.
In sequence SEQ ID NO: 29 the sequences
encoding CDR1, CDR2 and
CDR3 are the following:
CDR1:
(SEQ ID NO: 31)
GAGATTATTTACAGTTAT
in position 79-96;
CDR2:
AATGCAAAA
in position 148-156:
CDR3:
(SEQ ID NO: 32)
CAACATCATTATGGTACTCCTCCATACACG
in position 265-287
In sequence SEQ ID NO: 30 the sequences
encoding CDR1, CDR2 and
CDR3 are the following:
CDR1:
(SEQ ID NO: 33)
GGATTCACTTTCAGTAGCTATGGC
in position 76-99;
CDR2:
(SEQ ID NO: 34)
ATTAATAGTGGTGGTAGTTACATC
in position 151-174;
CDR3:
(SEQ ID NO: 35)
GCAAGACATGAAGGGTTACCCCTTGACTAC
in position 289-294.

According to an embodiment of the present invention, the nucleotide sequence encoding anti-B7-H3 chimeric antigen receptor is:

(NE97)B7-H3.CAR-ΔCD34.CD8a.CD28.4-1BBζ
(SEQ ID NO: 36)
ATGGAGTTTGGGCTCTCCTGGCTCTTCCTGGTCGCGATTCTGAAGGGGGTCC
AGTGTTCACGAGACATCCAGATGACTCAGTCTCCAGCCTCCCTATCTGCATCT
GTGGGAGAAACTGTCACCATCACATGTCGAGCAAGTGAGATTATTTACAGTTA
TTTAGCATGGTATCAGCAGAAACAGGGAAAATCTCCTCAGCTCCTGGTCTATA
ATGCAAAAACCTTAGTAGAAGGTGTGCCATCAAGGTTCAGTGGCAGTGGATC
AGGCACACAGTTTTCTCTGAAGATCAACAGCCTGCAGCCTGAAGATTTTGGG
AGTTATTACTGTCAACATCATTATGGTACTCCTCCATACACGTTCGGAGGGGG
GACCAAGCTGGAAATAAAAGGCGGAGGTTCAGGCGGAGGAGGGGAGGTGC
AGCTGGTGGAGTCTGGGGGAGACTTAGTGAAGCCTGGAGGGTCCCTGAAAC
TCTCCTGTGCAGCCTCTGGATTCACTTTCAGTAGCTATGGCATGTCTTGGGTT
CGCCAGACTCCAGACAAGAGGCTGGAGTGGGTCGCAACCATTAATAGTGGT
GGTAGTTACATCTACTATCCAGACAGTGTGAAGGGGCGATTCACCATCTCCA
GAGACAATGCCGAGAACACCCTGTACCTGCAAATGAGCAGTCTGAAGTCTGA
AGACACAGCCATGTATTACTGTGCAAGACATGAAGGGTTACCCCTTGACTACT
GGGGCCAAGGCACCACTCTCACAGTCTCCTCAGCATGCGAACTTCCTACTCA
GGGGACTTTCTCAAACGTTAGCACAAACGTAAGTCCCGCCCCAAGACCCCCC
ACACCTGCGCCGACCATTGCTTCTCAACCCCTGAGTTTGAGACCCGAGGCCT
GCCGGCCAGCTGCCGGCGGGGCCGTGCATACAAGAGGACTCGATTTCGCTT
GCGACATCTACATCTGGGCTCCCCTCGCTGGCACCTGTGGGGTGCTGCTGC
TGTCACTCGTGATCACCCTTTATTGCAACCATCGAAACGAATTCAGAAGTAAA
CGGTCAAGGCTTCTGCACAGCGATTATATGAATATGACACCAAGAAGACCTG
GTCCAACCCGGAAACACTATCAGCCCTACGCGCCCCCTAGAGACTTCGCAGC
ATACCGCTCTAAGAGAGGGAGAAAAAAATTGCTCTATATTTTTAAACAACCATT
TATGAGGCCCGTACAGACAACTCAGGAAGAGGATGGCTGTAGTTGCCGCTTC
CCAGAGGAGGAGGAAGGAGGCTGCGAGTTGAGAGTTAAATTCAGTAGAAGT
GCGGATGCGCCTGCTTACCAGCAGGGCCAGAACCAACTGTACAATGAACTGA
ATCTCGGGCGCCGAGAAGAGTATGACGTCCTCGATAAGCGGAGGGGTAGGG
ATCCTGAAATGGGGGGAAGCCAAGAAGAAAAAACCCCCAGGAAGGACTGTA
TAACGAACTTCAGAAGGACAAGATGGCAGAGGCCTACTCTGAGATTGGCATG
AAAGGCGAACGACGGCGCGGTAAAGGTCATGACGGGCTGTACCAGGGCCTG
TCCACAGCGACGAAGGACACTTACGACGCCCTGCACATGCAGGCACTCCCC
CCCAGGTGA
or
NE97-B7-H3.CAR-ΔCD34.CD8a.CD28.OX40ζ
(SEQ ID NO: 37)
ATGGAGTTTGGGCTCTCCTGGCTCTTCCTGGTCGCGATTCTGAAGGGGGTCC
AGTGTTCACGAGACATCCAGATGACTCAGTCTCCAGCCTCCCTATCTGCATCT
GTGGGAGAAACTGTCACCATCACATGTCGAGCAAGTGAGATTATTTACAGTTA
TTTAGCATGGTATCAGCAGAAACAGGGAAAATCTCCTCAGCTCCTGGTCTATA
ATGCAAAAACCTTAGTAGAAGGTGTGCCATCAAGGTTCAGTGGCAGTGGATC
AGGCACACAGTTTTCTCTGAAGATCAACAGCCTGCAGCCTGAAGATTTTGGG
AGTTATTACTGTCAACATCATTATGGTACTCCTCCATACACGTTCGGAGGGGG
GACCAAGCTGGAAATAAAAGGCGGAGGTTCAGGCGGAGGAGGGGAGGTGC
AGCTGGTGGAGTCTGGGGGAGACTTAGTGAAGCCTGGAGGGTCCCTGAAAC
TCTCCTGTGCAGCCTCTGGATTCACTTTCAGTAGCTATGGCATGTCTTGGGTT
CGCCAGACTCCAGACAAGAGGCTGGAGTGGGTCGCAACCATTAATAGTGGT
GGTAGTTACATCTACTATCCAGACAGTGTGAAGGGGCGATTCACCATCTCCA
GAGACAATGCCGAGAACACCCTGTACCTGCAAATGAGCAGTCTGAAGTCTGA
AGACACAGCCATGTATTACTGTGCAAGACATGAAGGGTTACCCCTTGACTACT
GGGGCCAAGGCACCACTCTCACAGTCTCCTCAGCATGCGAACTTCCTACTCA
GGGGACTTTCTCAAACGTTAGCACAAACGTAAGTCCCGCCCCAAGACCCCCC
ACACCTGCGCCGACCATTGCTTCTCAACCCCTGAGTTTGAGACCCGAGGCCT
GCCGGCCAGCTGCCGGCGGGGCCGTGCATACAAGAGGACTCGATTTCGCTT
GCGACATCTACATCTGGGCTCCCCTCGCTGGCACCTGTGGGGTGCTGCTGC
TGTCACTCGTGATCACCCTTTATTGCAACCATCGAAACGAATTCAGAAGTAAA
CGGTCAAGGCTTCTGCACAGCGATTATATGAATATGACACCAAGAAGACCTG
GTCCAACCCGGAAACACTATCAGCCCTACGCGCCCCCTAGAGACTTCGCAGC
ATACCGCTCTCGCGATCAAAGACTCCCGCCCGATGCCCACAAACCCCCTGGC
GGGGGCAGCTTTAGGACACCCATTCAAGAAGAGCAGGCAGACGCCCACAGC
ACCTTGGCCAAAATTAGAGTTAAATTCAGTAGAAGTGCGGATGCGCCTGCTTA
CCAGCAGGGCCAGAACCAACTGTACAATGAACTGAATCTCGGGCGCCGAGA
AGAGTATGACGTCCTCGATAAGCGGAGGGGTAGGGATCCTGAAATGGGTGG
GAAGCCAAGAAGAAAAAACCCCCAGGAAGGACTGTATAACGAACTTCAGAAG
GACAAGATGGCAGAGGCCTACTCTGAGATTGGCATGAAAGGCGAACGACGG
CGCGGTAAAGGTCATGACGGGCTGTACCAGGGCCTGTCCACAGCGACGAAG
GACACTTACGACGCCCTGCACATGCAGGCACTCCCCCCCAGGTGA.

According to the present invention, the nucleotide sequence can further comprise a nucleotide sequence encoding a suicide gene inducible amino acid sequence linked to the nucleotide sequence encoding said chimeric antigen receptor by a nucleotide sequence encoding a 2A self-cleaving peptide.

According to an embodiment of the present invention, the suicide gene inducible amino acid sequence can be a chimeric Caspase-9 polypeptide or can comprise a herpes simplex virus thymidine kinase.

Therefore, in the cell, the polynucleotide 2A self-cleaving peptide cuts the peptide comprising the suicide gene inducible amino acid sequence and the chimeric antigen receptor in two separate peptides, i.e., the suicide gene inducible and the chimeric antigen receptor amino acid sequences.

According to an embodiment of the present invention, the nucleotide sequence is

```
SFG.iC9-2A-(NE97)B7-H3.CAR-ΔCD34.CD8a.CD28.4-1BBζ
  (SEQ ID NO: 38)
ATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTTTAT
GCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGT
GCCACTGGGCACCCAGGCATCATCCCACCACATGCCACTCTCGTCTTCGATG
TGGAGCTTCTAAAACTGGAATCTGGCGGTGGATCCGGAGTCGACGGATTTGG
TGATGTCGGTGCTCTTGAGAGTTTGAGGGGAAATGCAGATTTGGCTTACATC
CTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGA
AGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGG
ACCACGGTGCTCTGGACTGCTGCGTGGTGGTCATTCTCTCTCACGGCTGTCA
GGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGC
CTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTG
GCAGTAACCCCGAGCCAGATGCCACCCCGTTCCAGGAAGGTTTGAGGACCT
TCGACCAGCTGGACGCCATATCTAGTTTGCCCACACCCAGTGACATCTTTGT
GTCCTACTCTACTTTCCCAGGTTTTGTTTCCTGGAGGGACCCCAAGAGTGGC
TCCTGGTACGTTGAGACCCTGGACGACATCTTTGAGCAGTGGGCTCACTCTG
AAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTT
TAAAACATCAGCTAGCAGAGCCGAGGGCAGGGGAAGTCTTCTAACATGCGG
GGACGTGGAGGAAAATCCCGGGCCCATGGAGTTTGGGCTCTCCTGGCTCTT
CCTGGTCGCGATTCTGAAGGGGGTCCAGTGTTCACGAGACATCCAGATGACT
CAGTCTCCAGCCTCCCTATCTGCATCTGTGGGAGAAACTGTCACCATCACAT
GTCGAGCAAGTGAGATTATTTACAGTTATTTAGCATGGTATCAGCAGAAACAG
GGAAAATCTCCTCAGCTCCTGGTCTATAATGCAAAAACCTTAGTAGAAGGTGT
GCCATCAAGGTTCAGTGGCAGTGGATCAGGCACACAGTTTTCTCTGAAGATC
AACAGCCTGCAGCCTGAAGATTTTGGGAGTTATTACTGTCAACATCATTATGG
TACTCCTCCATACACGTTCGGAGGGGGGACCAAGCTGGAAATAAAAGGCGG
AGGTTCAGGCGGAGGAGGGGAGGTGCAGCTGGTGGAGTCTGGGGGAGACT
TAGTGAAGCCTGGAGGGTCCCTGAAACTCTCCTGTGCAGCCTCTGGATTCAC
TTTCAGTAGCTATGGCATGTCTTGGGTTCGCCAGACTCCAGACAAGAGGCTG
GAGTGGGTCGCAACCATTAATAGTGGTGGTAGTTACATCTACTATCCAGACA
GTGTGAAGGGGCGATTCACCATCTCCAGAGACAATGCCGAGAACACCCTGTA
CCTGCAAATGAGCAGTCTGAAGTCTGAAGACACAGCCATGTATTACTGTGCA
AGACATGAAGGGTTACCCCTTGACTACTGGGGCCAAGGCACCACTCTCACAG
TCTCCTCAGCATGCGAACTTCCTACTCAGGGGACTTTCTCAAACGTTAGCACA
AACGTAAGTCCCGCCCCAAGACCCCCCACACCTGCGCCGACCATTGCTTCTC
AACCCCTGAGTTTGAGACCCGAGGCCTGCCGGCCAGCTGCCGGCGGGGCC
GTGCATACAAGAGGACTCGATTTCGCTTGCGACATCTACATCTGGGCTCCCC
TCGCTGGCACCTGTGGGGTGCTGCTGCTGTCACTCGTGATCACCCTTTATTG
CAACCATCGAAACGAATTCAGAAGTAAACGGTCAAGGCTTCTGCACAGCGAT
TATATGAATATGACACCAAGAAGACCTGGTCCAACCCGGAAACACTATCAGC
CCTACGCGCCCCCTAGAGACTTCGCAGCATACCGCTCTAAGAGAGGGAGAA
AAAAATTGCTCTATATTTTTAAACAACCATTTATGAGGCCCGTACAGACAACTC
AGGAAGAGGATGGCTGTAGTTGCCGCTTCCCAGAGGAGGAGGAAGGAGGCT
GCGAGTTGAGAGTTAAATTCAGTAGAAGTGOGGATGCGCCTGCTTACCAGCA
GGGCCAGAACCAACTGTACAATGAACTGAATCTCGGGCGCCGAGAAGAGTAT
GACGTCCTCGATAAGCGGAGGGGTAGGGATCCTGAAATGGGTGGGAAGCCA
AGAAGAAAAAACCCCCAGGAAGGACTGTATAACGAACTTCAGAAGGACAAGA
TGGCAGAGGCCTACTCTGAGATTGGCATGAAAGGCGAACGACGGCGCGGTA
AAGGTCATGACGGGCTGTACCAGGGCCTGTCCACAGCGACGAAGGACACTT
ACGACGCCCTGCACATGCAGGCACTCCCCCCCAGGTGA
or
SFG.iC9-2A-(NE97)B7-H3.CAR-ΔCD34.CD8a.CD28.OX40ζ
  (SEQ ID NO: 39)
ATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTTTAT
GCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGT
GCCACTGGGCACCCAGGCATCATCCCACCACATGCCACTCTCGTCTTCGATG
TGGAGCTTCTAAAACTGGAATCTGGCGGTGGATCCGGAGTCGACGGATTTGG
TGATGTCGGTGCTCTTGAGAGTTTGAGGGGAAATGCAGATTTGGCTTACATC
CTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGA
AGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGG
ACCACGGTGCTCTGGACTGCTGCGTGGTGGTCATTCTCTCTCACGGCTGTCA
GGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGC
CTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTG
GCAGTAACCCCGAGCCAGATGCCACCCCGTTCCAGGAAGGTTTGAGGACCT
TCGACCAGCTGGACGCCATATCTAGTTTGCCCACACCCAGTGACATCTTTGT
GTCCTACTCTACTTTCCCAGGTTTTGTTTCCTGGAGGGACCCCAAGAGTGGC
TCCTGGTACGTTGAGACCCTGGACGACATCTTTGAGCAGTGGGCTCACTCTG
AAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTT
TAAAACATCAGCTAGCAGAGCCGAGGGCAGGGGAAGTCTTCTAACATGCGG
GGACGTGGAGGAAAATCCCGGGCCCATGGAGTTTGGGCTCTCCTGGCTCTT
CCTGGTCGCGATTCTGAAGGGGGTCCAGTGTTCACGAGACATCCAGATGACT
CAGTCTCCAGCCTCCCTATCTGCATCTGTGGGAGAAACTGTCACCATCACAT
GTCGAGCAAGTGAGATTATTTACAGTTATTTAGCATGGTATCAGCAGAAACAG
GGAAAATCTCCTCAGCTCCTGGTCTATAATGCAAAAACCTTAGTAGAAGGTGT
GCCATCAAGGTTCAGTGGCAGTGGATCAGGCACACAGTTTTCTCTGAAGATC
AACAGCCTGCAGCCTGAAGATTTTGGGAGTTATTACTGTCAACATCATTATGG
TACTCCTCCATACACGTTCGGAGGGGGGACCAAGCTGGAAATAAAAGGCGG
AGGTTCAGGCGGAGGAGGGGAGGTGCAGCTGGTGGAGTCTGGGGGAGACT
TAGTGAAGCCTGGAGGGTCCCTGAAACTCTCCTGTGCAGCCTCTGGATTCAC
TTTCAGTAGCTATGGCATGTCTTGGGTTCGCCAGACTCCAGACAAGAGGCTG
GAGTGGGTCGCAACCATTAATAGTGGTGGTAGTTACATCTACTATCCAGACA
GTGTGAAGGGGCGATTCACCATCTCCAGAGACAATGCCGAGAACACCCTGTA
CCTGCAAATGAGCAGTCTGAAGTCTGAAGACACAGCCATGTATTACTGTGCA
AGACATGAAGGGTTACCCCTTGACTACTGGGGCCAAGGCACCACTCTCACAG
TCTCCTCAGCATGCGAACTTCCTACTCAGGGGACTTTCTCAAACGTTAGCACA
AACGTAAGTCCCGCCCCAAGACCCCCCACACCTGCGCCGACCATTGCTTCTC
AACCCCTGAGTTTGAGACCCGAGGCCTGCCGGCCAGCTGCCGGGGGGGCC
GTGCATACAAGAGGACTCGATTTCGCTTGCGACATCTACATCTGGGCTCCCC
TCGCTGGCACCTGTGGGGTGCTGCTGCTGTCACTCGTGATCACCCTTTATTG
CAACCATCGAAACGAATTCAGAAGTAAACGGTCAAGGCTTCTGCACAGCGAT
TATATGAATATGACACCAAGAAGACCTGGTCCAACCCGGAAACACTATCAGC
CCTACGCGCCCCCTAGAGACTTCGCAGCATACCGCTCTCGCGATCAAAGACT
CCCGCCCGATGCCCACAAACCCCCTGGCGGGGGCAGCTTTAGGACACCCAT
TCAAGAAGAGCAGGCAGACGCCCACAGCACCTTGGCCAAAATTAGAGTTAAA
TTCAGTAGAAGTGCGGATGCGCCTGCTTACCAGCAGGGCCAGAACCAACTGT
ACAATGAACTGAATCTCGGGCGCCGAGAAGAGTATGACGTCCTCGATAAGCG
GAGGGGTAGGGATCCTGAAATGGGTGGGAAGCCAAGAAGAAAAAACCCCCA
GGAAGGACTGTATAACGAACTTCAGAAGGACAAGATGGCAGAGGCCTACTCT
GAGATTGGCATGAAAGGCGAACGACGGCGCGGTAAAGGTCATGACGGGCTG
TACCAGGGCCTGTCCACAGCGACGAAGGACACTTACGACGCCCTGCACATG
CAGGCACTCCCCCCCAGGTGA
```

Namely, the nucleotide sequence, which encodes the sequence named also as SFG.iC9-2A-(NE97)B7-H3.CAR-ΔCD34.CD8a.CD28.4-1BBζ comprises the following sequences:

```
An inducible Caspase 9 (iC9):
  (SEQ ID NO: 40)
ATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTTTAT
GCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGT
GCCACTGGGCACCCAGGCATCATCCCACCACATGCCACTCTCGTCTTCGATG
TGGAGCTTCTAAAACTGGAATCTGGCGGTGGATCCGGAGTCGACGGATTTGG
TGATGTCGGTGCTCTTGAGAGTTTGAGGGGAAATGCAGATTTGGCTTACATC
CTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGA
AGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGG
ACCACGGTGCTCTGGACTGCTGCGTGGTGGTCATTCTCTCTCACGGCTGTCA
GGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGC
CTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTG
GCAGTAACCCCGAGCCAGATGCCACCCCGTTCCAGGAAGGTTTGAGGACCT
TCGACCAGCTGGACGCCATATCTAGTTTGCCCACACCCAGTGACATCTTTGT
GTCCTACTCTACTTTCCCAGGTTTTGTTTCCTGGAGGGACCCCAAGAGTGGC
TCCTGGTACGTTGAGACCCTGGACGACATCTTTGAGCAGTGGGCTCACTCTG
AAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTT
TAAAACATCAGCTAGC (GenBank ID MW218436.1),
which is linked by a first linker AGAGCC to:
2A self-cleaving peptides T2A
  (SEQ ID NO: 41)
GAGGGCAGGGGAAGTCTTCTAACATGCGGGGACGTGGAGGAAAATCCCGGG
CCC (nucleotide ID NO: NC_043231.1)
A Signal peptide
  (SEQ ID NO: 42)
ATGGAGTTTGGGCTCTCCTGGCTCTTCCTGGTCGCGATTCTGAAGGGGGTCC
AGTGTTCACGA (nucleotide ID NO: AB776838.1)
NE97 VL
  (SEQ ID NO: 29)
GACATCCAGATGACTCAGTCTCCAGCCTCCCTATCTGCATCTGTGGGAGAAA
CTGTCACCATCACATGTCGAGCAAGTGAGATTATTTACAGTTATTTAGCATGG
TATCAGCAGAAACAGGGAAAATCTCCTCAGCTCCTGGTCTATAATGCAAAAAC
CTTAGTAGAAGGTGTGCCATCAAGGTTCAGTGGCAGTGGATCAGGCACACAG
TTTTCTCTGAAGATCAACAGCCTGCAGCCTGAAGATTTTGGGAGTTATTACTG
TCAACATCATTATGGTACTCCTCCATACACGTTCGGAGGGGGGACCAAGCTG
GAAATAAAA
Flex
  (SEQ ID NO: 43)
GGCGGAGGTTCAGGCGGAGGAGGG
NE97 VH:
  (SEQ ID NO: 30)
GAGGTGCAGCTGGTGGAGTCTGGGGGAGACTTAGTGAAGCCTGGAGGGTCC
CTGAAACTCTCCTGTGCAGCCTCTGGATTCACTTTCAGTAGCTATGGCATGTC
TTGGGTTCGCCAGACTCCAGACAAGAGGCTGGAGTGGGTCGCAACCATTAAT
AGTGGTGGTAGTTACATCTACTATCCAGACAGTGTGAAGGGGCGATTCACCA
TCTCCAGAGACAATGCCGAGAACACCCTGTACCTGCAAATGAGCAGTCTGAA
GTCTGAAGACACAGCCATGTATTACTGTGCAAGACATGAAGGGTTACCCCTT
GACTACTGGGGCCAAGGCACCACTCTCACAGTCTCCTCA
Link (connection sequence)
GCATGC
ΔCD34
(SEQ ID NO: 44)
GAACTTCCTACTCAGGGGACTTTCTCAAACGTTAGCACAAACGTAAGT
(nucleotide ID NO AB238231.1)
Hinge Spacer-CD8α
  (SEQ ID NO: 45)
CCCGCCCCAAGACCCCCCACACCTGCGCCGACCATTGCTTCTCAACC
CCTGAGTTTGAGACCCGAGGCCTGCCGGCCAGCTGCCGGGGGGCCGTGC
ATACAAGAGGACTCGATTTCGCT
(nucleotide ID NO: M12828.1
CD8a TM
  (SEQ ID NO: 46)
TGCGACATCTACATCTGGGCTCCCCTCGCTGGCACCTGTGGGGTGCT
GCTGCTGTCACTCGTGATCACC
(nucleotide ID NO: NM_001768.6)
CD8a cytoplasmic (CD8a cyto):
  (SEQ ID NO: 47)
CTTTATTGCAACCATCGAAAC
(nucleotide ID NO: NM_001768.6)
Link (connection sequence)
GAATTC
CD28 cytoplasmic sequence:
  (SEQ ID NO: 48)
AGAAGTAAACGGTCAAGGCTTCTGCACAGCGATTATATGAATATGACA
CCAAGAAGACCTGGTCCAACCCGGAAACACTATCAGCCCTACGCGCCCCCTA
GAGACTTCGCAGCATACCGCTCT
(nucleotide ID NO: AF222341.1)
4-1BB sequence:
  (SEQ ID NO: 49)
AAGAGAGGGAGAAAAAAATTGCTCTATATTTTTAAACAACCATTTATGA
GGCCCGTACAGACAACTCAGGAAGAGGATGGCTGTAGTTGCCGCTTCCCAG
AGGAGGAGGAAGGAGGCTGCGAGTTG
(nucleotide ID NO: U03397.1)
CD3-Zeta chain:
(SEQ ID NO: 50)
AGAGTTAAATTCAGTAGAAGTGCGGATGCGCCTGCTTACCAGCAGGG
CCAGAACCAACTGTACAATGAACTGAATCTCGGGCGCCGAGAAGAGTATGAC
GTCCTCGATAAGCGGAGGGGTAGGGATCCTGAAATGGGTGGGAAGCCAAGA
AGAAAAAACCCCCAGGAAGGACTGTATAACGAACTTCAGAAGGACAAGATGG
CAGAGGCCTACTCTGAGATTGGCATGAAAGGCGAACGACGGCGCGGTAAAG
GTCATGACGGGCTGTACCAGGGCCTGTCCACAGCGACGAAGGACACTTACG
ACGCCCTGCACATGCAGGCACTCCCCCCCAGGTGA
(nucleotide ID NO: J04132.1)
```

The nucleotide sequence, which encodes the sequence named also as SFG.iC9-2A-(NE97)B7-H3.CAR-ΔCD34.CD8a.CD28.OX40ζ comprises the following sequences:

```
An inducible Caspase 9 (iC9):
  (SEQ ID NO: 40)
ATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTTTAT
GCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGT
GCCACTGGGCACCCAGGCATCATCCCACCACATGCCACTCTCGTCTTCGATG
TGGAGCTTCTAAAACTGGAATCTGGCGGTGGATCCGGAGTCGACGGATTTGG
TGATGTCGGTGCTCTTGAGAGTTTGAGGGGAAATGCAGATTTGGCTTACATC
CTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGA
AGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGG
ACCACGGTGCTCTGGACTGCTGCGTGGTGGTCATTCTCTCTCACGGCTGTCA
GGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGC
CTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTG
GCAGTAACCCCGAGCCAGATGCCACCCCGTTCCAGGAAGGTTTGAGGACCT
TCGACCAGCTGGACGCCATATCTAGTTTGCCCACACCCAGTGACATCTTTGT
GTCCTACTCTACTTTCCCAGGTTTTGTTTCCTGGAGGGACCCCAAGAGTGGC
TCCTGGTACGTTGAGACCCTGGACGACATCTTTGAGCAGTGGGCTCACTCTG
AAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTT
TAAAACATCAGCTAGC (GenBank ID MW218436.1), which is
linked by a first linker AGAGCC to:
2A self-cleaving peptides T2A
  (SEQ ID NO: 41)
GAGGGCAGGGGAAGTCTTCTAACATGCGGGGACGTGGAGGAAAATCCCGGG
CCC
(nucleotide ID NO: NC_043231.1)
A Signal peptide
  (SEQ ID NO: 42)
ATGGAGTTTGGGCTCTCCTGGCTCTTCCTGGTCGCGATTCTGAAGGGGGTCC
AGTGTTCACGA
(nucleotide ID NO: AB776838.1)
NE97 VL
  (SEQ ID NO: 29)
GACATCCAGATGACTCAGTCTCCAGCCTCCCTATCTGCATCTGTGGGAGAAA
CTGTCACCATCACATGTCGAGCAAGTGAGATTATTTACAGTTATTTAGCATGG
TATCAGCAGAAACAGGGAAAATCTCCTCAGCTCCTGGTCTATAATGCAAAAAC
CTTAGTAGAAGGTGTGCCATCAAGGTTCAGTGGCAGTGGATCAGGCACACAG
TTTTCTCTGAAGATCAACAGCCTGCAGCCTGAAGATTTTGGGAGTTATTACTG
TCAACATCATTATGGTACTCCTCCATACACGTTCGGAGGGGGGACCAAGCTG
GAAATAAAA
Flex
  (SEQ ID NO: 43)
GGCGGAGGTTCAGGCGGAGGAGGG
NE97 VH:
(SEQ ID NO: 30)
GAGGTGCAGCTGGTGGAGTCTGGGGGAGACTTAGTGAAGCCTGGAGGGTCC
CTGAAACTCTCCTGTGCAGCCTCTGGATTCACTTTCAGTAGCTATGGCATGTC
TTGGGTTCGCCAGACTCCAGACAAGAGGCTGGAGTGGGTCGCAACCATTAAT
AGTGGTGGTAGTTACATCTACTATCCAGACAGTGTGAAGGGGCGATTCACCA
TCTCCAGAGACAATGCCGAGAACACCCTGTACCTGCAAATGAGCAGTCTGAA
GTCTGAAGACACAGCCATGTATTACTGTGCAAGACATGAAGGGTTACCCCTT
GACTACTGGGGCCAAGGCACCACTCTCACAGTCTCCTCA
Link (connection sequence)
GCATGC
ΔCD34
(SEQ ID NO: 44)
GAACTTCCTACTCAGGGGACTTTCTCAAACGTTAGCACAAACGTAAGT
(nucleotide ID NO AB238231.1)
Hinge Spacer-CD8α
  (SEQ ID NO: 45)
CCCGCCCCAAGACCCCCCACACCTGCGCCGACCATTGCTTCTCAACC
CCTGAGTTTGAGACCCGAGGCCTGCCGGCCAGCTGCCGGCGGGGCCGTGC
ATACAAGAGGACTCGATTTCGCT
(nucleotide ID NO: M12828.1
CD8aTM
  (SEQ ID NO: 46)
TGCGACATCTACATCTGGGCTCCCCTCGCTGGCACCTGTGGGGTGCT
GCTGCTGTCACTCGTGATCACC
(nucleotide ID NO: NM_001768.6 )
CD8a cytoplasmic (CD8a cyto):
  (SEQ ID NO: 47)
CTTTATTGCAACCATOGAAAC
(nucleotide ID NO: NM_001768.6)
Link (connection sequence)
GAATTC
CD28 cytoplasmic sequence:
  (SEQ ID NO: 48)
AGAAGTAAACGGTCAAGGCTTCTGCACAGCGATTATATGAATATGACA
CCAAGAAGACCTGGTCCAACCCGGAAACACTATCAGCCCTACGCGCCCCCTA
GAGACTTCGCAGCATACCGCTCT
(nucleotide ID NO: AF222341.1)
OX40 sequence
  (SEQ ID NO: 51)
CGCGATCAAAGACTCCCGCCCGATGCCCACAAACCCCCTGGGGGGGGCAGC
TTTAGGACACCCATTCAAGAAGAGCAGGCAGACGCCCACAGCACCTTGGCCA
AAATT
(nucleotide ID NO: NM_003327.3)
CD3-Zeta chain:
(SEQ ID NO: 50)
AGAGTTAAATTCAGTAGAAGTGCGGATGCGCCTGCTTACCAGCAGGG
CCAGAACCAACTGTACAATGAACTGAATCTCGGGCGCCGAGAAGAGTATGAC
GTCCTCGATAAGCGGAGGGGTAGGGATCCTGAAATGGGTGGGAAGCCAAGA
AGAAAAAACCCCCAGGAAGGACTGTATAACGAACTTCAGAAGGACAAGATGG
CAGAGGCCTACTCTGAGATTGGCATGAAAGGCGAACGACGGCGCGGTAAAG
GTCATGACGGGCTGTACCAGGGCCTGTCCACAGCGACGAAGGACACTTACG
ACGCCCTGCACATGCAGGCACTCCCCCCCAGGTGA
(nucleotide ID NO: J04132.1).
```

The present invention concerns also a vector comprising the nucleotide sequence as defined above, wherein said vector is a DNA vector, a RNA vector, a plasmid, a lentivirus vector, adenoviral vector, retrovirus vector, such as γ-retroviral vector, or non-viral vector.

In addition, the present invention concerns a cell, such as T cell, such as alfa/beta and gamma/delta T cell, NK cells, NK-T cells, comprising the anti-B7-H3 chimeric antigen receptor according to the above and/or the vector or plasmid according to the above.

According to the present invention, the cell can further comprise a suicide gene inducible amino acid sequence such as a chimeric Caspase-9 polypeptide or a herpes simplex virus thymidine kinase (HSV-TK) as a safety switch.

According to the present invention, the chimeric Caspase-9 polypeptide can comprise or consist of:

an inducible Caspase 9 (iC9):
(SEQ ID NO: 52)
MLEGVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFDVELLK
LESGGGSGVDGFGDVGALESLRGNADLAYILSMEPCGHCLIINNVNFCRESGLR
TRTGSNIDCEKLRRRFSSLHFMVEVKGDLTAKKMVLALLELAQQDHGALDCCVV
VILSHGCQASHLQFPGAVYGTDGCPVSVEKIVNIFNGTSCPSLGGKPKLFFIQAC
GGEQKDHGFEVASTSPEDESPGSNPEPDATPFQEGLRTFDQLDAISSLPTPSDIF
VSYSTFPGFVSWRDPKSGSWYVETLDDIFEQWAHSEDLQSLLLRVANAVSVKGI
YKQMPGCFNFLRKKLFFKTSAS
(GenBank ID MW218436.1 and Protein ID NO: QPB74035.1),
which is linked by a linker, such as RA, to:
2A self-cleaving peptides T2A
(SEQ ID NO: 53)
EGRGSLLTCGDVEENPGP
(nucleotide ID NO: NC_043231.1 and Protein ID NO: YP_009665206.1).

According to the present invention the cell can be obtained in culture conditions wherein both IL-7 and IL-15 are present, for example in the culture conditions of the activation step, transduction step and/or expansion step of the process for the preparation of said cell.

The present invention concerns also a pharmaceutical composition comprising the nucleotide sequence as defined above, or the vector according to the above, or the cell according to the above together with one or more excipients and/or adjuvants.

According to a further embodiment, the present invention concerns an anti B7-H3 chimeric antigen receptor according to the above, a nucleotide sequence according to the above, a vector according to the above, a cell according to the above, a pharmaceutical composition according to the above, for medical use.

In addition, the present invention concerns, an anti B7-H3 chimeric antigen receptor according to the above, a nucleotide sequence according to the above, a vector according to the above, a cell according to the above, a pharmaceutical composition according to the above, for use in the treatment of hematologic malignancies, such as for example Chronic Myeloid Leukemia (CML), Myelodysplastic syndromes (MDS), Acute Myeloid Leukemia (AML), Chronic lymphocytic leukemia (CLL), B cell Acute lymphoblastic leukemia (B-ALL), T cell Acute lymphoblastic leukemia (T-ALL), lymphomas (Non-Hodgkin's Lymphoma or Hodgkin's Lymphoma), Multiple Myeloma, and solid B7H3+ tumour, such as for example Neuroblastoma, retinoblastoma, sarcoma, Ewing's sarcoma, rhabdomyosarcoma, Osteosarcoma, Desmoplastic Small Round Cell Tumors (DSRCT), pancreatic cancer, Colon cancer, breast cancers, germinal carcinoma, lung carcinoma, liver carcinoma, kidney carcinoma, melanoma and brain tumors such as medulloblastoma and glioblastoma, and autoimmune diseases.

The present invention concerns also the B7-H3 CAR according to the above, the nucleotide sequence according to the above, the vector according to the above, the cell according to the above, the pharmaceutical composition according to the above, for use in the treatment of autoimmune diseases.

Autoimmune diseases are a class of common, complex, inflammatory disorders including systemic lupus erythematosus (SLE), rheumatoid arthritis (RA), multiple sclerosis (MS), Sjögren's syndrome (SS), and ankylosing spondylitis (AS).

The anti B7-H3 chimeric antigen receptor, nucleotide sequence, vector, cell, pharmaceutical composition according to the present invention can be advantageously administered by systemic administration, also in the treatment of brain tumors.

The present invention now will be described by an illustrative, but not limitative way, according to the preferred embodiments thereof, with particular reference to the examples and the enclosed drawings, wherein:

FIG. 1 shows four B7-H3.CAR SFG clinical grade “third” generation of retrovirus vector. (A-B) The scFv of B7H3 (Ne97) was cloned in frame with CD8aTM, CD28 cytoplasmic moiety, and a second costimulatory domain represented by either 4-1BB (A) or OX40 (B), as well as the signaling domain CD3-zeta chain (ζ). As a trackable marker, ΔCD34 was added. (C-D) The scFv of B7H3 (M5B14, object of the twinned patent) was cloned in frame with CD8aTM, CD28 cytoplasmic moiety, and a second costimulatory domain represented by either 4-1BB (C) or OX40 (D), as well as the signaling domain CD3-zeta chain (ζ). As a trackable marker, ΔCD34 was added.

FIG. 2 shows that B7-H3.CAR T cells with CD28.OX40 or CD28.4-1BB costimulation exhibit high transduction level. (A) Flow-cytometry analyses in a representative donor showing chimeric antigen receptor (CAR) expression by detection of membrane ΔCD34 in non-transduced (NT) T cells (negative control; left panel), and T cells genetically modified with NE97.B7-H3.CAR-28.4-1BBζ, NE97.B7-H3.CAR-28.OX40ζ, M5B14.B7-H3.CAR-28.4-1BBζ and M5B14.B7-H3.CAR-28.OX40ζ. growth in IL2 (from the second to the fifth square panel, respectively). (B) Percentage of CAR+CD3+ T-cells at day 15 in NT T cells (black dots texture bar), NE97.B7-H3.CAR T-cells (white bars) and M5B14.B7-H3.CAR T-cells (black bar). (C) Violin plot of Median Fluorescence Intensity (MFI) analysis of CAR+CD3+ T-cells expression on NT T-cells (left), NE97.B7-H3.CAR-28.4-1BBζ and NE97.B7-H3.CAR-28.OX40ζ T cells (middle), M5B14.B7-H3.CAR-28.4-1BBζ and M5B14.B7-H3.CAR-28.OX40ζ T-cells (right). Data from 5 HDs are expressed as average±SD. * p-values≤0.05; ** p-values≤0.01

FIG. 3 shows that B7-H3.CAR T cells with CD28.OX40 or CD28.4-1BB costimulation exhibit similar in vitro proliferation and safety profile upon cytokine stimulation. (A) Fold expansion of NT T-cells (black dots on dashed line), NE97.B7-H3.CAR-28.4-1BBζ T-cells (empty dots), NE97.B7-H3.CAR-28.OX40ζ T-cells (empty triangles), M5B14.B7-H3.CAR-28.4-1BBζ T-cells (black dots) and M5B14.B7-H3.CAR-28.OX40ζ T-cells (black triangle) grown in IL2. (B) TCR Vβ repertoire analysis of NT and B7-H3.CAR T-cells grown in the presence of IL2 at Day +15 showing a regular polyclonal Vβ repertoire, without any preferential oligo or monoclonal selection. (C) Evaluation of percentage of live (Annexin-V−/7AAD−) NT or B7-H3.CAR T-cells grown in the presence of IL2 and exposed to 10 nM AP1903 for 48 hours. (D) The Expression of Vector Copy Number of B7-H3.CAR T-cells, at day +15 was below 12, (range 1.1-11.5). Data from six healthy donors (HDs) are expressed as average±SD. ****p-value=<0,0001.

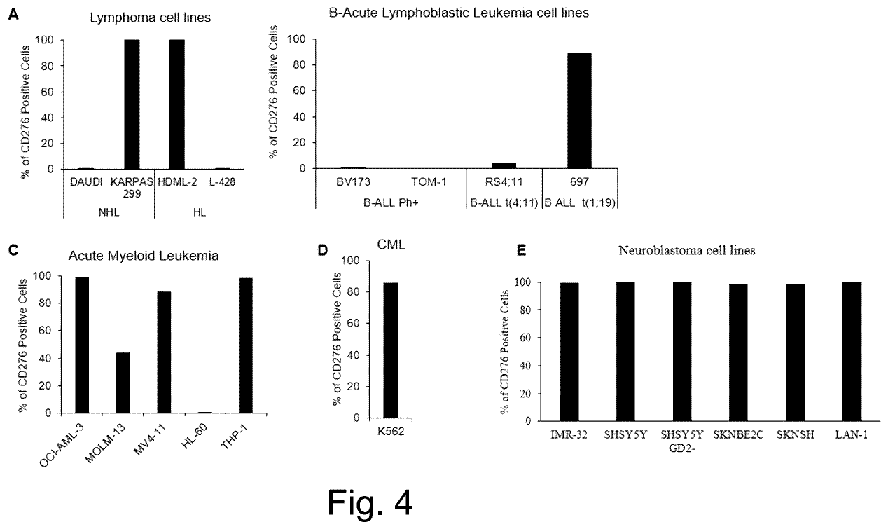

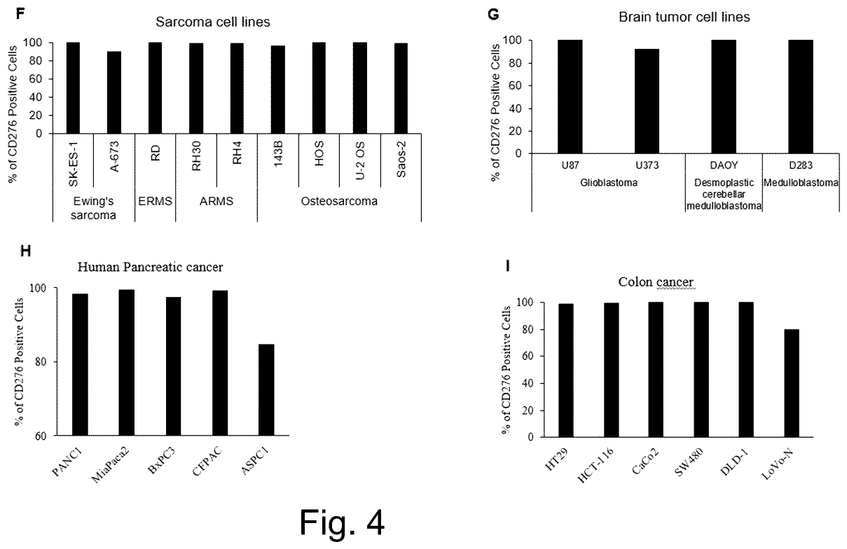

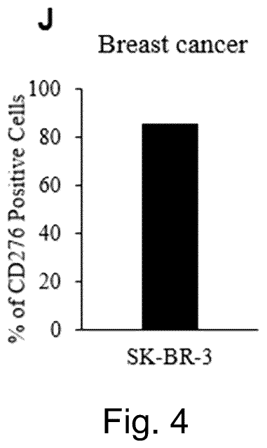

FIG. 4 shows B7-H3 (CD276) Expression in solid and hematological tumors cell lines. The expression of CD276 (B7-H3) antigen was evaluated, by flow cytometry. The protein B7-H3 is widely expressed in (A) two out of four lymphoma cell lines; (B) one in four B-acute lymphoblastic leukemia cell lines; (C) four out of five Acute Myeloid Leukaemia; (D) one chronic myeloid leukaemia; (E) all evaluated neuroblastoma cell lines; (F) all evaluated sarcoma cell lines (specifically Ewing's sarcoma, Embryonal rhabdomyosarcoma (ERMS), alveolar Rhabdomyosarcoma (ARMS) and Osteosarcoma cell lines); (G) all evaluated Brain tumours (specifically Glioblastoma and MB cell lines); (H) all evaluated pancreatic cancer cell lines; (I) all evaluated Colon cancer cell lines and (J) Breast cancer cell lines.

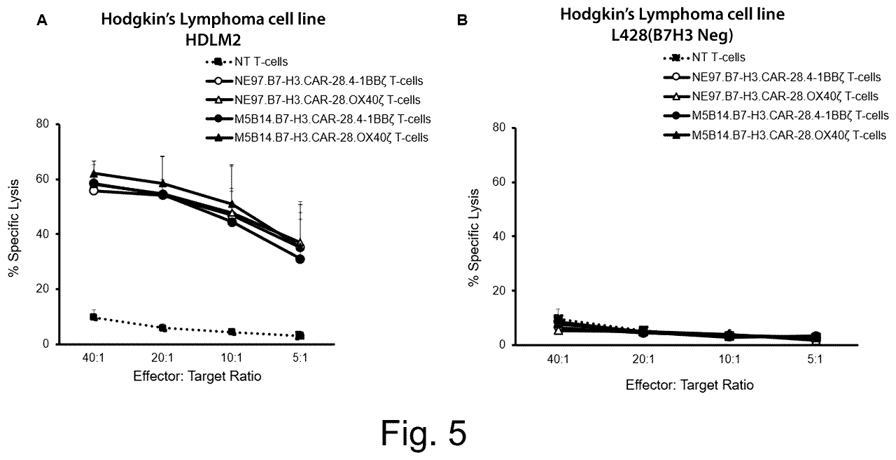

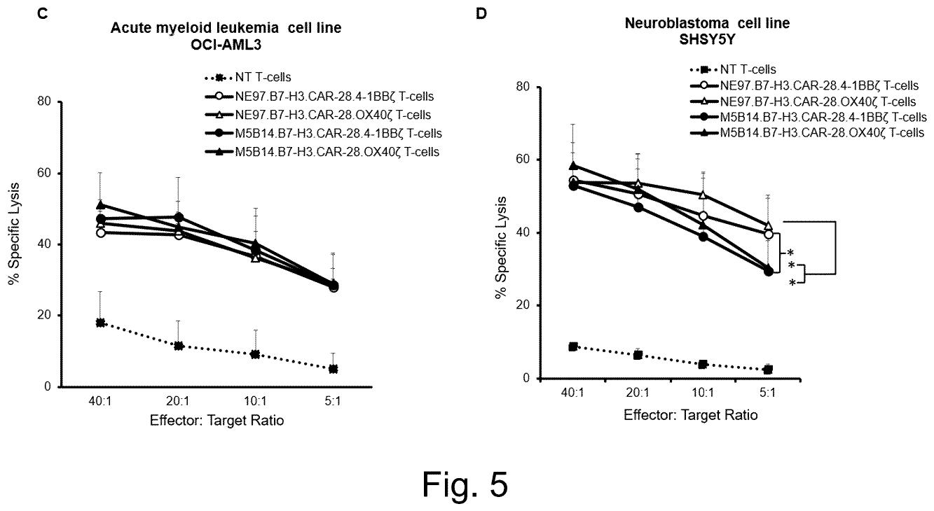

FIG. 5 shows that both scFv in B7-H3.CAR T cells (NE97 and M5B14), expressing either CD28.4-1BB or CD28.OX40 costimulatory domains, show comparable short-term cytotoxic effect in vitro experiment. In vitro ⁵¹Cr release assay showing that NE97.B7-H3.CAR-28.4-1BBζ T-cells (solid line with empty dots), NE97.B7-H3.CAR-28.OX40ζ T-cells (solid line with empty triangles), M5B14.B7-H3.CAR-28.4-1BBζ T-cells (solid line with black dots) and M5B14.B7-H3.CAR-28.OX40ζ T-cells (solid line with black triangle) exert cytolytic activity against B7-H3+ Hodgkin Lymphomas HDLM-2 (A), and Acute Myeloid Leukemia OCI-AML3 (C) but not against B7-H3 negative Hodgkin Lymphomas L428. NT T cells (dotted line with black square) were applied as negative control of the experiment (B). Note that, at a very low E:T ratio (5:1), NE97.B7-H3.CAR-28.OX40ζ CAR T-cells kill the SHSY5Y NB cell line with significant higher efficiency respect to M5B14.B7-H3.CAR-28.4-1BBζ or M5B14.B7-H3.CAR-28.OX40ζ T cells (D). Data from six healthy donors (HDs) are expressed as average±SD. * p-value≤0.05.

FIG. 6 shows that long-term co-culture of both NE97 and M5B14 scFv B7-H3.CAR T cells (NE97 and M5B14), expressing either CD28.4-1BB or CD28.OX40 costimulatory domains, confirm their specific cytotoxic potency against different B7-H3+ tumor cell lines derived from extracranial neoplasia. Average representation of remaining tumor cells, after 6 days-coculture at the ratio E/T 1:1 with NT T-cells, NE97.B7-H3.CAR-28.4-1BBζ T-cells, NE97.B7-H3.CAR-28.OX40ζ T-cells, M5B14.B7-H3.CAR-28.4-1BBζ T-cells and M5B14.B7-H3.CAR-28.OX40ζ T-cells, growth in IL2. In (A-B) Neuroblastoma cell lines; (C) Ewing sarcoma A673 cell line; (D) ERMS RD cell line; (E) Hodgkin Lymphoma HDML-2 cell line; (F) Acute Myeloid Leukemia OCI-AML3; (G) Acute monoblastic/monocytic leukemia; (H) and Pre-B lymphoblastic leukemia 697. Data from six healthy donors (HDs) are expressed as average±SD. * p-value≤0.05; ** p-value≤0.01; *** p-value≤0.001 and ****≤0.0001.

FIG. 7 shows that long-term co-culture of both NE97 and M5B14 scFv B7-H3.CAR T cells (NE97 and M5B14), expressing either CD28.4-1BB or CD28.OX40 costimulatory domains confirm their potent specific cytotoxic potency against B7-H3+ brain tumor cell lines. Average representation of remaining tumor cells, after 6 days-coculture at the ratio E/T 1:1 with NT T-cells, NE97.B7-H3.CAR-28.4-1BBζ T-cells, NE97.B7-H3.CAR-28.OX40ζ T-cells, M5B14.B7-H3.CAR-28.4-1BBζ T-cells and M5B14.B7-H3.CAR-28.OX40ζ T-cells, growth in IL2. In (A-B) Medulloblastoma cell lines D283 and DAOY; (C-D) in glioblastoma cell lines U87 and U373. Data from six healthy donors (HDs) are expressed as average±SD. * p-values≤0.05; ** p-values≤0.01; *** p-values≤0.001 and ****≤0.0001.

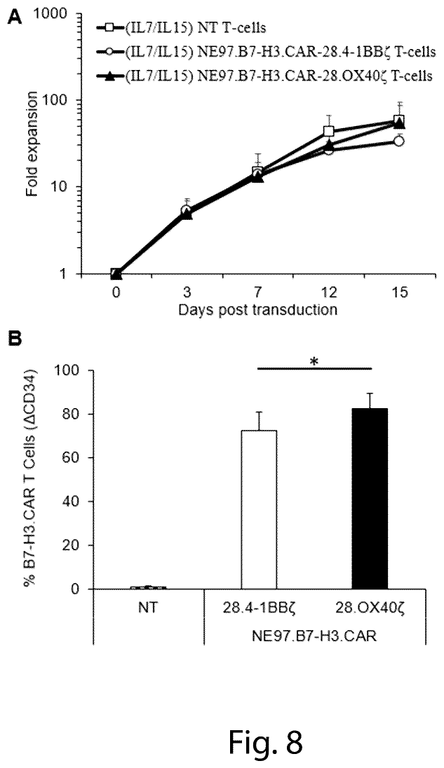

FIG. 8 shows that IL7/IL15 used in culture conditions did not influence the kinetic expansion of NE97.B7-H3.CAR Tcells. (A) Fold expansion n of NT T-cells (White Square), NE97.B7-H3.CAR-28.4-1BBζ T-cells (empty dots), NE97.B7-H3.CAR-28.OX40ζ T-cells (black triangles), grown in IL7/IL15. (B) Percentage of CAR+CD3+ T-cells at day 15 in NT T-cells (white bar with black dots), NE97.B7-H3.CAR-28.4-1BBζ T-cells (white bars) and NE97.B7-H3.CAR-28.OX40ζ T-cells. Data from six healthy donors (HDs) are expressed as average±SD. * p-value≤0.05.

FIG. 9 shows that cytokines used in culture conditions significantly improve in vitro killing activity of NE97.B7-H3.CAR T-cells expressing either CD28.4-1BB or CD28.OX40 costimulatory domains. Average representation of remaining tumor cells of several tumor cell lines as the MB cell line DAOY (A); the neuroblastoma cell line SHSY5Y (B); the Osteosarcoma (OS) cell lines (C-E): 143B (C), HOS (D) and 02-OS (E); the ARMS cell lines (F-G): RH30 (F) and RH41 (G); the ERMS RD (H), the Ewing's sarcoma A673 (I) and SK-ES-1 (J) and the Non-Hodjkin Lymphoma Karpas 299 (K), after 6 days co-culture, at the ratio E/T 1:1, with NT T-cells, NE97.B7-H3.CAR-28.4-1BBζ T-cells, NE97.B7-H3.CAR-28.OX40ζ T-cells growth in IL2 (white bars) (A) or IL7/IL15 (B-K) (black bars). Data from six healthy donors (HDs) are expressed as average±SD. * p-value≤0.05; ** p-value≤0.01; *** p-value≤0.001 and ****≤0.0001.

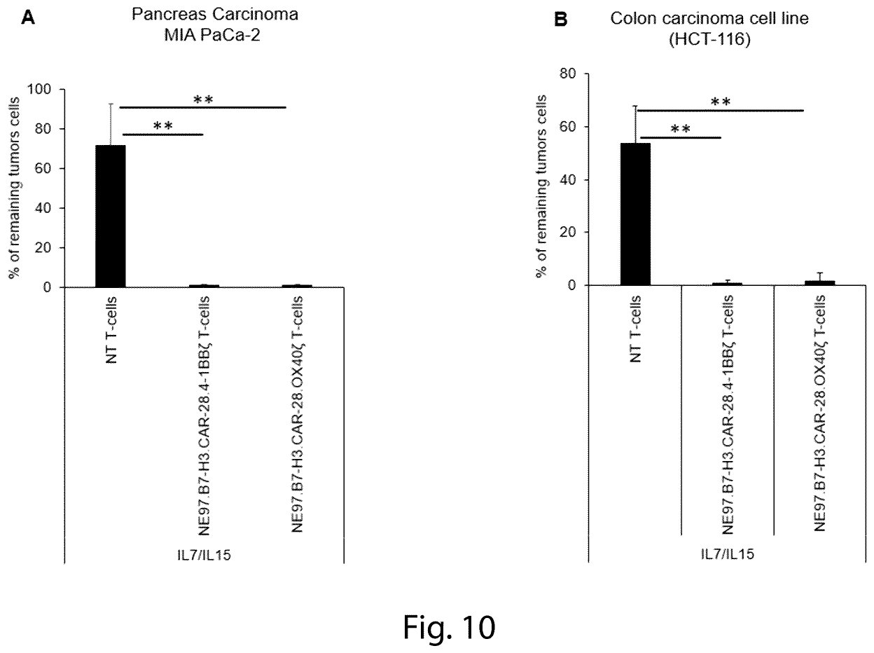

FIG. 10 shows that both (IL7/IL15) NE97.B7-H3.CAR T-cells, expressing either CD28.4-1BB or CD28.OX40 costimulatory domains, kill very efficiently also adult solid tumor cell lines. Average representation of remaining tumor cells of several tumor cell lines as the pancreatic carcinoma cell line MIA PaCa-2(A) and the colon carcinoma cell line HCT-116 (B). Data from four healthy donors (HDs) are expressed as average±SD. * p-value≤0.05; ** p-value≤0.01.

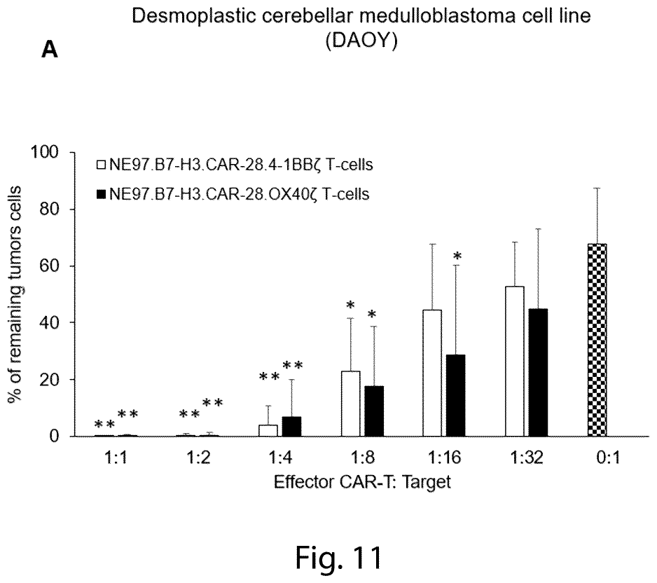

FIG. 11 shows in vitro long-term co-culture potency assay to evaluate functional activities of NE97.B7-H3.CAR T-cells. (A) Tumor cell growth after 7 days of co-culture at low E:T ratios with NT (chess bar), NE97.B7-H3.CAR-28.4-1 BBζ T-cells (white bar) and NE97.B7-H3.CAR-28.OX40ζ T-cells (black bar) grown in IL7/IL15. The B7-H3+MB DAOY tumor cell line was used as target. Data from 4 HDs are expressed as average±SD for (D); * p-value≤0.05; ** p-value≤0.01.

FIG. 12 shows that NE97.B7-H3.CAR-28.OX40 CAR T-cells produce higher CAR-T-derived cytokines respect to NE97.B7-H3.CAR-28.4-1BBζ T-cells when co-cultured with B7-H3+ tumor cell lines RH30 (A-D) or A673 (E-H). (A and E) GranB, (B and F) IFN-γ, (C and H) TNF-α, and (D and G) IL-2 production were analysed in supernatants collected 24-hours after tumor addition to the culture. Data from six healthy donors (HDs) are expressed as average±SD. * p-value≤0.05; ** p-value≤0.01; ***p-value≤0.001 and ****≤0.0001.

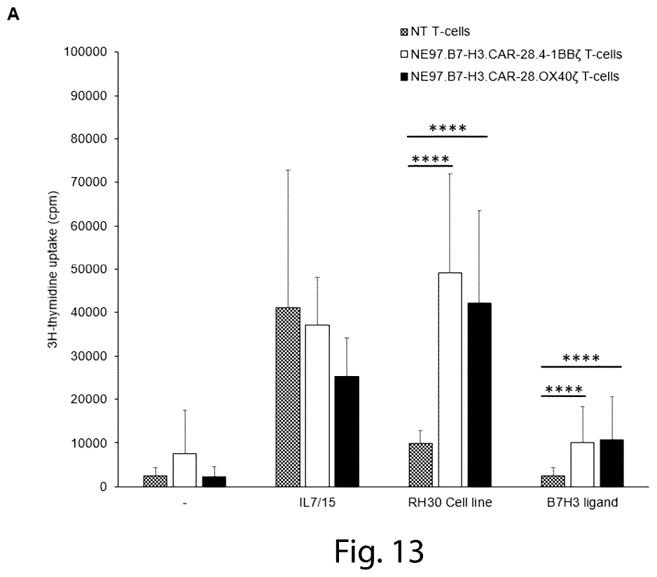

FIG. 13 shows that NE97.B7-H3.CAR T-cells proliferate specifically when co-cultured with B7-H3+ tumor cell line or upon activation by B7-H3 Ligand. (A) Proliferation assay based on 3H-thymidine incorporation of NT T-cells or NE97.B7-H3.CAR T-cells cells, stimulated for five days, with irradiated RH30 tumor cells (45 Gy), or the cytokine cocktail (IL7/IL15), or the B7-H3 ligand. Data represent results from 3 HDs. * p-value=<0.05, ** p-value=<0.01, *** p-value=<0.001, **** p-value=<0.0001.

FIG. 14 shows the results obtained with sarcoma mouse model to evaluate anti-tumor activity of NE97.B7-H3.CAR T-cells generated and expanded in the presence of IL7/IL15. A) The cartoon shows the in vivo xenograft immunodeficient mouse model, in which the ARMS cell lines RH30-GFP-FF-Luc cells were systemically infused in NSG mice. Effector cells were infused i.v. at the time of tumor establishment (Day 3), as assessed by IVIS Imaging. (B) Exemplificative IVIS Imaging of tumor growth from day +2 to end-of-experiment for mice treated with NT T-cells or NE97.B7-H3.CAR T-cells. (C) Average of tumor bioluminescence of xenograft mice treated with NT T-cells (dotted line), with NE97.B7-H3.CAR-28.4-1BBζ T-cells (dotted line with black dots) or with NE97.B7-H3.CAR-28.OX40ζ T-cells (solid line with black dots). (D) Kaplan-Meier survival curve (OS) analysis of tumor-bearing mice treated with NT (dotted line) with NE97.B7-H3.CAR-28.4-1BBζ T-cells line (dash-dotted line) or with NE97.B7-H3.CAR-28.OX40ζ T-cells (solid line). * P-value=<0.05.

FIG. 15 shows the results obtained with MB intracranial mouse model to evaluate the antitumor activity of NE97.B7-H3.CAR T-cells. A) The cartoon shows NSG mice engrafted with MB cell line D283.GFP-FF-luciferase in the brain, by stereotaxic system. Effector cells were infused i.v. at the time of tumor establishment (Day 14), as assessed by IVIS Imaging. Mice underwent periodical blood collections. (B) Exemplificative IVIS Imaging of tumor growth from day +14 to end-of-experiment for mice treated with NT T-cells or NE97.B7-H3.CAR T-cells. (C) Average of tumor bioluminescence of xenograft mice treated with NT T-cells (dotted line), with NE97.B7-H3.CAR-28.4-1BBζ T-cells (dotted line with black dots) or with NE97.B7-H3.CAR-28.OX40ζ T-cells (solid line with black dots). (D) Kaplan-Meier survival curve (OS) analysis of tumor-bearing mice treated with NT (dotted line) with NE97.B7-H3.CAR-28.4-1BBζ T-cells line (dash-dotted line) or with NE97.B7-H3.CAR-28.OX40ζ T-cells (solid line). (E) Average of circulating human CD45+CD3+ T-cells in mice treated with NT T-cells (dotted line with black dots), NE97.B7-H3.CAR-28.4-1BBζ T-cells (dotted line with Black Square) and NE97.B7-H3.CAR-28.OX40ζ T-cells (solid line with black triangle). (F) Violin plot of circulating human CD3+ CAR+ T-cells in mice treated with NT T-cells (white violin plot), NE97.B7-H3.CAR-28.4-1BBζ T-cells (white with black polka dots violin plot), and NE97.B7-H3.CAR-28.OX40ζ T-cells (black violin plot). * P-value=<0.05, ** p-value=<0.01, *** p-value=<0.001, **** p-value=<0.0001.

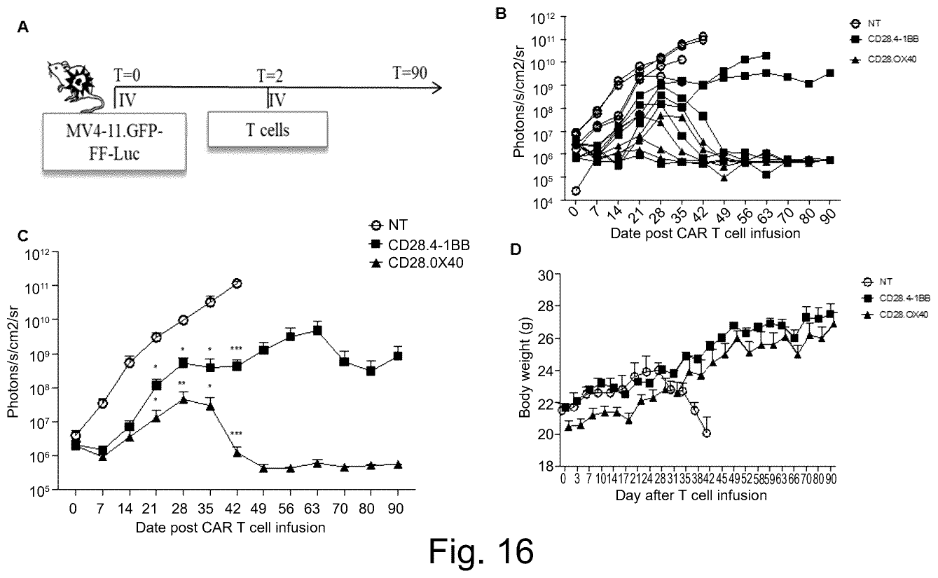

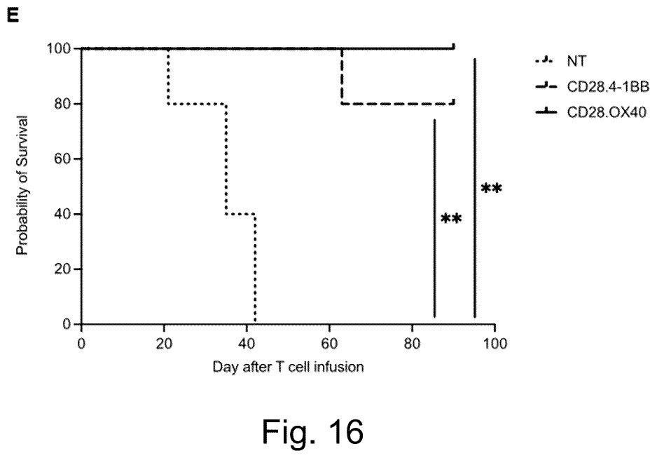

FIG. 16. AML murine model to evaluate anti-tumor activity of NE97.B7-H3.CAR T-cells. (A) The cartoon shows the design of the xenograft in vivo murine model, in which the AML cell line, MV4-11.GFP-FF-Luc, were systemically infused in NSG mice. T cells were infused i.v. at the time of tumor establishment (Day 2), which was assessed by IVIS Imaging. (B) Tumor bioluminescence of each mouse treated with NT T-cells (white circles), with NE97.B7-H3.CAR-28.4-1BBζ (CD28.4-1BB) T-cells (black squares) or with NE97.B7-H3.CAR-28.OX40ζ (CD28.OX40ζ) T-cells (black triangles). (C) Average+SEM of tumor bioluminescence of AML engrafted mice treated with NT T-cells (white circles), with NE97.B7-H3.CAR-28.4-1BBζ (CD28.4-1BB) T-cells (black squares) or with NE97.B7-H3.CAR-28.OX40ζ (CD28.OX40ζ) T-cells (black triangles). * P-value=<0.05, ** p-value=<0.01, *** p-value=<0.001, **** p-value=<0.0001. (D) Treatment of AML engrafted mice with NE97.B7-H3.CAR T-cells did not induce any Graft versus Host Disease (GvHD) effect. The graph show average±SEM of body weight of AML engrafted mice treated with NT T-cells (white circles), with NE97.B7-H3.CAR-28.4-1BBζ (CD28.4-1BB) T-cells (black squares) or with NE97.B7-H3.CAR-28.OX40ζ (CD28.OX40ζ) T-cells (black triangles). (E) Kaplan-Meier survival curve (OS) analysis of AML tumor-bearing mice treated with NT (dotted line) with NE97.B7-H3.CAR-28.4-1BBζ (CD28.4-1BB) T-cells line (dash-dotted line) or with NE97.B7-H3.CAR-28.OX40ζ (CD28.OX40ζ) T-cells (solid line). ** p-value=<0.01.

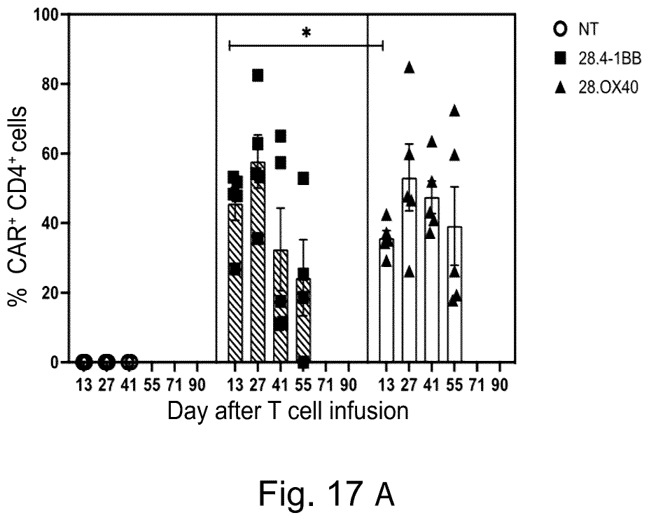

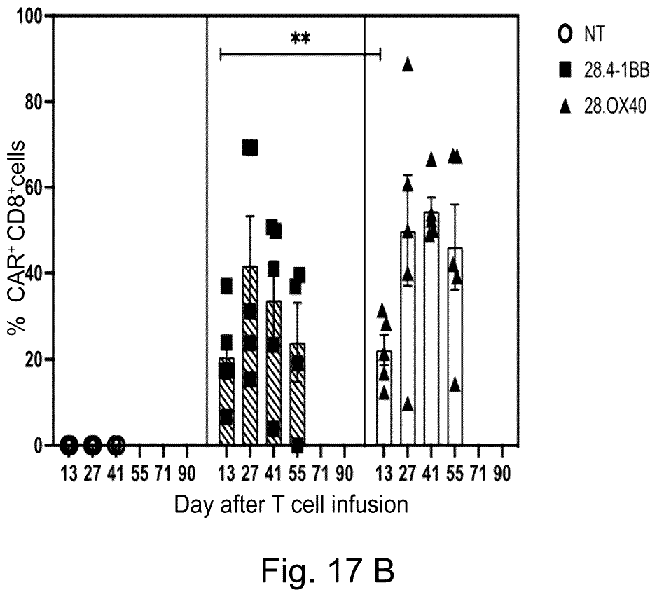

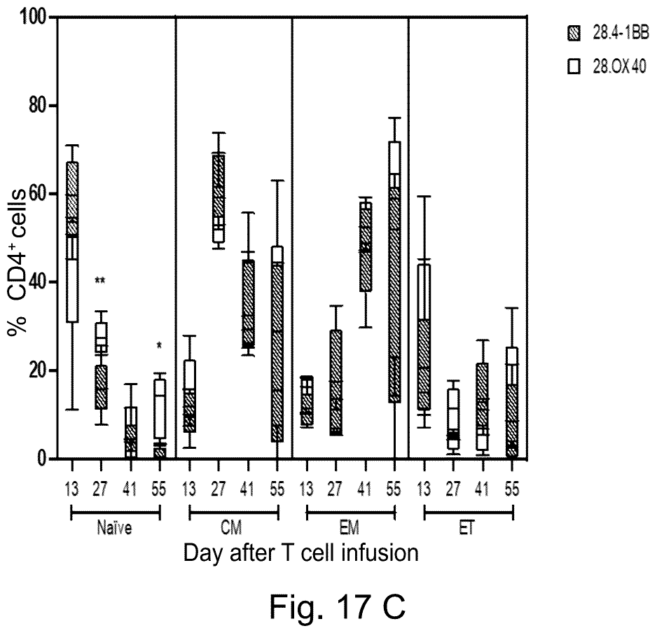

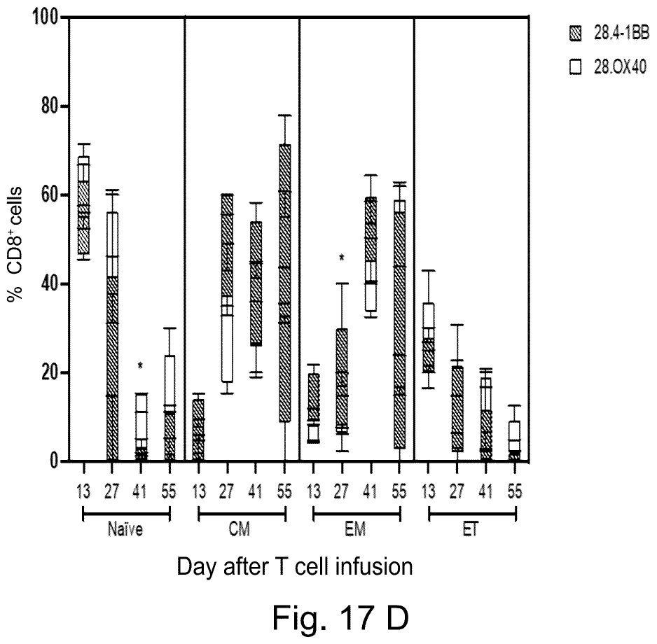

FIG. 17. Immunophenotype of AML engrafted mice treated with NT and NE97.B7-H3.CAR T-cells. CAR T population was detected in the peripheral blood of mice treated with both B7-H3.CARs until day 55. Both CD4+(A) and CD8+(B) subpopulations expanded in vivo. A significant difference in CAR T cell expansion between NE97.B7-H3.CAR-28.4-1BBζ (28.4-1BB) and NE97.B7-H3.CAR-28.OX40ζ (28.OX40ζ) was detected in both CD4 (A, p=0,034) and CD8 (B, p=0,005) subsets at day 13. Both CD4 and CD8 CAR T cells showed functional maturation after tumor engagement. Furthermore, a significant difference was detected in the naïve CD4 subpopulation between NE97.B7-H3.CAR-28.4-1BBζ (28.4-1BB) and NE97.B7-H3.CAR-28.OX40ζ (28.OX40ζ) at day 27 and day 55 (C) as well as in the naïve (day 41) and effector memory (day 27) CD8 subpopulation (D).

### EXAMPLE 1: PREPARATION OF B7-H3-SPECIFIC CHIMERIC ANTIGEN RECEPTOR (B7-H3 CAR) EFFECTOR CELLS AND STUDY OF THE EFFECTS THEREOF IN THE TREATMENT OF B7-H3 (CD276) POSITIVE TUMORS

### Material and Methods

The geographical origin and the code of the cell lines that have been used in the experiments are shown in table 5 below.

TABLE 5

| Cell | Distributor/Registered | | |
|---|---|---|---|
| line | Office | Product code | Country of origin |
| HDML- | DSMZ/ | ACC 17 | Germany |
| 2 | Inhoffenstraße 7B 38124 | | |
| | Braunschweig GERMANY | | |
| L428 | DSMZ/ | ACC 197 | Germany |
| | Inhoffenstraße 7B 38124 | | |
| | Braunschweig GERMANY | | |
| BV173 | DSMZ | ACC 20 | Germany |
| | Inhoffenstraße 7B/38124 | | |
| | Braunschweig GERMANY | | |
| TOM-1 | DSMZ | ACC 578 | Germany |
| | Inhoffenstraße 7B/38124 | | |
| | Braunschweig GERMANY | | |
| 697 | DSMZ | ACC 42 | Germany |
| | Inhoffenstraße 7B/38124 | | |
| | Braunschweig GERMANY | | |
| RS4;11 | DSMZ | ACC 508 | Germany |
| | Inhoffenstraße 7B/38124 | | |
| | Braunschweig GERMANY | | |
| DAUDI | LGC Standards S.r.I-ATCC./ | CCL-213 | Italy |
| | Via Carducci, 39 20099 | | |
| | Sesto San Giovanni (MI) Italy | | |
| Karpas | Sigma Aldrich SRL/ | 06072604-1VL | Italy |
| 299 | Via Monte Rosa 93 20149 | | |
| | Milano | | |
| | Italia | | |
| OCI- | DSMZ | ACC 582 | Germany |
| AML3 | Inhoffenstraße 7B/38124 | | |
| | Braunschweig GERMANY | | |
| MOLM- | DSMZ | ACC 554 | Germany |
| 13 | Inhoffenstraße 7B/38124 | | |
| | Braunschweig GERMANY | | |
| MV- | DSMZ | ACC 508 | Germany |
| 4; 11 | Inhoffenstraße 7B/38124 | | |
| | Braunschweig GERMANY | | |
| HL-60 | DSMZ | ACC 3 | Germany |
| | Inhoffenstraße 7B/38124 | | |
| | Braunschweig GERMANY | | |
| THP-1 | DSMZ | ACC 16 | Germany |
| | Inhoffenstraße 7B/38124 | | |
| | Braunschweig GERMANY | | |
| K562 | LGC Standards S.r.I-ATCC./ | ATCC-CCL- | Italy |
| | Via Carducci, 39 20099 | 243 | |
| | Sesto San Giovanni (MI) Italy | | |
| SHSY5Y | LGC Standards S.r.I-ATCC./ | ATCC-CRL- | Italy |
| | Via Carducci, 39 20099 | 2266 | |
| | Sesto San Giovanni (MI) Italy | | |
| IMR-32 | LGC Standards S.r.I-ATCC./ | ATCC-CCL- | Italy |
| | Via Carducci, 39 20099 | 127 | |
| | Sesto San Giovanni (MI) Italy | | |
| SK-N- | LGC Standards S.r.I-ATCC./ | ATCC-CRL- | Italy |
| BE(2) | Via Carducci, 39 20099 | 2271 | |
| | Sesto San Giovanni (MI) Italy | | |
| SK-N- | LGC Standards S.r.I-ATCC./ | HTB-11 | Italy |
| SH | Via Carducci, 39 20099 | | |
| | Sesto San Giovanni (MI) Italy | | |
| LAN-1 | DSMZ | ACC 655 | Germany |
| | Inhoffenstraße 7B/38124 | | |
| | Braunschweig GERMANY | | |
| SK-ES- | LGC Standards S.r.I-ATCC./ | HTB-86 | Italy |
| 1 | Via Carducci, 39 20099 | | |
| | Sesto San Giovanni (MI) Italy | | |
| A-673 | LGC Standards S.r.I-ATCC./ | CRL-1598 | Italy |
| | Via Carducci, 39 20099 | | |
| | Sesto San Giovanni (MI) Italy | | |
| RD | LGC Standards S.r.I-ATCC./ | ATCC-CCL- | Italy |
| | Via Carducci, 39 20099 | 136 | |
| | Sesto San Giovanni (MI) Italy | | |
| RH30 | DSMZ | ACC 489 | Germany |
| | Inhoffenstraße 7B/38124 | | |
| | Braunschweig GERMANY | | |
| RH41 | DSMZ | ACC 592 | Germany |
| | Inhoffenstraße 7B/38124 | | |
| | Braunschweig GERMANY | | |
| 143B | LGC Standards S.r.I-ATCC./ | ATCC-CRL- | Italy |
| | Via Carducci, 39 20099 | 8303 | |
| | Sesto San Giovanni (MI) Italy | | |
| HOS | LGC Standards S.r.I-ATCC. | ATCC-TCP- | Italy |
| | /Via Carducci, 39 20099 | 1009 | |
| | Sesto San Giovanni (MI) Italy | | |
| U-2OS | LGC Standards S.r.I-ATCC./ | | |
| | Via Carducci, 39 20099 | | |
| | Sesto San Giovanni (MI) Italy | | |
| SAOS- | LGC Standards S.r.I-ATCC./ | | |
| 2 | Via Carducci, 39 20099 | | |
| | Sesto San Giovanni (MI) Italy | | |
| U87 | LGC Standards S.r.I-ATCC./ | HTB-14 | Italy |
| | Via Carducci, 39 20099 | | |
| | Sesto San Giovanni (MI) Italy | | |
| DAOY | LGC Standards S.r.I-ATCC./ | ATCC HTB- | Italy |
| | Via Carducci, 39 20099 | 186 | |
| | Sesto San Giovanni (MI) Italy | | |
| D283 | LGC Standards S.r.I-ATCC./ | HTB-185 | Italy |
| | Via Carducci, 39 20099 | | |
| | Sesto San Giovanni (MI) Italy | | |
| 293 T | LGC Standards S.r.I-ATCC./ | ATCC CRL- | Italy |
| | Via Carducci, 39 20099 | 3216 | |
| | Sesto San Giovanni (MI) Italy | | |
| PANC- | LGC Standards S.r.I-ATCC./ | CRL-1469 | Italy |
| 1 | Via Carducci, 39 20099 | | |
| | Sesto San Giovanni (MI) Italy | | |
| MIAPa | LGC Standards S.r.I-ATCC./ | CRL-1420 | Italy |
| Ca-2 | Via Carducci, 39 20099 | | |
| | Sesto San Giovanni (MI) Italy | | |
| BxPC-3 | LGC Standards S.r.I-ATCC./ | CRL-1687 | Italy |
| | Via Carducci, 39 20099 | | |
| | Sesto San Giovanni (MI) Italy | | |
| CFPAC- | LGC Standards S.r.I-ATCC./ | CRL-1918 | Italy |
| 1 | Via Carducci, 39 20099 | | |
| | Sesto San Giovanni (MI) Italy | | |
| AsPC1 | LGC Standards S.r.I-ATCC./ | CRL-1682 | Italy |
| | Via Carducci, 39 20099 | | |
| | Sesto San Giovanni (MI) Italy | | |
| HT-29 | LGC Standards S.r.I-ATCC./ | HTB-38 | Italy |
| | Via Carducci, 39 20099 | | |
| | Sesto San Giovanni (MI) Italy | | |
| HCT- | LGC Standards S.r.I-ATCC./ | CCL-247 | Italy |
| 116 | Via Carducci, 39 20099 | | |
| | Sesto San Giovanni (MI) Italy | | |
| CaCo-2 | LGC Standards S.r.I-ATCC./ | HTB-37 | Italy |
| | Via Carducci, 39 20099 | | |
| | Sesto San Giovanni (MI) Italy | | |
| SW480 | LGC Standards S.r.I-ATCC./ | CCL-227 | Italy |
| | Via Carducci, 39 20099 | | |
| | Sesto San Giovanni (MI) Italy | | |
| DLD-1 | LGC Standards S.r.I-ATCC./ | CCL-221 | Italy |
| | Via Carducci, 39 20099 | | |
| | Sesto San Giovanni (MI) Italy | | |
| Lovo | LGC Standards S.r.I-ATCC./ | CCL-229 | Italy |
| | Via Carducci, 39 20099 | | |
| | Sesto San Giovanni (MI) Italy | | |
| SK-BR- | LGC Standards S.r.I-ATCC./ | HTB-30 | Italy |
| 3 | Via Carducci, 39 20099 | | |
| | Sesto San Giovanni (MI) Italy | | |

The biological material of human origin (see table 6) used in these experiments has been sampled after that the donors signed a written informed consent, in accordance with rules set by the Institutional Review Board (IRB) of Bambino Gesü Children's Hospital of Rome (OPBG; Approval of Ethical Committee N^{o}969/2015 prot. N^{o}669LB).

TABLE 6

| Biological | | | |
|---|---|---|---|
| material from | | | |
| human body | Collecting place | Authorization protocol | Country |
| PBMC di donatori | OPBG | spontaneous donation | Italy |
| sani | | AEC N969/2015 Protocol | |
| | | N669LB | |
| Cellule T/NK | OPBG | spontaneous donation | Italy |
| | | AEC N969/2015 Protocol | |
| | | N669LB | |

Regarding OGM, the experiments have been authorized by the Italian Ministry of Health (notification RM/IC/Op2/18/007):
Plant authorization (RM/IC/Imp2/18/003);
Authorization for the use of genetically modified microorganisms (RM/IC/Op2/18/006).

### Design of B7-H3.CAR Plasmid (Constructs)

A clinical grade “third” generation of retrovirus bicistronic vector SFG have been designed, allowing the simultaneous expression of two transgenes, namely iC9 suicide gene and the cassette anti-B7-H3 single-chain variable fragment (scFv), derived from a murine antibody of IgG (Ne97) class or IgM (M5B14), linked via a codon optimized human CD8 spacer-transmembrane domain, to the codon optimized signaling costimulatory domain CD28, the codon optimized signaling costimulatory domain 4-1BB (CD137) or OX40 and CD3-ζ.

In particular, the iC9 gene contains the intracellular portion of the human caspase 9 protein, a pro-apoptotic molecule, fused to a drug-binding domain derived from human FK506-binding protein FKBP12. A T2A self-cleaving peptides sequence separate iC9 from CAR sequence.

The scFv Ne97 or M5B14 is cloned in frame with codon optimized CD34 derived epitope of 16 aa (as trackable marker), linked by spacer of 40 aa to bind the codon optimized human CD8-transmembrane domain (CD8aTM) of 23 aa. The signal run from extracellular portion of B7-H3 scFv to intracellular portion of CD3-ζ chain (113aa) through two costimulatory molecules: CD28 endodomain (41aa) and 4-1BB endodomain (42aa) or OX40 endodomain (36 aa).

### Generation of Retroviral Vectors and Transduction Method of T-Cells

Retroviral supernatant was generated in 293T-cells (PMID:32381575, PMID:20686963) and quantified by Retro-X™ qRT-PCR Titration Kit (Takara) to be used at 10⁹ retrovirus-copies/0.5×106 T-cells. The supernatant was used to transduce primary T cells derived from peripheral blood mononuclear cells of healthy donors (Ethical Committee Approval N^{o}969/2015 prot. N^{o}669LB).

In particular, T lymphocytes were activated with immobilized OKT3 (1 μg/ml, e-Bioscience Inc.; San Diego, CA, USA) and anti-CD28 (1 μg/ml, BD Biosciences, Europe) antibodies in the presence of interleukin-2 (IL2) or combination of recombinant human interleukin-7 (IL7, 10 ng/ml; R&D; USA) and recombinant human interleukin-15 (IL-15, 5 ng/ml; R&D). Activated T cells were transduced on day 3 in 24-well plates pre-coated with recombinant human RetroNectin (Takara-Bio. Inc; Japan) using a specific retroviral supernatant and the specific above-described cytokines. At day 5 from transduction, the T cells are expanded in “CTL complete medium” containing 45% RPMI 1640 and 45% Click's medium (Sigma-Aldrich, Co.; Usa) supplemented with 10% FBS and 2 mM Glutamax, and fed twice a week with the specific above described cytokines (PMID: 29872565).

Generation of eGFP-Firefly-Luciferase Cell Lines.

The retroviral vector encoding eGFP-Firefly-Luciferase (eGFP-FFLuc) was used in selected experiments to label B7-H3 positive (B7-H3+) or B7-H3 negative (B7-H3−) tumor cells:

### B7-H3+ Tumor Cell Lines:

Hodgkin's Lymphoma cell line HDLM-2
Non-Hodgkin's Ki-positive Large Cell Lymphoma (Karpas 299)
Acute myeloid leukemia cell line OCI-AML3 and MV4-11
Pre-B Lymphoblastic leukemia 697
Neuroblastoma cell line SHSY5Y and IMR-32
Ewing sarcoma cell lines: A673 and SK-ES-1
Embryonal Rhabdomyosarcoma (ERMS) cell line RD
Alveolar Rhabdomyosarcoma (ARMS) cell lines RH30 and RH41
Medulloblastoma cell lines: D283 and DAOY
Glioblastoma cell lines: U87 and U373
Osteosarcoma (OS) cell lines: 143B; HOS; U2-OS
Pancreatic carcinoma cell line (Mia PaCa-2)
Colon carcinoma cell line (HCT-116)

### B7-H3− (Negative) Tumor Cell Lines:

Hodgkin's Lymphoma cell line L428

### Origin of all Cell Lines Used in the Study.

Hodgkin's Lymphomas (HL) HDML-2 and L428 and the B cell precursor leukemia Ph+BV173, the TOM-1 were obtained from DSMZ. The B cell precursor leukemia 697 and RS4;11 were obtained from DSMZ. Burkitts Lymphoma Daudi was obtained from ATCC. Non-Hodgkin's Lymphoma (NHL) Karpas 299 was obtained from Sigma-Aldrich. The Acute Myeloid Leukemia OCI-AML3, MOLM-13 were obtained from DSMZ. The Acute Myeloid Leukemia MV-4;11, HL-60 and THP-1 were obtained from DSMZ. The myelogenous leukemia cell line K562 was from LGC Standards-ATCC. The neuroblastoma cell lines SHSY5Y, IMR-32, SK-N-BE(2), SK-N-SH were obtained from LGC Standards-ATCC. The GD2-negative subclone of SHSY5Y [SHSY5Y GD2(neg)] cell line has been selected with the BD FACSAria III sorter (PMID: 29872565). The neuroblastoma cell lines LAN-1 was obtained from DSMZ. The Ewing's sarcoma cell lines SK-ES-1 and A-673 were obtained from LGC Standards-ATCC. The ERMS RD was obtained from LGC Standards-ATCC. The ARMS RH30 and RH41 were obtained from DSMZ. The OS cell lines: 143B, HOS, U-20S and SAOS-2 were obtained from LGC Standards-ATCC. The Glioblastoma U87 was obtained from LGC Standards-ATCC. The medulloblastomas DAOY and D283 were obtained from LGC Standards-ATCC. The embryonic kidney 293T cell line were obtained from LGC Standards-ATCC. The pancreatic tumor cells PANC-1, MIAPaCa-2, BxPC-3, CFPAC-1 and AsPC1 were obtained from LGC Standards-ATCC. The colon cancer cells HT-29, HCT-116, CaCo-2, SW480, DLD-1, and Lovo were obtained from LGC Standards-ATCC. The breast cancer SK-BR-3 was obtained from LGC Standards-ATCC.

Cells were maintained in a humidified atmosphere containing 5% CO2 at 37° C. All cell lines were routinely tested for Mycoplasma and for surface expression of target antigens. All cell lines have been authenticated by STR analysis in the certificated lab “BMR Genomics s.r.l.”

### Phenotypic Analysis.

Expression of cell surface molecules was determined by flow cytometry using standard methodology. The following monoclonal antibodies (mAbs) were used: CD3, CD4, CD8, CD45, CD56, CD279 (PD1). The expression of CAR on B7-H3.CART cells was detected using a specific anti-CD34+(QBENd10V Clone). T-cell receptor (TCR)-Vβ repertoire on NT T cells and CAR-T cells was evaluated at day +15 and day +30, using a panel of 24 different TCR Vβ-specific mAbs (IO TEST Beta Mark TCR-Vβ repertoire kit, BC) used in association with CD3 specific mAb (BD Biosciences) and isotype control mAb (BD Biosciences). Samples were analyzed with a BD LSRFortessa X-20. Flow cytometry profiles were analyzed using the FACSDiva software (BD Biosciences). For each sample, a minimum of 20,000 events have been analyzed.

### TCR V Beta (β) Repertoire

To evaluate the relative TCR Vβ repertoire distribution between NT and CAR modified T cells at day +15 the IOTest® Beta Mark Kit (Beckman Coulter) was used. This method uses a multi-parametric analysis tool designed for quantitative determination of the TCR Vβ repertoire of human T lymphocytes by flow cytometry.

### Chromium Release Assay.

The cytotoxic activity was evaluated using a 6-hour ⁵¹Cr release assay as previously described (PMID: 29872565). Target cells were: Hodgkin's Lymphoma cell line HDLM2 (CD276=99.9%), the acute myeloid leukemia cell line OCI-AML3 (CD276=98.7%), the neuroblastoma SHSY5Y (CD276=99.9%) and Hodgkin's Lymphoma cell line L428 (B7H3 Neg). ⁵¹Cr labeled target cells incubated in medium alone or 1% Triton X-100 were used to determine spontaneous and maximal ⁵¹Cr release, respectively. After 6 hours of co-culture between effector and target cells, the supernatant was collected and the radioactivity measured with a gamma counter. The mean percentage of specific lysis of triplicate wells was calculated as follows: [(Experimental release-spontaneous release)/(maximal release-spontaneous release)]×100.

### Co-Culture Assay.

For co-culture experiments, NT and B7-H3.CAR T lymphocytes were plated at 0.2×10⁶ cells/well in 24-well plates at the indicated E:T ratios. Following 6 days of incubation at 37° C., tumor cells and T cells were collected and residual tumor cells and T cells assessed by fluorescence-activated cell-sorting (FACS) analysis based on CD3 expression (Effector T cells) and GFP (tumor cell line).

### Cytokine Profile.

Supernatant from co-culture experiments was collected at 24 hours to measure IFNγ, IL-2, Granzyme B and TNF-α using the Ella Automated Immunoassay System (R&D System, USA).

Quantitative Real-Time PCR (qPCR)

Total DNA was purified by QIAamp DNA Mini Kit (Qiagen) according to the manufacturer's instructions. TaqMan primer/probes were designed specific for the inducible Caspase 9 (iC9) suicide gene. qPCR was performed by using the 7900 HT fast-Real Time-PCR System and ViiA7 system (ThermoFisher Scientific, USA) and TaqMan Gene Expression Master Mix (ThermoFisher Scientific, USA).

### Administration of the Dimerizing Drug AP1903 to Induce the Activation of the Safety Switch iC9

T cells and B7-H3.CAR T-cells were exposed to 10 nM AP1903 (cat #6130, Bio-techne brand) for 48 hours and residual viable cells were stained with Annexin-V/7AAD (BD Pharmingen) and analysed by flow cytometry.

### In Vivo Experiments

To investigate the in vivo antitumor activity of NE97.B7-H3.CAR T-cells on systemic rhabdomyosarcoma model, 0.25×10⁶ RH30-GFP-FF-Luc cells were Intravenous injected (i.v.), in 6-8 week old NOD scid gamma (NSG)mice (NOD.Cg-Prkdcscid Il2rgtm1Wjl/SzJ; from Charles River).

To investigate the in vivo antitumor activity of NE97.B7-H3.CAR T-cells on orthotopic model of brain tumor, NSG mice engrafted with 0.2×10⁶ of MB cell line D283.GFP-FF-luciferase in the brain, by stereotaxic system. After tumor engraftment, the mice received only one i.v. injection of effector T cells (10×10⁶/mouse). This means that no more than one infusion of the effector cells or fractionated dosages are necessary. Tumor growth was evaluated using IVIS imaging system (PerkinElmer, USA), Briefly, a constant region of interest was drawn over the mouse and the intensity of the signal measured, every week, as total photon/sec/cm2/sr (p/s/cm2/sr), as previously described (PMID: 20686963). Mice were maintained in the animal facility at Plaisant Castel Romano (Rome, Italy). All in vivo experiments were in compliance with the ethical international, EU and national requirements and were approved by the Italian Health Ministry (N^{o}88/2016-PR). The circulating human T cells were evaluated periodically in mice peripheral blood.

### Statistical Analysis.

Data are summarized as average±standard deviation (SD). Student t-test (two-sided) was used to determine statistically significant differences between samples, with p value <0.05 indicating a significant difference. The mouse survival data were analyzed using the Kaplan-Meier survival curves, the log-rank test was used to measure differences between groups. No valuable samples were excluded from the analyses. Animals were excluded only in the event of death after tumor implant, but before T-cell infusion. Mice were matched based on the tumor signal for control and treatment groups before infusion of control or gene-modified T cells. To compare the growth of tumors over time, bioluminescence signal intensity was collected in a blind fashion. Bioluminescence signal intensity was log transformed and then compared using a two-sample t-test.

### Results

The transduction efficiency of primary T cells, growth in IL-2, was similar between the two types of third generation (^{III}) B7-H3.CARs constructs (namely NE97.B7-H3.CAR-28.4-1BBζ and NE97.B7-H3.CAR-28.OX40ζ or M5B14.B7-H3.CAR-28.4-1BBζ and M5B14.B7-H3.CAR-28.OX40ζ) (FIG. 2A and FIG. 2B). However, NE97.B7-H3.CAR-28.OX40ζ CAR T-cells showed higher expression of CAR molecules respect to NE97.B7-H3.CAR-28.4-1BBζ (83.85%±2.35% and 72.20%±9.16% respectively, p=0.048). Interestingly, NE97.B7-H3.CAR-28.OX40ζ CAR T-cells showed more CAR molecules surface expression, as indicated by significant higher Median Fluorescence Intensity (MFI) (20702.25±11047.47) respect to the other three CARs: namely NE97.B7-H3.CAR-28.4-1BBζ (12080.75±9306.41), M5B14.B7-H3.CAR-28.4-1BBζ (3751.00±1856.51) and M5B14.B7-H3.CAR-28.OX40ζ (5253.33±3158.63) (FIG. 2C) (p=0.003, p=0.027 and p=0.014, respectively). This means that NE97.B7-H3.CAR-28.OX40ζ CAR T-cells are better armed to recognize and kill cancer cells.

B7-H3.CAR molecules did not induce any significant proliferative change in genetically modified T cells as compared to non-transduced control (NT) T cells and was superimposable during the first two weeks of culture (FIG. 3A), and did not induce any TCR Vβ family selection (FIG. 3B).

Independently of the co-stimulatory combination used in the B7-H3.CARs construct, no significant difference was observed in the suicide gene inducible caspase 9 (iCasp9) in vitro activity (FIG. 3C) and the Vector copy Number of transduced T cells was below 12 (FIG. 3D).

The expression of B7-H3 (CD276, B7H3, B7RP-2) antigen was evaluated, by flow cytometry, on several haematological tumor cell lines. Specifically, B7-H3 is expressed in two out of 4 lymphoma lines (both Hodgkin lymphomas (HL) and non-Hodgkin lymphomas (NHL) (FIG. 4A), in one out of 4 B-acute lymphoblastic leukemia cell line (FIG. 4B), in four out of five acute myeloid leukemia cell lines (FIG. 4C) and in one Chronic myeloid leukaemia (CML) (FIG. 4D).

In solid tumours, high expression of B7-H3 was found in all neuroblastoma (NB) cell lines tested (FIG. 4E), all sarcoma cell lines tested (FIG. 4F), all brain tumours tested (FIG. 4G), pancreatic cancers (FIG. 4H), colon cancers (FIG. 4I) and adenocarcinoma breast cancer (FIG. 4J).

To compare the cytolytic activity of ^{III}B7-H3.CAR T cells including either of the two scFv and the two different costimulatory signalling domains, a standard 6-hr ⁵¹Cr release assay was performed. FIG. 5 (A-D) shows that both scFv ^{III}B7-H3.CAR T cells (NE97 and M5B14), expressing either CD28.4-1BB or CD28.OX40 selectively kill with the same efficiency B7-H3+ Hodgkin Lymphomas HDLM-2 (FIG. 5A), but not the B7-H3 negative Hodgkin Lymphomas L428 (FIG. 5B). They also kill with the same efficiency B7-H3+ Acute Myeloid Leukemia OCI-AML3 (FIG. 5C).

Although all four ^{III}B7-H3.CAR T cells kill, with similar efficiency, the neuroblastoma (NB) tumor cell line SHSY5Y, at a very low E:T ratio (5:1), NE97.B7-H3.CAR-28.OX40ζ CAR T-cells kill the NB cell line with significant higher efficiency (42.5%±7.38%) respect to M5B14.B7-H3.CAR-28.4-1BBζ (29.48%±12.33%, p=0.028) or M5B14.B7-H3.CAR-28.OX40ζ T cells (30.43%±7.37%, p=0.019) (FIG. 5D). This assay evaluates the ability of the effector cell to recognize and kill cancer cells after a few hours of contact (6 hours). Although they are all equally active in the ratios 40:1, 20:1 and 10:1; however, when NE97.B7-H3.CAR-28.OX40ζ CAR T-cells were co-cultured at the lower E:T ratio (5:1), kill higher percentage of cancer cells (42.5%), therefore they are more efficient than the others constructs.

In long term co-culture (6 days of co-culture), at an E:T ratio of 1:1, ^{III}NE97.B7-H3.CAR T cells (expressing either CD28.4-1BB or CD28.OX40) significantly control, with higher efficiency, the tumor growth of NB tumor cell line SHSY5Y (FIG. 6A) and IMR-32 (FIG. 6B). The superior activity of ^{III}NE97.B7-H3.CAR T cells respect to M5B14.B7-H3.CAR T cells, with CD28.4-1BB or CD28.OX40 as costimulatory domains, was confirmed also against the Ewing sarcoma cell line A673 (FIG. 6C) and the embryonal rhabdomyosarcoma (ERMS) cell line RD (FIG. 6D).

Interesting to note that, all four ^{III}B7-H3.CAR T cells kill with the same efficiency several lymphoma and leukaemia cell lines as: the Hodgkin Lymphomas cell lines: HDML-2 (FIG. 6E), the acute myeloid leukemia (AML FAB M4) cell line OCI-AML3 (FIG. 6F), the acute monoblastic/monocytic leukemia cell line MV4-11 (FIG. 6G) and the B cell precursor leukemia cell line 697 (FIG. 6H).

In brain tumor cell lines, although all four ^{III}B7-H3.CAR T cells (grown in IL-2) kill with the same efficiency the MB cell line D283 (FIG. 7A), ^{III}B7-H3.CAR T cells, carrying the CD28.OX40 as costimulatory domains, exert a significant higher tumor control against desmoplastic cerebellar MB cell line DAOY as target: effector/target ratio of 1:1 (9.68%±11.10% and 19.03%±9.89% residual tumor cell after co-culture with NE97.B7-H3.CAR-28.OX40ζ T cells and M5B14.B7-H3.CAR-28.OX40ζ T cells, respectively) as compared to control NT T cells (36.10%±15.95%, in both case p<0.05) (FIG. 7B). To notice there are no significant differences in tumor control (DAOY) between the two CARs when both B7-H3.CAR T-cells (NE97 and M5B14) carrying CD28.OX40, as costimulatory domains (p=0.089, not significant). In fact, table 8 below shows that the two CARs with CD28.OX40 control most tumors with the same efficiency (p=ns).

In Glioblastoma cell lines U87 (FIG. 70) and U373 (FIG. 7D) all four ^{III}B7-H3.CAR T cells exert a similar significant tumor control, as compared to control NT T cells.

In table 7-9, it was summarized the long-term co-culture results (as % of residual tumor cells) collected for all ^{III}B7-H3.CAR T cells co-cultures with solid tumours: NB and sarcoma cell lines (table 7); brain tumours: glioblastoma and MB cell lines (table 8); haematological tumours: lymphoma, AML and ALL leukaemia cell lines (table 9).

Table 7 summarizes the long-term co-culture results (as % of residual tumor cells) collected for all ^{III}B7-H3.CAR T cells co-cultures with solid tumours as neuroblastoma (NB) and sarcoma cell lines. T test value are also reported.

TABLE 7

| | | | Effector Cells | | |
|---|---|---|---|---|---|
| | | | NT-T Cells | | |
| Co-culture assay E:T 1:1 | | | (% tumor) | | |
| Tumor: NB (SHSY5Y) | | | 64.6% ± 10.1% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.0002 | (% tumor) | vs NT | 0.0001 |
| 9.3% ± 13.2% | vs OPBG- | ns | 1.0% ± 1.1% | vs OPBG- | ns |
| | 158 | | | 157 | |
| | vs OPBG- | 0.008 | | vs OPBG- | 0.014 |
| | 159 | | | 159 | |
| | vs OPBG- | ns | | vs OPBG- | ns |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | ns | (% tumor) | vs NT | ns |
| 55.6% ± 29.1% | vs OPBG- | 0.008 | 35.5% ± 29.0% | vs OPBG- | ns |
| | 157 | | | 157 | |
| | vs OPBG- | 0.014 | | vs OPBG- | ns |
| | 158 | | | 158 | |
| | vs OPBG- | 0.032 | | vs OPBG- | 0.032 |
| | 160 | | | 159 | |
| | | | Effector Cells | | |
| | | | NT-T Cells | | |
| Co-culture assay E:T 1:1 | | | (% tumor) | | |
| Tumor: NB (HDML-2) | | | 85.7% ± 6.4% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.001 | (% tumor) | vs NT | 0.0001 |
| 9.7% ± 12.0% | vs OPBG- | ns | 1.4% ± 1.6% | vs OPBG- | ns |
| | 158 | | | 157 | |
| | vs OPBG- | ns | | vs OPBG- | 0.003 |
| | 159 | | | 159 | |
| | vs OPBG- | ns | | vs OPBG- | ns |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.002 | (% tumor) | vs NT | 0.002 |
| 5.7% ± 1.8% | vs OPBG- | ns | 2.5% ± 2.7% | vs OPBG- | ns |
| | 157 | | | 157 | |
| | vs OPBG- | 0.0031 | | vs OPBG- | ns |
| | 158 | | | 158 | |
| | vs OPBG- | 0.021 | | vs OPBG- | 0.021 |
| | 160 | | | 159 | |
| | | | Effector Cells | | |
| | | | NT-T Cells | | |
| Co-culture assay E:T 1:1 | | | (% tumor) | | |
| Tumor: Ewing sarcoma (A673) | | | 75.6% ± 12.6% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.002 | (% tumor) | vs NT | 0.001 |
| 2.7% ± 2.3% | vs OPBG- | ns | 0.9% ± 0.8% | vs OPBG- | ns |
| | 158 | | | 157 | |
| | vs OPBG- | 0.00004 | | vs OPBG- | 0.00003 |
| | 159 | | | 159 | |
| | vs OPBG- | ns | | vs OPBG- | 0.048 |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | ns | (% tumor) | vs NT | 0.008 |
| 55.7% ± 7.6% | vs OPBG- | 0.00004 | 24.9% ± 19.1% | vs OPBG- | ns |
| | 157 | | | 157 | |
| | vs OPBG- | 0.00003 | | vs OPBG- | 0.048 |
| | 158 | | | 158 | |
| | vs OPBG- | 0.043 | | vs OPBG- | 0.043 |
| | 160 | | | 159 | |
| | | | Effector Cells | | |
| | | | NT-T Cells | | |
| Co-culture assay E:T 1:1 | | | (% tumor) | | |
| Tumor: Rhabdomyosacoma (RD) | | | 63.9% ± 15.7 | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.004 | (% tumor) | vs NT | 0.004 |
| 2.28% ± 1.9% | vs OPBG- | ns | 1.4% ± 0.9% | vs OPBG- | ns |
| | 158 | | | 157 | |
| | vs OPBG- | 0.0013 | | vs OPBG- | 0.0009 |
| | 159 | | | 159 | |
| | vs OPBG- | ns | | vs OPBG- | 0.0042 |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.013 | (% tumor) | vs NT | 0.001 |
| 26.70% ± 7.4% | vs OPBG- | 0.0013 | 5.1% ± 1.1% | vs OPBG- | ns |
| | 157 | | | 157 | |
| | vs OPBG- | 0.0009 | | vs OPBG- | 0.004 |
| | 158 | | | 158 | |
| | vs OPBG- | 0.048 | | vs OPBG- | 0.048 |
| | 160 | | | 159 | |

Table 8 summarizes the long-term co-culture results (as % of residual tumor cells) collected for all ^{III}17-H3.CAR T cells co-cultures with brain tumours as Glioblastoma and medulloblastoma cell lines. T test value are also reported.

TABLE 8

| | | | Effector Cells | | |
|---|---|---|---|---|---|
| | | | NT-T Cells | | |
| Co-culture assay E:T 1:1 | | | (% tumor) | | |
| Tumor: Glioblastoma (U87) | | | 53.4% ± 7.2% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.015 | (% tumor) | vs NT | 0.001 |
| 11.9% ± 9.1% | vs OPBG- | ns | 3.3% ± 2.0% | vs OPBG- | ns |
| | 158 | | | 157 | |
| | vs OPBG- | ns | | vs OPBG- | 0.017 |
| | 159 | | | 159 | |
| | vs OPBG- | ns | | vs OPBG- | ns |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | ns | (% tumor) | vs NT | 0.005 |
| 32.40% ± 14% | vs | ns | 10.4% ± 9.0% | vs OPBG- | ns |
| | OPBG- | | | 157 | |
| | 157 | | | | |
| | vs | 0.017 | | vs OPBG- | 0.017 |
| | OPBG- | | | 158 | |
| | 158 | | | | |
| | vs | ns | | vs OPBG- | ns |
| | OPBG- | | | 159 | |
| | 160 | | | | |
| | | | Effector Cells | | |
| Co-culture assay E:T 1:1 | | | NT-T Cells | | |
| Tumor: Glioblastoma | | | (% tumor) | | |
| astrocytoma (U373) | | | 61.4% ± 18.2% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.020 | (% tumor) | vS NT | 0.014 |
| 12.4% ± 14.4% | vs | ns | 5.2% ± 6.3% | vs OPBG- | ns |
| | OPBG- | | | 157 | |
| | 158 | | | | |
| | vs | ns | | vs OPBG- | ns |
| | OPBG- | | | 159 | |
| | 159 | | | | |
| | vs | ns | | vs OPBG- | ns |
| | OPBG- | | | 160 | |
| | 160 | | | | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.043 | (% tumor) | vs NT | 0.019 |
| 15.6% ± 14.6% | vs | ns | 6.3% ± 8.2% | vs OPBG- | ns |
| | OPBG- | | | 157 | |
| | 157 | | | | |
| | vs | ns | | vs OPBG- | ns |
| | OPBG- | | | 158 | |
| | 158 | | | | |
| | vs | ns | | vs OPBG- | ns |
| | OPBG- | | | 159 | |
| | 160 | | | | |
| | | | Effector Cells | | |
| Co-culture assay E:T 1:1 | | | NT-T Cells | | |
| Tumor: Medulloblastoma | | | (% tumor) | | |
| (D283) | | | 74.5% ± 7.1% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.003 | (% tumor) | vs NT | 0.003 |
| 2.6% ± 1.6% | vs | ns | 0.9% ± 0.5% | vs OPBG- | ns |
| | OPBG- | | | 157 | |
| | 158 | | | | |
| | vs | ns | | vs OPBG- | ns |
| | OPBG- | | | 159 | |
| | 159 | | | | |
| | vs | ns | | vs OPBG- | ns |
| | OPBG- | | | 160 | |
| | 160 | | | | |
| Effector Cells | | | Effector Cells | | |
| M5B14.B7-H3.CAR-28.4-1BB | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.004 | (% tumor) | vs NT | 0.002 |
| 2.8% ± 1.2% | vs | ns | 1.8% ± 1.0% | vs OPBG- | ns |
| | OPBG- | | | 157 | |
| | 157 | | | | |
| | vs | ns | | vs OPBG- | ns |
| | OPBG- | | | 158 | |
| | 158 | | | | |
| | vs | ns | | vs OPBG- | ns |
| | OPBG- | | | 159 | |
| | 160 | | | | |
| Co-culture assay E:T 1:1 | | | Effector Cells | | |
| Tumor: Desmoplastic | | | NT-T Cells | | |
| cerebellar medulloblastoma | | | (% tumor) | | |
| (DAOY) | | | 36.1% ± 15.9% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | ns | (% tumor) | vs NT | 0.034 |
| 27.1% ± 17.3% | vs | ns | 9.7% ± 11.1% | vs OPBG- | ns |
| | OPBG- | | | 157 | |
| | 158 | | | | |
| | vs | ns | | vs OPBG- | ns |
| | OPBG- | | | 159 | |
| | 159 | | | | |
| | vs | ns | | vs OPBG- | ns |
| | OPBG- | | | 160 | |
| | 160 | | | | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | ns | (% tumor) | vs NT | 0.047 |
| 32.2% ± 18.4% | vs | ns | 19.0% ± 9.9% | vs OPBG- | ns |
| | OPBG- | | | 157 | |
| | 157 | | | | |
| | vs OPBG- | ns | | vs OPBG- | ns |
| | 158 | | | 158 | |
| | vs | ns | | vs OPBG- | ns |
| | OPBG- | | | 159 | |
| | 160 | | | | |

Table 9 summarizes the long-term co-culture results (as % of residual tumor cells) collected for all B7-H3.CAR T cells co-cultures with lymphoma tumor cell, acute myeloid leukaemia, acute monoblastic/monocytic leukaemia and lymphoblastic leukaemia cell lines. T test value are also reported.

TABLE 9

| | | | Effector Cells | | |
|---|---|---|---|---|---|
| Co-culture assay E:T 1:1 | | | NT-T Cells | | |
| Tumor: Hodgkin Lymphoma | | | (% tumor) | | |
| (HDML-2) | | | 85.7% ± 6.4% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.001 | (% tumor) | vs NT | 0.001 |
| 9.7% ± 12.0% | vs | ns | 1.4% ± 1.6% | vs OPBG- | ns |
| | OPBG- | | | 157 | |
| | 158 | | | | |
| | vs | ns | | vs OPBG- | 0.003 |
| | OPBG- | | | 159 | |
| | 159 | | | | |
| | vs | ns | | vs OPBG- | ns |
| | OPBG- | | | 160 | |
| | 160 | | | | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.002 | (% tumor) | vs NT | 0.002 |
| 5.7% ± 1.8% | vs | Ns | 2.5% ± 2.7% | vs OPBG- | ns |
| | OPBG- | | | 157 | |
| | 157 | | | | |
| | vs | 0.003 | | vs OPBG- | ns |
| | OPBG- | | | 158 | |
| | 158 | | | | |
| | vs | 0.021 | | vs OPBG- | 0.021 |
| | OPBG- | | | 159 | |
| | 160 | | | | |
| | | | Effector Cells | | |
| Co-culture assay E:T 1:1 | | | NT-T Cells | | |
| Tumor: Acute myeloid leukemia | | | (% tumor) | | |
| (OCI-AML-3) | | | 70.6% ± 20.5% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vs NT | 0.004 | (% tumor) | vs NT | 0.005 |
| 4.6% ± 8.7% | vs | ns | 2.5% ± 4.6% | vs OPBG- | ns |
| | OPBG- | | | 157 | |
| | 158 | | | | |
| | vs | ns | | vs OPBG- | ns |
| | OPBG- | | | 159 | |
| | 159 | | | | |
| | vs | ns | | vs OPBG- | ns |
| | OPBG- | | | 160 | |
| | 160 | | | | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vsNT | 0.001 | (% tumor) | vs NT | 0.002 |
| 2.9% ± 5.2% | vs | ns | 2.2% ± 3.9% | vs OPBG- | ns |
| | OPBG- | | | 157 | |
| | 157 | | | | |
| | vs | ns | | vs OPBG- | ns |
| | OPBG- 158 | | | 158 | |
| | vs | ns | | vs OPBG- | ns |
| | OPBG- | | | 159 | |
| | 160 | | | | |
| Co-culture assay E:T 1:1 | | | Effector Cells | | |
| Tumor: Acute | | | NT-T Cells | | |
| monoblastic/monocytic | | | (% tumor) | | |
| leukemia (MV4-11 | | | 79.4% ± 1.9% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vS NT | 0.0002 | (% tumor) | vs NT | 0.002 |
| 0.03% ± 0.1% | vs | ns | 0.2% ± 0.1% | vs | ns |
| | OPBG- | | | OPBG- | |
| | 158 | | | 157 | |
| | vs | ns | | vs | ns |
| | OPBG- | | | OPBG- | |
| | 159 | | | 159 | |
| | vs | ns | | vs | ns |
| | OPBG- | | | OPBG- | |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.002 | (% tumor) | vs NT | 0.002 |
| 0.1% ± 0.1% | vs | ns | 0.1% ± 0.01% | vs | ns |
| | OPBG- | | | OPBG- | |
| | 157 | | | 157 | |
| | vs | ns | | vs | ns |
| | OPBG- | | | OPBG- | |
| | 158 | | | 158 | |
| | vs | ns | | vs | ns |
| | OPBG- | | | OPBG- | |
| | 160 | | | 159 | |
| | | | Effector Cells | | |
| Co-culture assay E:T 1:1 | | | NT-T Cells | | |
| Tumor: Pre-B Lymphoblastic | | | (% tumor) | | |
| leukemia (697) | | | 84.0% ± 7.3% | | |
| Effector Cells | | | Effector Cells | | |
| NE97.B7-H3.CAR-28.4-1BBζ | | | NE97.B7-H3.CAR-28.OX40ζ | | |
| (OPBG-157) T-cells | | | (OPBG-158) T-cells | | |
| | t test | | | t test | |
| (% tumor) | vS NT | 0.003 | (% tumor) | vs NT | 0.005 |
| 2.6% ± 0.2% | vs | ns | 4.7% ± 2.8% | vs | ns |
| | OPBG- | | | OPBG- | |
| | 158 | | | 157 | |
| | vs | ns | | vs | ns |
| | OPBG- | | | OPBG- | |
| | 159 | | | 159 | |
| | vs | ns | | vs | ns |
| | OPBG- | | | OPBG- | |
| | 160 | | | 160 | |
| Effector Cells | | | Effector Cells | | |
| M5B14.B7-H3.CAR-28.4-1BBζ | | | M5B14.B7-H3.CAR-28.4-1BBζ | | |
| (OPBG-159) T-cells | | | (OPBG-160) T-cells | | |
| | t test | | | t test | |
| (% tumor) | vs NT | 0.008 | (% tumor) | vs NT | 0.002 |
| 4.4% ± 5.2% | vs | ns | 2.9% ± 1.3% | vs | ns |
| | OPBG- | | | OPBG- | |
| | 157 | | | 157 | |
| | vs | ns | | vs | ns |
| | OPBG- | | | OPBG- | |
| | 158 | | | 158 | |
| | vs | ns | | vs | ns |
| | OPBG- | | | OPBG- | |
| | 160 | | | 159 | |

Although all four ^{III}B7-H3.CAR T cells kill with the same efficiency the haematological tumours as lymphoma (FIG. 6E and leukaemia cell lines (FIG. 6F-H and table 10), for several solid tumor, only ^{III}NE97.B7-H3.CAR T cells eradicate solid tumor cell lines when co-cultured at low 1:1 effector/target (E/T) ratio (FIG. 6A, 6B, 6C and 7C and table 8-9). So, for solid tumours, the experiments were on this CAR model (^{III}NE97.B7-H3.CAR) carrying the CD28.4-1BB or CD28.OX40 as costimulatory domains.

The presence of IL7/lL15 in the culture conditions did not change significantly the fold expansion of CAR-T cells in comparison to conditions comprising IL2 (FIG. 8A).

It was also observed that, when ^{III}NE97.B7-H3.CAR T-cells are prepared in conditions comprising IL7 and IL15, the expression of CAR observed on T cells (FIG. 8 B) was comparable to CAR T expanded in IL2 (FIG. 2 B). Moreover, as observed for the CAR-T cells growth in IL2, NE97.B7-H3.CAR-28.OX40ζ CAR T-cells (growth in IL7/IL15) showed higher expression of CAR molecules respect to NE97.B7-H3.CAR-28.4-1BBζ CAR T-cells (82.32%±7.05% and 72.63%±8.17% respectively, p=0.018).

However, the switching from IL2 to the cocktail IL7/IL15 improves significantly the potency of both ^{III}NE97.B7-H3.CAR T-cells.

In fact, in in vitro long-term co-cultures (6 days of co-culture), at an E:T ratio of 1:1, proved evidences that, ^{III}NE97.B7-H3.CAR T-cells kill more efficiently the desmoplastic cerebellar MB cell line DAOY (FIG. 9A). Interesting the switching from IL-2 to the cocktail IL-7/IL-15 improve significantly the potency of NE97.B7-H3.CAR-28.4-1BBζ CAR T-cells (from 27.1%±17.3% to 0.03%±0.05%, p=0.020) and NE97.B7-H3.CAR-28.OX40ζ CAR T-cells (from 9.7%±11.1% to 0.3%±0.6%, p=0.143).

Both (IL7/IL15) ^{III}NE97.B7-H3.CAR T-cells kill very efficiently all paediatric tumor cell lines analyzed as neuroblastoma cell line: SHSY5Y (FIG. 9B); the osteosarcoma (OS) cell lines: 143B (FIG. 9C), HOS (FIG. 9D) and U2-OS (FIG. 9E); the Alveolar Rhabdomyosarcoma (ARMS) cell lines RH30 (FIG. 9F) and RH41 (FIG. 9G); the ERMS cell line RD (FIG. 9H); the Ewing's Sarcoma A673 (FIG. 9I) and SK-ES-1 (FIG. 9J); the Non-Hodgkin's Ki-positive Large Cell Lymphoma (Karpas 299) (FIG. 9K).

Both (IL-7/IL-15) ^{III}NE97.B7-H3.CAR T-cells kill very efficiently also several adult solid tumor cell lines such as pancreatic cancers: the ductal adenocarcinoma cell line MIA PACA-2 (FIG. 10A), and the colon carcinoma cell line HCT-116 (FIG. 10B).

To compare the real power of the lytic potency of the two ^{III}NE97.B7-H3.CAR T-cells (growth in IL-7/IL-15), the E/T was stressed from ratio (R) 1:1 to 1:32. For DAOY MB cell line, no significant difference in cytolytic activity between CD28.OX40.ζ (IL-7/IL-15) and CD28.4.1BB.ζ (IL-7/IL-15) was observed (FIG. 11A). Noteworthy, the activity of ^{III}NE97.B7-H3.CAR T-cells at low effector/target ratios (R) showed a significant improvement of the in vitro tumor control of 28.OX40.ζ (IL7/lL15) T cells for R1:16 respect to NT T-cells (28.75±31.41% and 67.80%±19.75%, p=0.010) (FIG. 11A).

In ARMS in vitro model, both ^{III}NE97.B7-H3.CAR T-cells produce specific and equal level of a serine protease Granzyme B (GRANB), when co-cultured 24 hours with the tumor cell line RH30 (5216.44 pg/ml±2938.83 pg/ml for CD28.4.1BB.ζ and 5495.67 pg/ml±3343.18 pg/ml for CD28.OX40ζ respectively, p=0.642) (FIG. 12A).

However, when NE97.B7-H3.CAR-28.OX40ζ CAR T-cells are co-cultured 24 h with RH30 cell line produce a significant higher level of Interferon gamma (IFNγ) (704.7 pg/ml±300.2 pg/ml) (FIG. 12B), tumor necrosis factor alpha (TNFα) (516.0 pg/ml±253.5 pg/ml) (FIG. 12C) and IL2 (1389.0 pg/ml±1169.6 pg/ml) (FIG. 12D) respect to NE97.B7-H3.CAR-28.4-1BBζ CAR T-cells (IFNγ=242.7 pg/ml±158.8 pg/ml, p=0.0008; TNFα=151.1 pg/ml±60.44 pg/ml, p=0.0014 and IL2=123.2 pg/ml±154.3 pg/ml, p=0.008, respectively). Similar results have been obtained by using the Ewing's sarcoma cell line A673, (FIG. 12E-H).

Based on endogenous IL2 production, it has been hypothesized that ^{III}NE97.B7-H3.CAR T-cells proliferate significantly more than control NT T-cells, when co-culture with B7-H3+ tumor cell line as RH30 cell line or in the presence of B7-H3 ligand. To verify this hypothesis, a proliferation assay was performed based on the incorporation assay of 3H-thymidine, which is incorporated into new strands of chromosomal DNA during mitotic cell division. As shown in the FIG. 13A, both NT T-cells and CAR T-cells proliferate in the presence of IL-7/IL-15 cytokines; but only ^{III}NE97.B7-H3.CAR T-cells significantly uptake the 3H-thymidine when co-culture with irradiated RH30 cell lines or the B7-H3 ligand.

Based on an in vitro experiment, both ^{III}NE97.B7-H3.CAR T-cells were selected for the next step, e.g. the in vivo experimental evaluation in xenograft mouse model.

Whether the choice of the costimulatory combination of cytokines used during in vitro expansion might influence the in vivo activity of ^{III}NE97.B7-H3.CAR T cells against the pediatric ARMS RH30 (FIG. 14A) was then assessed. The bioluminescence in ARMS-tumor-bearing mice, treated with NT T-cells, rapidly increased up to three hundred times in less than 35 days (8.9e9±5.1e9 p/sec/cm2/sr) (FIGS. 14B and 14C) and mice either died or were sacrificed due to morbidity. The bioluminescence of the tumor in mice treated with ^{III}NE97.B7-H3.CAR T cells was significantly lower at day +35: 1.8e9±1.2e9 p/sec/cm2/sr for 28.4-1BBζ and 1.2e9±1.4e9 p/sec/cm2/sr for 28.OX40ζ, (p=0.035 and p=0.014, respectively).

ARMS-tumor-bearing mice treated with ^{III}NE97.B7-H3.CAR T-cells, independently from costimulatory domains, showed average survival longer (undefined days for mice treated with either NE97.B7-H3.CAR-28.4-1BBζ or NE97.B7-H3.CAR-28.OX40ζ T-cells) compared with mice treated with NT T-cells (FIG. 14D, median survival equal to 49 days).

In brain MB model, NSG mice were engrafted with D283.GFP-FF-luciferase tumor cells in the brain, by stereotaxic system. After tumor engraftment, mice were treated i.v. with effector NT or ^{III}NE97.B7-H3.CAR T-cells (FIG. 15A).

^{III}NE97.B7-H3.CAR T-cells showed a superior anti-tumor activity compared to NT T-cells, which resulted in a significant and rapid reduction of tumor bioluminescence after 30 days from the treatment: 2.8e9±4.7e8 p/sec/cm2/sr for mice treated with NT T-cells vs 6.5e8±6.9e8 p/sec/cm2/sr for mice treated with NE97.B7-H3.CAR-28.4-1BBζ CAR T-cells (p=0.019) and 1.8e5±2.5e4 p/sec/cm2/sr for mice treated with NE97.B7-H3.CAR-28.OX40ζ T-cells (p=0.0002) (FIGS. 15B and 15C). The median overall survival for MB-tumor-bearing mice treated with NE97.B7-H3.CAR-28.OX40ζ T-cells was significantly longer (Undefined) compared to mice treated with NE97.B7-H3.CAR-28.4-1BBζ CAR T-cells (58 days, p=0.0043) or compared to mice treated with NT T-cells (46 days, p=0.0067) (FIG. 15D).

Blood circulating human CD3+T-cells were detected in all MB-tumor-bearing mice treated with NE97.B7-H3.CAR T-cells (FIG. 15E).

Moreover, in MB-tumor-bearing mice treated with NE97.B7-H3.CAR T-cells, CAR+ expression (FIG. 15F) remains stably high, in both NE97.B7-H3.CAR T-cells, from day 14 (81.75%±3.89% for NE97.B7-H3.CAR-28.4-1BBζ CAR T-cells and 85.68%±8.01% for NE97.B7-H3.CAR-28.OX40ζ CAR T-cells, respectively) up to day 45 (83.67%±15.48% for NE97.B7-H3.CAR-28.4-1BBζ CAR T-cells and 80.65%±4.65% for NE97.B7-H3.CAR-28.OX40ζ CAR T-cells, respectively).

Globally all four different clinical grade third generation of B7-H3.CAR T-cells, in particular NE97.B7-H3.CARs, are very active against B7-H3+ leukemias/lymphomas tumor cell lines. However, NE97.B7-H3.CAR T-cells showed a superior anti-tumor activity against solid tumors, compared to NT T-cells and M5B14.B7-H3.CAR T-cells.

All these results make it highly plausible that the constructs according to the present invention can be used to treat efficiently patients affected by given hematological and non-hematological malignances.

### EXAMPLE 2: IN VIVO STUDY CONCERNING THE CYTOLYTIC FUNCTION OF ^{III}NE97.B7-H3.CAR T CELLS IN ACUTE MYELOID LEUKEMIA MV-4-11, CHARACTERIZED BY TRANSLOCATION 4; 11

In AML model, NSG mice were systemically engrafted with B-myelomonocytic leukemia MV-4-11.GFP-FF-luciferase cells (2e6 cells), a tumor cell line with a 4:11 translocation and FLT3 internal tandem duplication. After tumor engraftment, mice were treated i.v. with NT or ^{III}NE97.B7-H3.CAR T-cells (10e6 cells) (FIG. 16A).

^{III}NE97.B7-H3.CAR T-cells showed a superior anti-tumor activity compared to NT T-cells, which resulted in a significant tumor control after 21 days from the treatment (3.03e9 p/sec/cm²/sr for mice treated with NT T-cells vs 1.16e8 p/sec/cm²/sr for mice treated with NE97.B7-H3.CAR-28.4-1BBζ CAR T-cells (p=0.034), and 1.32e7 p/sec/cm²/sr for mice treated with NE97.B7-H3.CAR-28.OX40ζ T-cells (p=0.029). At day +42 a rapid reduction of tumor bioluminescence was observed for five out of five mice treated with NE97.B7-H3.CAR-28.OX40ζ T-cells (p<0.001), and three out of five mice treated with NE97.B7-H3.CAR-28.4-1BBζ CAR T-cells (p<0.001) (FIG. 16B-C). Importantly, the treatment with ^{III}NE97.B7-H3.CAR T-cells did not show in mice any evidence of toxicity including Graft versus Host Disease (GvHD) as demonstrated by increase overtime of body weight of mice (FIG. 16D). However, mice treated with NT T cells showed evident signs of suffering (weight loss) linked to the advancement of the disease (FIG. 16D). The median overall survival (OS) analysis at 90 days in these mice revealed that both the CAR constructs (NE97.B7-H3.CAR-28.OX40ζ vs NE97.B7-H3.CAR-28.4-1BBζ) were able to induce a significant improve of the survival (undefined for both constructs) (p=0.0026 and p=0.0043, respectively) compared to NT T cells (35 days). No difference was detected between the two CAR constructs in term of median OS. At 90 days after T cells infusion 0/5 mice were alive in the NT treated group, 4/5 mice in NE97.B7-H3.CAR-28.4-1BBζ group and 5/5 mice in NE97.B7-H3.CAR-28.OX40ζ group (FIG. 16E).

In order to prove the effect and functionality of our CAR constructs, circulating ^{III}NE97.B7-H3.CAR T-cells were examined in the peripheral blood of mice. As reported in FIG. 17 A-B, CAR T cells were detected in both CAR treated group already at day 13 after T cell treatment and until day 55. Both the CD4 and CD8 CAR T cells demonstrated to be able to expand in vivo with an average expansion peak on day +27. A significant difference in CAR T cell expansion between NE97.B7-H3.CAR-28.4-1BBζ and NE97.B7-H3.CAR-28.OX40ζ was detected in both CD4 (A, p=0,034) and CD8 (B, p=0,005) subsets at day 13. Interesting, it was observed that at day +41 both CD4 and CD8 NE97.B7-H3.CAR-28.4-1BBζ T cells showed a marked reduction in expansion. Furthermore, in both groups of mice treated with CAR T cells, CD4 and CD8 CAR T cells showed functional maturation after tumor engagement. A significant difference was detected in the naïve CD4 subpopulation between NE97.B7-H3.CAR-28.4-1BBζ and NE97.B7-H3.CAR-28.OX40ζ at day 27 and 55 (FIG. 17 C), as well as in the naïve (day 41) and effector memory (day 27) CD8 subpopulation (FIG. 17 D).

1) Anti-B7-H3 chimeric antigen receptor comprising or consisting of, from the N-terminus to the C-terminus:
a) a signal peptide,
b) an anti B7-H3 single chain antibody domain,
c) a hinge,
d) a trans membrane domain,
e) a co-stimulatory signaling domain, and
f) CD3Zeta chain sequence,
wherein said anti B7-H3 single chain antibody domain comprises of anti B7-H3 NE97 hybridoma VL sequence and anti B7-H3 NE97 hybridoma VH sequence linked each other by a linker, and wherein
anti B7-H3 NE97 hybridoma VL sequence comprises CDR1 sequence EIIYSY (SEQ ID NO:1), CDR2 sequence NAK and CDR3 sequence QHHYGTPPYT (SEQ ID NO:2), whereas
anti B7-H3 NE97 hybridoma VH sequence comprises CDR1 sequence GFTFSSYG (SEQ ID NO:3), CDR2 sequence INSGGSYI (SEQ ID NO:4) and CDR3 sequence ARHEGLPLDY (SEQ ID NO:5).

2) Anti-B7-H3 chimeric antigen receptor according to claim 1, wherein
anti B7-H3 NE97 hybridoma VL sequence comprises or consists of
(SEQ ID NO: 6)
DIQMTQSPASLSASVGETVTITCRASEIIYSYLAWYQQKQGKSPQLLVYN
AKTLVEGVPSRFSGSGSGTQFSLKINSLQPEDFGSYYCQHHYGTPPYTFG
GGTKLEIK,
and
anti B7-H3 NE97 hybridoma VH sequence comprises or
consists of
(SEQ ID NO: 7)
EVQLVESGGDLVKPGGSLKLSCAASGFTFSSYGMSWVRQTPDKRLEWV
ATINSGGSYIYYPDSVKGRFTISRDNAENTLYLQMSSLKSEDTAMYYCAR
HEGLPLDYWGQGTTLTVSS.

3) Anti-B7-H3 chimeric antigen receptor according to anyone of claims 1-2, wherein the linker which links anti B7-H3 NE97 hybridoma VL sequence and anti B7-H3 NE97 hybridoma VH sequence is a short flexible linker glycines-rich with a length from 7 to 14 amino acids, such as from 7 to 12, from 7 to 10 or 8 amino acids, such as for example (G4S)2 linker GGGGSGGGG (SEQ ID NO:8), G4SG2 linker GGGGSGG (SEQ ID NO:9) or G3SG4 linker GGGSGGGG (SEQ ID NO:10) SG4SG3 linker SGGGGSGGG (SEQ ID NO:54), (SG4)2 S linker SGGGGSGGGGS (SEQ ID NO:55), (SG4)2 SG linker SGGGGSGGGGSG (SEQ ID NO:56), (SG4)2 SG3 linker SGGGGSGGGGSGGG linker (SEQ ID NO:57), (SG4)2 SGGGGSGGGG (SEQ ID NO:58), (SG4)2 SG2 SGGGGSGGGGSGG (SEQ ID NO:59), preferably, G3SG4 linker.

4) Anti-B7-H3 chimeric antigen receptor according to anyone of claims 1-3, wherein said hinge comprises or consists of one or more of the following hinges:
```
hinge Spacer-CD8α
(SEQ ID NO: 11)
PAPRPPTPAPTIASQPLSLRPEACRPAAGGAVHTRGLDFA;
CD8stalk:
  (SEQ ID NO: 12)
TTTPAPRPPTPAPTIASQPLSLRPEACRPAAGGAVHTRGLDFACD;
Hinge CD28
  (SEQ ID NO: 13)
EVMYPPPYLDNEKSNGTIIHVKGKHLCPSPLFPGPSKP;
hinge CH2-CH3
  (SEQ ID NO: 14)
ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK;
or
hinge CH3:
  (SEQ ID NO: 15)
ESKYGPPCPSCPGQPREPQVYTLPPSQEEMTKNQVSLTCLVKGFYPSDIA
VEWESNGQPENNYKTTPPVLDSDGSFFLYSRLTVDKSRWQEGNVFSCSVM
HEALHNHYTQKSLSLSLGK,
preferably hinge Spacer-CD8α
(SEQ ID NO: 11)
PAPRPPTPAPTIASQPLSLRPEACRPAAGGAVHTRGLDFA.
```

5) Anti-B7-H3 chimeric antigen receptor according to anyone of claims 1-4, wherein said hinge is linked, at the N terminus, to a trackable marker, said trackable marker being linked, optionally by a second linker, to the anti B7-H3 single chain antibody domain.

6) Anti-B7-H3 chimeric antigen receptor according to claim 5, wherein
the trackable marker is chosen from the group consisting of:
```
ΔCD34:
  (SEQ ID NO: 16)
ELPTQGTFSNVSTNVS;
ΔCD19:
(SEQ ID. NO: 17)
PEEPLVVKVEEGDNAVLQCLKGTSDGPTQQLTWSRESPLKPFLK
LSLGLPGLGIHMRPLAIWLFIFNVSQQMGGFYLCQPGPPSEKAWQPGWTV
NVEGSGELFRWNVSDLGGLGCGLKNRSSEGPSSPSGKLMSPKLYVWAKDR
PEIWEGEPPCLPPRDSLNQSLSQDLTMAPGSTLWLSCGVPPDSVSRGPLS
WTHVHPKGPKSLLSLELKDDRPARDMWVMETGLLLPRATAQDAGKYYCHR
GNLTMSFHLEITARPVLWHWLLRTGGWK;
NGFR:
(SEQ ID NO: 18)
KEACPTGLYTHSGECCKACNLGEGVAQPCGANQTVCEPCLDSVT
FSDVVSATEPCKPCTECVGLQSMSAPCVEADDAVCRCAYGYYQDETTGRC
EACRVCEAGSGLVFSCQDKQNTVCEECPDGTYSDEANHVDPCLPCTVCED
TERQLRECTRWADAECEEIPGRWITRSTPPEGSDSTAPSTQEPEAPPEQD
LIASTVAGVVTTVMGSSQPVVTRGTTDN;
preferably
ΔCD34:
  (SEQ ID NO: 16)
ELPTQGTFSNVSTNVS.
```

7) Anti-B7-H3 chimeric antigen receptor according to anyone of claims 1-6, wherein the trans membrane domain is chosen from the group consisting of CD8aTM: CDIYIWAPLAGTCGVLLLSLVIT (SEQ ID NO:19); CD28TM: FWVLVVVGGVLACYSLLVTVAFIIFWV (SEQ ID NO:20); preferably CD8aTM: CDIYIWAPLAGTCGVLLLSLVIT (SEQ ID NO:19).

8) Anti-B7-H3 chimeric antigen receptor according to anyone of claims 1-7, wherein the co-stimulatory signaling domain is chosen from the group consisting of
```
CD28 cytoplasmic sequence:
(SEQ ID NO: 21)
RSKRSRLLHSDYMNMTPRRPGPTRKHYQPYAPPRDFAAYRS,
CD137 (4-1BB) sequence:
  (SEQ ID NO: 22)
KRGRKKLLYIFKQPFMRPVQTTQEEDGCSCRFPEEEEGGCEL,
OX40 sequence:
  (SEQ ID NO: 23)
RDQRLPPDAHKPPGGGSFRTPIQEEQADAHSTLAKI,
a sequence obtained by linking
CD28 cytoplasmic sequence:
(SEQ ID NO: 21)
RSKRSRLLHSDYMNMTPRRPGPTRKHYQPYAPPRDFAAYRS
to
CD137 (4-1BB) sequence:
(SEQ ID NO: 22)
KRGRKKLLYIFKQPFMRPVQTTQEEDGCSCRFPEEEEGGCEL,
or
a sequence obtained by linking
CD28 cytoplasmic sequence:
(SEQ ID NO: 21)
RSKRSRLLHSDYMNMTPRRPGPTRKHYQPYAPPRDFAAYRS,
to
OX40 sequence:
  (SEQ ID NO: 23)
RDQRLPPDAHKPPGGGSFRTPIQEEQADAHSTLAKI.
9) Anti-B7-H3 chimeric antigen receptor
according to anyone of claims 1-8, wherein
CD3-Zeta chain is
  (SEQ ID NO: 24)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR*.
```

10) Anti-B7-H3 chimeric antigen receptor according to anyone of claims 1-9, further comprising cytoplasmic moiety of CD8cyt, CD8a cytoplasmic (CD8a cyto):
LYCNHRN (SEQ ID NO:25) between the trans membrane domain and the co-stimulatory signaling domain.

11) Anti-B7-H3 chimeric antigen receptor according to anyone of claims 1-10, wherein the signal peptide comprises or consists of MEFGLSWLFLVAILKGVQC (SEQ ID NO:26).

12) Anti-B7-H3 chimeric antigen receptor according to anyone of claims 1-11, wherein said anti-B7-H3 chimeric antigen receptor comprises or consists of the following sequence:
(SEQ ID NO: 27)
MEFGLSWLFLVAILKGVQCSRDIQMTQSPASLSASVGETVTITCRASEII
YSYLAWYQQKQGKSPQLLVYNAKTLVEGVPSRFSGSGSGTQFSLKINSLQ
PEDFGSYYCQHHYGTPPYTFGGGTKLEIKGGGSGGGGEVOLVESGGDLVK
PGGSLKLSCAASGFTFSSYGMSWVRQTPDKRLEWVATINSGGSYIYYPDS
VKGRFTISRDNAENTLYLQMSSLKSEDTAMYYCARHEGLPLDYWGQGTTL
TVSSACELPTQGTFSNVSTNVSPAPRPPTPAPTIASQPLSLRPEACRPAA
GGAVHTRGLDFACDIYIWAPLAGTCGVLLLSLVITLYCNHRNEFRSKRSR
LLHSDYMNMTPRRPGPTRKHYQPYAPPRDFAAYRSKRGRKKLLYIFKQPF
MRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR*
Or
(SEQ ID NO: 28)
MEFGLSWLFLVAILKGVQCSRDIQMTQSPASLSASVGETVTITCRASEII
YSYLAWYQQKQGKSPQLLVYNAKTLVEGVPSRFSGSGSGTQFSLKINSLQ
PEDFGSYYCQHHYGTPPYTFGGGTKLEIKGGGSGGGGEVOLVESGGDLVK
PGGSLKLSCAASGFTFSSYGMSWVRQTPDKRLEWVATINSGGSYIYYPDS
VKGRFTISRDNAENTLYLQMSSLKSEDTAMYYCARHEGLPLDYWGQGTTL
TVSSACELPTQGTFSNVSTNVSPAPRPPTPAPTIASQPLSLRPEACRPAA
GGAVHTRGLDFACDIYIWAPLAGTCGVLLLSLVITLYCNHRNEFRSKRSR
LLHSDYMNMTPRRPGPTRKHYQPYAPPRDFAAYRSRDQRLPPDAHKPPGG
GSFRTPIQEEQADAHSTLAKIRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR*.

13) Nucleotide sequence comprising or consisting of a nucleotide sequence which encodes an anti-B7-H3 chimeric antigen receptor according to anyone of claims 1-12.

14) Nucleotide sequence according to claim 13, wherein
anti B7-H3 NE97 hybridoma VL sequence is encoded
by the nucleotide sequence
(SEQ ID NO: 29)
GACATCCAGATGACTCAGTCTCCAGCCTCCCTATCTGCATCTGTGGGAGA
AACTGTCACCATCACATGTCGAGCAAGTGAGATTATTTACAGTTATTTAG
CATGGTATCAGCAGAAACAGGGAAAATCTCCTCAGCTCCTGGTCTATAAT
GCAAAAACCTTAGTAGAAGGTGTGCCATCAAGGTTCAGTGGCAGTGGATC
AGGCACACAGTTTTCTCTGAAGATCAACAGCCTGCAGCCTGAAGATTTTG
GGAGTTATTACTGTCAACATCATTATGGTACTCCTCCATACACGTTCGGA
GGGGGGACCAAGCTGGAAATAAAA,
and
anti B7-H3 NE97 hybridoma VH sequence is encoded
by the nucleotide sequence
(SEQ ID NO: 30)
GAGGTGCAGCTGGTGGAGTCTGGGGGAGACTTAGTGAAGCCTGGAGGGTC
CCTGAAACTCTCCTGTGCAGCCTCTGGATTCACTTTCAGTAGCTATGGCA
TGTCTTGGGTTCGCCAGACTCCAGACAAGAGGCTGGAGTGGGTCGCAACC
ATTAATAGTGGTGGTAGTTACATCTACTATCCAGACAGTGTGAAGGGGCG
ATTCACCATCTCCAGAGACAATGCCGAGAACACCCTGTACCTGCAAATGA
GCAGTCTGAAGTCTGAAGACACAGCCATGTATTACTGTGCAAGACATGAA
GGGTTACCCCTTGACTACTGGGGCCAAGGCACCACTCTCACAGTCTCCTC
A

15) Nucleotide sequence according to any one of claims 13-14, wherein the nucleotide sequence encoding anti-B7-H3 chimeric antigen receptor is:
(SEQ ID NO: 36)
ATGGAGTTTGGGCTCTCCTGGCTCTTCCTGGTCGCGATTCTGAAGGGGGTCC
AGTGTTCACGAGACATCCAGATGACTCAGTCTCCAGCCTCCCTATCTGCATCT
GTGGGAGAAACTGTCACCATCACATGTCGAGCAAGTGAGATTATTTACAGTTA
TTTAGCATGGTATCAGCAGAAACAGGGAAAATCTCCTCAGCTCCTGGTCTATA
ATGCAAAAACCTTAGTAGAAGGTGTGCCATCAAGGTTCAGTGGCAGTGGATC
AGGCACACAGTTTTCTCTGAAGATCAACAGCCTGCAGCCTGAAGATTTTGGG
AGTTATTACTGTCAACATCATTATGGTACTCCTCCATACACGTTCGGAGGGGG
GACCAAGCTGGAAATAAAAGGCGGAGGTTCAGGCGGAGGAGGGGAGGTGC
AGCTGGTGGAGTCTGGGGGAGACTTAGTGAAGCCTGGAGGGTCCCTGAAAC
TCTCCTGTGCAGCCTCTGGATTCACTTTCAGTAGCTATGGCATGTCTTGGGTT
CGCCAGACTCCAGACAAGAGGCTGGAGTGGGTCGCAACCATTAATAGTGGT
GGTAGTTACATCTACTATCCAGACAGTGTGAAGGGGCGATTCACCATCTCCA
GAGACAATGCCGAGAACACCCTGTACCTGCAAATGAGCAGTCTGAAGTCTGA
AGACACAGCCATGTATTACTGTGCAAGACATGAAGGGTTACCCCTTGACTACT
GGGGCCAAGGCACCACTCTCACAGTCTCCTCAGCATGCGAACTTCCTACTCA
GGGGACTTTCTCAAACGTTAGCACAAACGTAAGTCCCGCCCCAAGACCCCCC
ACACCTGCGCCGACCATTGCTTCTCAACCCCTGAGTTTGAGACCCGAGGCCT
GCCGGCCAGCTGCCGGCGGGGCCGTGCATACAAGAGGACTCGATTTCGCTT
GCGACATCTACATCTGGGCTCCCCTCGCTGGCACCTGTGGGGTGCTGCTGC
TGTCACTCGTGATCACCCTTTATTGCAACCATCGAAACGAATTCAGAAGTAAA
CGGTCAAGGCTTCTGCACAGCGATTATATGAATATGACACCAAGAAGACCTG
GTCCAACCCGGAAACACTATCAGCCCTACGCGCCCCCTAGAGACTTCGCAGC
ATACCGCTCTAAGAGAGGGAGAAAAAAATTGCTCTATATTTTTAAACAACCATT
TATGAGGCCCGTACAGACAACTCAGGAAGAGGATGGCTGTAGTTGCCGCTTC
CCAGAGGAGGAGGAAGGAGGCTGCGAGTTGAGAGTTAAATTCAGTAGAAGT
GCGGATGCGCCTGCTTACCAGCAGGGCCAGAACCAACTGTACAATGAACTGA
ATCTCGGGCGCCGAGAAGAGTATGACGTCCTCGATAAGCGGAGGGGTAGGG
ATCCTGAAATGGGTGGGAAGCCAAGAAGAAAAAACCCCCAGGAAGGACTGTA
TAACGAACTTCAGAAGGACAAGATGGCAGAGGCCTACTCTGAGATTGGCATG
AAAGGCGAACGACGGCGCGGTAAAGGTCATGACGGGCTGTACCAGGGCCTG
TCCACAGCGACGAAGGACACTTACGACGCCCTGCACATGCAGGCACTCCCC
CCCAGGTGA
or
(SEQ ID NO: 37)
ATGGAGTTTGGGCTCTCCTGGCTCTTCCTGGTCGCGATTCTGAAGGGGGTCC
AGTGTTCACGAGACATCCAGATGACTCAGTCTCCAGCCTCCCTATCTGCATCT
GTGGGAGAAACTGTCACCATCACATGTCGAGCAAGTGAGATTATTTACAGTTA
TTTAGCATGGTATCAGCAGAAACAGGGAAAATCTCCTCAGCTCCTGGTCTATA
ATGCAAAAACCTTAGTAGAAGGTGTGCCATCAAGGTTCAGTGGCAGTGGATC
AGGCACACAGTTTTCTCTGAAGATCAACAGCCTGCAGCCTGAAGATTTTGGG
AGTTATTACTGTCAACATCATTATGGTACTCCTCCATACACGTTOGGAGGGGG
GACCAAGCTGGAAATAAAAGGCGGAGGTTCAGGCGGAGGAGGGGAGGTGC
AGCTGGTGGAGTCTGGGGGAGACTTAGTGAAGCCTGGAGGGTCCCTGAAAC
TCTCCTGTGCAGCCTCTGGATTCACTTTCAGTAGCTATGGCATGTCTTGGGTT
CGCCAGACTCCAGACAAGAGGCTGGAGTGGGTCGCAACCATTAATAGTGGT
GGTAGTTACATCTACTATCCAGACAGTGTGAAGGGGCGATTCACCATCTCCA
GAGACAATGCCGAGAACACCCTGTACCTGCAAATGAGCAGTCTGAAGTCTGA
AGACACAGCCATGTATTACTGTGCAAGACATGAAGGGTTACCCCTTGACTACT
GGGGCCAAGGCACCACTCTCACAGTCTCCTCAGCATGCGAACTTCCTACTCA
GGGGACTTTCTCAAACGTTAGCACAAACGTAAGTCCCGCCCCAAGACCCCCC
ACACCTGCGCCGACCATTGCTTCTCAACCCCTGAGTTTGAGACCCGAGGCCT
GCCGGCCAGCTGCCGGCGGGGCCGTGCATACAAGAGGACTCGATTTCGCTT
GCGACATCTACATCTGGGCTCCCCTCGCTGGCACCTGTGGGGTGCTGCTGC
TGTCACTCGTGATCACCCTTTATTGCAACCATCGAAACGAATTCAGAAGTAAA
CGGTCAAGGCTTCTGCACAGCGATTATATGAATATGACACCAAGAAGACCTG
GTCCAACCCGGAAACACTATCAGCCCTACGCGCCCCCTAGAGACTTCGCAGC
ATACCGCTCTCGCGATCAAAGACTCCCGCCCGATGCCCACAAACCCCCTGGC
GGGGGCAGCTTTAGGACACCCATTCAAGAAGAGCAGGCAGACGCCCACAGC
ACCTTGGCCAAAATTAGAGTTAAATTCAGTAGAAGTGCGGATGCGCCTGCTTA
CCAGCAGGGCCAGAACCAACTGTACAATGAACTGAATCTCGGGCGCCGAGA
AGAGTATGACGTCCTCGATAAGCGGAGGGGTAGGGATCCTGAAATGGGTGG
GAAGCCAAGAAGAAAAAACCCCCAGGAAGGACTGTATAACGAACTTCAGAAG
GACAAGATGGCAGAGGCCTACTCTGAGATTGGCATGAAAGGCGAACGACGG
CGCGGTAAAGGTCATGACGGGCTGTACCAGGGCCTGTCCACAGCGACGAAG
GACACTTACGACGCCCTGCACATGCAGGCACTCCCCCCCAGGTGA.

16) Nucleotide sequence according to any one of claims 13-15, said nucleotide sequence further comprising a nucleotide sequence encoding a suicide gene inducible amino acid sequence linked to the nucleotide sequence encoding said chimeric antigen receptor by a nucleotide sequence encoding a 2A self-cleaving peptide.

17) Nucleotide sequence according to claim 16, wherein the suicide gene inducible amino acid sequence is a chimeric Caspase-9 polypeptide or comprises a herpes simplex virus thymidine kinase.

18) Nucleotide sequence according to anyone of claims 16-17, which is
(SEQ ID NO: 38)
ATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTTTAT
GCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGT
GCCACTGGGCACCCAGGCATCATCCCACCACATGCCACTCTCGTCTTCGATG
TGGAGCTTCTAAAACTGGAATCTGGCGGTGGATCCGGAGTCGACGGATTTGG
TGATGTCGGTGCTCTTGAGAGTTTGAGGGGAAATGCAGATTTGGCTTACATC
CTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGA
AGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGG
ACCACGGTGCTCTGGACTGCTGCGTGGTGGTCATTCTCTCTCACGGCTGTCA
GGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGC
CTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTG
GCAGTAACCCCGAGCCAGATGCCACCCCGTTCCAGGAAGGTTTGAGGACCT
TCGACCAGCTGGACGCCATATCTAGTTTGCCCACACCCAGTGACATCTTTGT
GTCCTACTCTACTTTCCCAGGTTTTGTTTCCTGGAGGGACCCCAAGAGTGGC
TCCTGGTACGTTGAGACCCTGGACGACATCTTTGAGCAGTGGGCTCACTCTG
AAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTT
TAAAACATCAGCTAGCAGAGCCGAGGGCAGGGGAAGTCTTCTAACATGCGG
GGACGTGGAGGAAAATCCCGGGCCCATGGAGTTTGGGCTCTCCTGGCTCTT
CCTGGTCGCGATTCTGAAGGGGGTCCAGTGTTCACGAGACATCCAGATGACT
CAGTCTCCAGCCTCCCTATCTGCATCTGTGGGAGAAACTGTCACCATCACAT
GTCGAGCAAGTGAGATTATTTACAGTTATTTAGCATGGTATCAGCAGAAACAG
GGAAAATCTCCTCAGCTCCTGGTCTATAATGCAAAAACCTTAGTAGAAGGTGT
GCCATCAAGGTTCAGTGGCAGTGGATCAGGCACACAGTTTTCTCTGAAGATC
AACAGCCTGCAGCCTGAAGATTTTGGGAGTTATTACTGTCAACATCATTATGG
TACTCCTCCATACACGTTCGGAGGGGGGACCAAGCTGGAAATAAAAGGCGG
AGGTTCAGGCGGAGGAGGGGAGGTGCAGCTGGTGGAGTCTGGGGGAGACT
TAGTGAAGCCTGGAGGGTCCCTGAAACTCTCCTGTGCAGCCTCTGGATTCAC
TTTCAGTAGCTATGGCATGTCTTGGGTTCGCCAGACTCCAGACAAGAGGCTG
GAGTGGGTCGCAACCATTAATAGTGGTGGTAGTTACATCTACTATCCAGACA
GTGTGAAGGGGCGATTCACCATCTCCAGAGACAATGCCGAGAACACCCTGTA
CCTGCAAATGAGCAGTCTGAAGTCTGAAGACACAGCCATGTATTACTGTGCA
AGACATGAAGGGTTACCCCTTGACTACTGGGGCCAAGGCACCACTCTCACAG
TCTCCTCAGCATGCGAACTTCCTACTCAGGGGACTTTCTCAAACGTTAGCACA
AACGTAAGTCCCGCCCCAAGACCCCCCACACCTGCGCCGACCATTGCTTCTC
AACCCCTGAGTTTGAGACCCGAGGCCTGCCGGCCAGCTGCCGGGGGGCC
GTGCATACAAGAGGACTCGATTTCGCTTGCGACATCTACATCTGGGCTCCCC
TCGCTGGCACCTGTGGGGTGCTGCTGCTGTCACTCGTGATCACCCTTTATTG
CAACCATCGAAACGAATTCAGAAGTAAACGGTCAAGGCTTCTGCACAGCGAT
TATATGAATATGACACCAAGAAGACCTGGTCCAACCCGGAAACACTATCAGC
CCTACGCGCCCCCTAGAGACTTCGCAGCATACCGCTCTAAGAGAGGGAGAA
AAAAATTGCTCTATATTTTTAAACAACCATTTATGAGGCCCGTACAGACAACTC
AGGAAGAGGATGGCTGTAGTTGCCGCTTCCCAGAGGAGGAGGAAGGAGGCT
GCGAGTTGAGAGTTAAATTCAGTAGAAGTGCGGATGCGCCTGCTTACCAGCA
GGGCCAGAACCAACTGTACAATGAACTGAATCTCGGGCGCCGAGAAGAGTAT
GACGTCCTCGATAAGCGGAGGGGTAGGGATCCTGAAATGGGTGGGAAGCCA
AGAAGAAAAAACCCCCAGGAAGGACTGTATAACGAACTTCAGAAGGACAAGA
TGGCAGAGGCCTACTCTGAGATTGGCATGAAAGGCGAACGACGGCGCGGTA
AAGGTCATGACGGGCTGTACCAGGGCCTGTCCACAGCGACGAAGGACACTT
ACGACGCCCTGCACATGCAGGCACTCCCCCCCAGGTGA
or
(SEQ ID NO: 39)
ATGCTCGAGGGAGTGCAGGTGGAAACCATCTCCCCAGGAGACGGGCGCACC
TTCCCCAAGCGCGGCCAGACCTGCGTGGTGCACTACACCGGGATGCTTGAA
GATGGAAAGAAAGTTGATTCCTCCCGGGACAGAAACAAGCCCTTTAAGTTTAT
GCTAGGCAAGCAGGAGGTGATCCGAGGCTGGGAAGAAGGGGTTGCCCAGAT
GAGTGTGGGTCAGAGAGCCAAACTGACTATATCTCCAGATTATGCCTATGGT
GCCACTGGGCACCCAGGCATCATCCCACCACATGCCACTCTCGTCTTCGATG
TGGAGCTTCTAAAACTGGAATCTGGCGGTGGATCCGGAGTCGACGGATTTGG
TGATGTCGGTGCTCTTGAGAGTTTGAGGGGAAATGCAGATTTGGCTTACATC
CTGAGCATGGAGCCCTGTGGCCACTGCCTCATTATCAACAATGTGAACTTCT
GCCGTGAGTCCGGGCTCCGCACCCGCACTGGCTCCAACATCGACTGTGAGA
AGTTGCGGCGTCGCTTCTCCTCGCTGCATTTCATGGTGGAGGTGAAGGGCG
ACCTGACTGCCAAGAAAATGGTGCTGGCTTTGCTGGAGCTGGCGCAGCAGG
ACCACGGTGCTCTGGACTGCTGCGTGGTGGTCATTCTCTCTCACGGCTGTCA
GGCCAGCCACCTGCAGTTCCCAGGGGCTGTCTACGGCACAGATGGATGCCC
TGTGTCGGTCGAGAAGATTGTGAACATCTTCAATGGGACCAGCTGCCCCAGC
CTGGGAGGGAAGCCCAAGCTCTTTTTCATCCAGGCCTGTGGTGGGGAGCAG
AAAGACCATGGGTTTGAGGTGGCCTCCACTTCCCCTGAAGACGAGTCCCCTG
GCAGTAACCCCGAGCCAGATGCCACCCCGTTCCAGGAAGGTTTGAGGACCT
TCGACCAGCTGGACGCCATATCTAGTTTGCCCACACCCAGTGACATCTTTGT
GTCCTACTCTACTTTCCCAGGTTTTGTTTCCTGGAGGGACCCCAAGAGTGGC
TCCTGGTACGTTGAGACCCTGGACGACATCTTTGAGCAGTGGGCTCACTCTG
AAGACCTGCAGTCCCTCCTGCTTAGGGTCGCTAATGCTGTTTCGGTGAAAGG
GATTTATAAACAGATGCCTGGTTGCTTTAATTTCCTCCGGAAAAAACTTTTCTT
TAAAACATCAGCTAGCAGAGCCGAGGGCAGGGGAAGTCTTCTAACATGCGG
GGACGTGGAGGAAAATCCCGGGCCCATGGAGTTTGGGCTCTCCTGGCTCTT
CCTGGTCGCGATTCTGAAGGGGGTCCAGTGTTCACGAGACATCCAGATGACT
CAGTCTCCAGCCTCCCTATCTGCATCTGTGGGAGAAACTGTCACCATCACAT
GTCGAGCAAGTGAGATTATTTACAGTTATTTAGCATGGTATCAGCAGAAACAG
GGAAAATCTCCTCAGCTCCTGGTCTATAATGCAAAAACCTTAGTAGAAGGTGT
GCCATCAAGGTTCAGTGGCAGTGGATCAGGCACACAGTTTTCTCTGAAGATC
AACAGCCTGCAGCCTGAAGATTTTGGGAGTTATTACTGTCAACATCATTATGG
TACTCCTCCATACACGTTCGGAGGGGGGACCAAGCTGGAAATAAAAGGCGG
AGGTTCAGGCGGAGGAGGGGAGGTGCAGCTGGTGGAGTCTGGGGGAGACT
TAGTGAAGCCTGGAGGGTCCCTGAAACTCTCCTGTGCAGCCTCTGGATTCAC
TTTCAGTAGCTATGGCATGTCTTGGGTTCGCCAGACTCCAGACAAGAGGCTG
GAGTGGGTCGCAACCATTAATAGTGGTGGTAGTTACATCTACTATCCAGACA
GTGTGAAGGGGCGATTCACCATCTCCAGAGACAATGCCGAGAACACCCTGTA
CCTGCAAATGAGCAGTCTGAAGTCTGAAGACACAGCCATGTATTACTGTGCA
AGACATGAAGGGTTACCCCTTGACTACTGGGGCCAAGGCACCACTCTCACAG
TCTCCTCAGCATGCGAACTTCCTACTCAGGGGACTTTCTCAAACGTTAGCACA
AACGTAAGTCCCGCCCCAAGACCCCCCACACCTGCGCCGACCATTGCTTCTC
AACCCCTGAGTTTGAGACCCGAGGCCTGCCGGCCAGCTGCCGGGGGGCC
GTGCATACAAGAGGACTCGATTTCGCTTGCGACATCTACATCTGGGCTCCCC
TCGCTGGCACCTGTGGGGTGCTGCTGCTGTCACTCGTGATCACCCTTTATTG
CAACCATCGAAACGAATTCAGAAGTAAACGGTCAAGGCTTCTGCACAGCGAT
TATATGAATATGACACCAAGAAGACCTGGTCCAACCCGGAAACACTATCAGC
CCTACGCGCCCCCTAGAGACTTCGCAGCATACCGCTCTCGCGATCAAAGACT
CCCGCCCGATGCCCACAAACCCCCTGGGGGGGGCAGCTTTAGGACACCCAT
TCAAGAAGAGCAGGCAGACGCCCACAGCACCTTGGCCAAAATTAGAGTTAAA
TTCAGTAGAAGTGCGGATGCGCCTGCTTACCAGCAGGGCCAGAACCAACTGT
ACAATGAACTGAATCTCGGGCGCCGAGAAGAGTATGACGTCCTCGATAAGCG
GAGGGGTAGGGATCCTGAAATGGGTGGGAAGCCAAGAAGAAAAAACCCCCA
GGAAGGACTGTATAACGAACTTCAGAAGGACAAGATGGCAGAGGCCTACTCT
GAGATTGGCATGAAAGGCGAACGACGGCGCGGTAAAGGTCATGACGGGCTG
TACCAGGGCCTGTCCACAGCGACGAAGGACACTTACGACGCCCTGCACATG
CAGGCACTCCCCCCCAGGTGA

19) Vector comprising the nucleotide sequence according to anyone of claims 13-18, wherein said vector is a DNA vector, a RNA vector, a plasmid, a lentivirus vector, adenoviral vector, retrovirus vector, such as γ-retroviral vector, or non-viral vector.

20) Cell, such as T cell, such as alfa/beta and gamma/delta T cell, NK cells, NK-T cells as well as macrophages or monocyte cells, comprising the anti-B7-H3 chimeric antigen receptor according to anyone of claims 1-12 and/or the vector or plasmid according to claim 19.

21) Cell according to claim 20, further comprising a suicide gene inducible amino acid sequence such as a chimeric Caspase-9 polypeptide or a herpes simplex virus thymidine kinase (HSV-TK) as a safety switch.

22) Cell according to claim 21, wherein the chimeric Caspase-9 polypeptide comprises or consists of:
an inducible Caspase 9 (iC9):
MLEGVQVETISPGDGRTFPKRGQTCVVHYTGMLEDGKKVDSSRDRNKPFKFML GKQEVIRGWEEGVAQMSVGQRAKLTISPDYAYGATGHPGIIPPHATLVFDVELLK LESGGGSGVDGFGDVGALESLRGNADLAYILSMEPCGHCLIINNVNFCRESGLR TRTGSNIDCEKLRRRFSSLHFMVEVKGDLTAKKMVLALLELAQQDHGALDCCVV VILSHGCQASHLQFPGAVYGTDGCPVSVEKIVNIFNGTSCPSLGGKPKLFFIQAC GGEQKDHGFEVASTSPEDESPGSNPEPDATPFQEGLRTFDQLDAISSLPTPSDIF VSYSTFPGFVSWRDPKSGSWYVETLDDIFEQWAHSEDLQSLLLRVANAVSVKGI YKQMPGCFNFLRKKLFFKTSAS (SEQ ID NO:52), which is linked by a linker, such as RA, to:
2A self-cleaving peptides T2A
(SEQ ID NO: 53)
EGRGSLLTCGDVEENPGP.

23) Cell according to any one of claims 20-22, which is obtained in culture conditions wherein both or each of IL-7 and/or IL-15 are present, for example in the culture conditions of the activation step, transduction step and/or expansion step of the process for the preparation of said cell.

24) Pharmaceutical composition comprising the nucleotide sequence according to claims 13-18, or the vector according to claim 19, or the cell according to claims 20-23 together with one or more excipients and/or adjuvants.

25) Anti B7-H3 chimeric antigen receptor according to anyone of claims 1-12, nucleotide sequence according to anyone of claims 13-18, vector according to claim 19, cell according to claims 20-23, pharmaceutical composition according to claim 24, for medical use.

26) Anti B7-H3 chimeric antigen receptor according to anyone of claims 1-12, nucleotide sequence according to anyone of claims 13-18, vector according to claim 19, cell according to claims 20-23, pharmaceutical composition according to claim 24, for use in the treatment of hematologic malignancies, such as for example Chronic Myeloid Leukemia (CML), Myelodysplastic syndromes (MDS), Acute Myeloid Leukemia (AML), Chronic lymphocytic leukemia (CLL), B cell Acute lymphoblastic leukemia (B-ALL), T cell Acute lymphoblastic leukemia (T-ALL), lymphomas (Non-Hodgkin's Lymphoma or Hodgkin's Lymphoma), Multiple Myeloma, and solid B7H3+ tumour, such as for example Neuroblastoma, retinoblastoma, sarcoma, Ewing's sarcoma, rhabdomyosarcoma, Osteosarcoma, Desmoplastic Small Round Cell Tumors (DSRCT), pancreatic cancer, Colon cancer, breast cancers, germinal carcinoma, lung carcinoma, liver carcinoma, kidney carcinoma, melanoma and brain tumors such as medulloblastoma and glioblastoma, and autoimmune diseases.


SEQUENCE LISTING (59 sequences)

SEQ ID NO: 1 - protein, 6 aa, Homo sapiens
EIIYSY

SEQ ID NO: 2 - protein, 10 aa, Homo sapiens
QHHYGTPPYT

SEQ ID NO: 3 - protein, 8 aa, Homo sapiens
GFTFSSYG

SEQ ID NO: 4 - protein, 8 aa, Homo sapiens
INSGGSYI

SEQ ID NO: 5 - protein, 10 aa, Homo sapiens
ARHEGLPLDY

SEQ ID NO: 6 - protein, 108 aa, Homo sapiens
DIQMTQSPASLSASVGETVTITCRASEIIYSYLAWYQQKQGKSPQLLVYNAKTLVEGVPSRFSGSGSGTQFSLKINSLQPEDFGSYYCQHHYGTPPYTFGGGTKLEIK

SEQ ID NO: 7 - protein, 117 aa, Homo sapiens
EVQLVESGGDLVKPGGSLKLSCAASGFTFSSYGMSWVRQTPDKRLEWVATINSGGSYIYYPDSVKGRFTISRDNAENTLYLQMSSLKSEDTAMYYCARHEGLPLDYWGQGTTLTVSS

SEQ ID NO: 8 - protein, 9 aa, synthetic construct
  REGION 1..9: note = linker
GGGGSGGGG

SEQ ID NO: 9 - protein, 7 aa, synthetic construct
  REGION 1..7: note = linker
GGGGSGG

SEQ ID NO: 10 - protein, 8 aa, synthetic construct
  REGION 1..8: note = linker
GGGSGGGG

SEQ ID NO: 11 - protein, 40 aa, synthetic construct
  REGION 1..40: note = hinge Spacer-CD8 alpha
PAPRPPTPAPTIASQPLSLRPEACRPAAGGAVHTRGLDFA

SEQ ID NO: 12 - protein, 45 aa, synthetic construct
  REGION 1..45: note = CD8stalk
TTTPAPRPPTPAPTIASQPLSLRPEACRPAAGGAVHTRGLDFACD

SEQ ID NO: 13 - protein, 38 aa, synthetic construct
  REGION 1..38: note = Hinge CD28
EVMYPPPYLDNEKSNGTIIHVKGKHLCPSPLFPGPSKP

SEQ ID NO: 14 - protein, 229 aa, synthetic construct
  REGION 1..229: note = hinge CH2-CH3
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 119 aa, synthetic construct
  REGION 1..119: note = hinge CH3
ESKYGPPCPSCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 16 aa, synthetic construct
  REGION 1..16: note = DELTA CD34 trackable marker
ELPTQGTFSNVSTNVS

SEQ ID NO: 17 - protein, 272 aa, synthetic construct
  REGION 1..272: note = DELTA CD19 trackable marker
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 18 - protein, 222 aa, synthetic construct
  REGION 1..222: note = NGFR trackable marker
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDN

SEQ ID NO: 19 - protein, 23 aa, synthetic construct
  REGION 1..23: note = CD8aTM trans membrane domain
CDIYIWAPLAGTCGVLLLSLVIT

SEQ ID NO: 20 - protein, 27 aa, synthetic construct
  REGION 1..27: note = CD28TM trans membrane domain
FWVLVVVGGVLACYSLLVTVAFIIFWV

SEQ ID NO: 21 - protein, 41 aa, synthetic construct
  REGION 1..41: note = CD28 cytoplasmic sequence
RSKRSRLLHSDYMNMTPRRPGPTRKHYQPYAPPRDFAAYRS

SEQ ID NO: 22 - protein, 42 aa, synthetic construct
  REGION 1..42: note = CD137 (4-1BB) sequence
KRGRKKLLYIFKQPFMRPVQTTQEEDGCSCRFPEEEEGGCEL

SEQ ID NO: 23 - protein, 36 aa, synthetic construct
  REGION 1..36: note = OX40 sequence
RDQRLPPDAHKPPGGGSFRTPIQEEQADAHSTLAKI

SEQ ID NO: 24 - protein, 112 aa, synthetic construct
  REGION 1..112: note = CD3-Zeta chain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 7 aa, synthetic construct
  REGION 1..7: note = CD8a cytoplasmic
LYCNHRN

SEQ ID NO: 26 - protein, 19 aa, synthetic construct
  REGION 1..19: note = signal peptide
MEFGLSWLFLVAILKGVQC

SEQ ID NO: 27 - protein, 539 aa, synthetic construct
  REGION 1..539: note = anti-B7-H3 chimeric antigen receptor
MEFGLSWLFLVAILKGVQCSRDIQMTQSPASLSASVGETVTITCRASEIIYSYLAWYQQKQGKSPQLLVYNAKTLVEGVPSRFSGSGSGTQFSLKINSLQPEDFGSYYCQHHYGTPPYTFGGGTKLEIKGGGSGGGGEVQLVESGGDLVKPGGSLKLSCAASGFTFSSYGMSWVRQTPDKRLEWVATINSGGSYIYYPDSVKGRFTISRDNAENTLYLQMSSLKSEDTAMYYCARHEGLPLDYWGQGTTLTVSSACELPTQGTFSNVSTNVSPAPRPPTPAPTIASQPLSLRPEACRPAAGGAVHTRGLDFACDIYIWAPLAGTCGVLLLSLVITLYCNHRNEFRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 533 aa, synthetic construct
  REGION 1..533: note = anti-B7-H3 chimeric antigen receptor
MEFGLSWLFLVAILKGVQCSRDIQMTQSPASLSASVGETVTITCRASEIIYSYLAWYQQKQGKSPQLLVYNAKTLVEGVPSRFSGSGSGTQFSLKINSLQPEDFGSYYCQHHYGTPPYTFGGGTKLEIKGGGSGGGGEVQLVESGGDLVKPGGSLKLSCAASGFTFSSYGMSWVRQTPDKRLEWVATINSGGSYIYYPDSVKGRFTISRDNAENTLYLQMSSLKSEDTAMYYCARHEGLPLDYWGQGTTLTVSSACELPTQGTFSNVSTNVSPAPRPPTPAPTIASQPLSLRPEACRPAAGGAVHTRGLDFACDIYIWAPLAGTCGVLLLSLVITLYCNHRNEFRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 324 bp, synthetic construct
  misc_feature 1..324: note = nucleotide sequence encoding anti B7-H3 NE97 hybridoma VLsequence
  source 1..324: mol_type = other DNA
gacatccagatgactcagtctccagcctccctatctgcatctgtgggagaaactgtcaccatcacatgtcgagcaagtgagattatttacagttatttagcatggtatcagcagaaacagggaaaatctcctcagctcctggtctataatgcaaaaaccttagtagaaggtgtgccatcaaggttcagtggcagtggatcaggcacacagttttctctgaagatcaacagcctgcagcctgaagattttgggagttattactgtcaacatcattatggtactcctccatacacgttcggaggggggaccaagctggaaataaaa

SEQ ID NO: 30 - DNA, 351 bp, synthetic construct
  misc_feature 1..351: note = nucleotide sequence encoding anti B7-H3 NE97 hybridoma VHsequence
  source 1..351: mol_type = other DNA
gaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattaatagtggtggtagttacatctactatccagacagtgtgaaggggcgattcaccatctccagagacaatgccgagaacaccctgtacctgcaaatgagcagtctgaagtctgaagacacagccatgtattactgtgcaagacatgaagggttaccccttgactactggggccaaggcaccactctcacagtctcctca

SEQ ID NO: 31 - DNA, 18 bp, synthetic construct
  source 1..18: mol_type = other DNA
gagattatttacagttat

SEQ ID NO: 32 - DNA, 30 bp, synthetic construct
  misc_feature 1..30: note = CDR3 of anti B7-H3 NE97 hybridoma VL sequence
  source 1..30: mol_type = other DNA
caacatcattatggtactcctccatacacg

SEQ ID NO: 33 - DNA, 24 bp, synthetic construct
  misc_feature 1..24: note = CDR1 of anti B7-H3 NE97 hybridoma VH sequence
  source 1..24: mol_type = other DNA
ggattcactttcagtagctatggc

SEQ ID NO: 34 - DNA, 24 bp, synthetic construct
  misc_feature 1..24: note = CDR2 of anti B7-H3 NE97 hybridoma VH sequence
  source 1..24: mol_type = other DNA
attaatagtggtggtagttacatc

SEQ ID NO: 35 - DNA, 30 bp, synthetic construct
  misc_feature 1..30: note = CDR3 of anti B7-H3 NE97 hybridoma VH sequence
  source 1..30: mol_type = other DNA
gcaagacatgaagggttaccccttgactac

SEQ ID NO: 36 - DNA, 1620 bp, synthetic construct
  misc_feature 1..1620: note = nucleotide sequence encoding anti-B7-H3 chimeric antigen receptor
  source 1..1620: mol_type = other DNA
atggagtttgggctctcctggctcttcctggtcgcgattctgaagggggtccagtgttcacgagacatccagatgactcagtctccagcctccctatctgcatctgtgggagaaactgtcaccatcacatgtcgagcaagtgagattatttacagttatttagcatggtatcagcagaaacagggaaaatctcctcagctcctggtctataatgcaaaaaccttagtagaaggtgtgccatcaaggttcagtggcagtggatcaggcacacagttttctctgaagatcaacagcctgcagcctgaagattttgggagttattactgtcaacatcattatggtactcctccatacacgttcggaggggggaccaagctggaaataaaaggcggaggttcaggcggaggaggggaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattaatagtggtggtagttacatctactatccagacagtgtgaaggggcgattcaccatctccagagacaatgccgagaacaccctgtacctgcaaatgagcagtctgaagtctgaagacacagccatgtattactgtgcaagacatgaagggttaccccttgactactggggccaaggcaccactctcacagtctcctcagcatgcgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctacatctgggctcccctcgctggcacctgtggggtgctgctgctgtcactcgtgatcaccctttattgcaaccatcgaaacgaattcagaagtaaacggtcaaggcttctgcacagcgattatatgaatatgacaccaagaagacctggtccaacccggaaacactatcagccctacgcgccccctagagacttcgcagcataccgctctaagagagggagaaaaaaattgctctatatttttaaacaaccatttatgaggcccgtacagacaactcaggaagaggatggctgtagttgccgcttcccagaggaggaggaaggaggctgcgagttgagagttaaattcagtagaagtgcggatgcgcctgcttaccagcagggccagaaccaactgtacaatgaactgaatctcgggcgccgagaagagtatgacgtcctcgataagcggaggggtagggatcctgaaatgggtgggaagccaagaagaaaaaacccccaggaaggactgtataacgaacttcagaaggacaagatggcagaggcctactctgagattggcatgaaaggcgaacgacggcgcggtaaaggtcatgacgggctgtaccagggcctgtccacagcgacgaaggacacttacgacgccctgcacatgcaggcactcccccccaggtga

SEQ ID NO: 37 - DNA, 1602 bp, synthetic construct
  misc_feature 1..1602: note = nucleotide sequence encoding anti-B7-H3 chimeric antigen receptor
  source 1..1602: mol_type = other DNA
atggagtttgggctctcctggctcttcctggtcgcgattctgaagggggtccagtgttcacgagacatccagatgactcagtctccagcctccctatctgcatctgtgggagaaactgtcaccatcacatgtcgagcaagtgagattatttacagttatttagcatggtatcagcagaaacagggaaaatctcctcagctcctggtctataatgcaaaaaccttagtagaaggtgtgccatcaaggttcagtggcagtggatcaggcacacagttttctctgaagatcaacagcctgcagcctgaagattttgggagttattactgtcaacatcattatggtactcctccatacacgttcggaggggggaccaagctggaaataaaaggcggaggttcaggcggaggaggggaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattaatagtggtggtagttacatctactatccagacagtgtgaaggggcgattcaccatctccagagacaatgccgagaacaccctgtacctgcaaatgagcagtctgaagtctgaagacacagccatgtattactgtgcaagacatgaagggttaccccttgactactggggccaaggcaccactctcacagtctcctcagcatgcgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctacatctgggctcccctcgctggcacctgtggggtgctgctgctgtcactcgtgatcaccctttattgcaaccatcgaaacgaattcagaagtaaacggtcaaggcttctgcacagcgattatatgaatatgacaccaagaagacctggtccaacccggaaacactatcagccctacgcgccccctagagacttcgcagcataccgctctcgcgatcaaagactcccgcccgatgcccacaaaccccctggcgggggcagctttaggacacccattcaagaagagcaggcagacgcccacagcaccttggccaaaattagagttaaattcagtagaagtgcggatgcgcctgcttaccagcagggccagaaccaactgtacaatgaactgaatctcgggcgccgagaagagtatgacgtcctcgataagcggaggggtagggatcctgaaatgggtgggaagccaagaagaaaaaacccccaggaaggactgtataacgaacttcagaaggacaagatggcagaggcctactctgagattggcatgaaaggcgaacgacggcgcggtaaaggtcatgacgggctgtaccagggcctgtccacagcgacgaaggacacttacgacgccctgcacatgcaggcactcccccccaggtga

SEQ ID NO: 38 - DNA, 2886 bp, synthetic construct
  misc_feature 1..2886: note = nucleotide sequence encoding suicide gene inducible and anti-B7-H3 chimeric antigen receptor
  source 1..2886: mol_type = other DNA
atgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagccgagggcaggggaagtcttctaacatgcggggacgtggaggaaaatcccgggcccatggagtttgggctctcctggctcttcctggtcgcgattctgaagggggtccagtgttcacgagacatccagatgactcagtctccagcctccctatctgcatctgtgggagaaactgtcaccatcacatgtcgagcaagtgagattatttacagttatttagcatggtatcagcagaaacagggaaaatctcctcagctcctggtctataatgcaaaaaccttagtagaaggtgtgccatcaaggttcagtggcagtggatcaggcacacagttttctctgaagatcaacagcctgcagcctgaagattttgggagttattactgtcaacatcattatggtactcctccatacacgttcggaggggggaccaagctggaaataaaaggcggaggttcaggcggaggaggggaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattaatagtggtggtagttacatctactatccagacagtgtgaaggggcgattcaccatctccagagacaatgccgagaacaccctgtacctgcaaatgagcagtctgaagtctgaagacacagccatgtattactgtgcaagacatgaagggttaccccttgactactggggccaaggcaccactctcacagtctcctcagcatgcgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctacatctgggctcccctcgctggcacctgtggggtgctgctgctgtcactcgtgatcaccctttattgcaaccatcgaaacgaattcagaagtaaacggtcaaggcttctgcacagcgattatatgaatatgacaccaagaagacctggtccaacccggaaacactatcagccctacgcgccccctagagacttcgcagcataccgctctaagagagggagaaaaaaattgctctatatttttaaacaaccatttatgaggcccgtacagacaactcaggaagaggatggctgtagttgccgcttcccagaggaggaggaaggaggctgcgagttgagagttaaattcagtagaagtgcggatgcgcctgcttaccagcagggccagaaccaactgtacaatgaactgaatctcgggcgccgagaagagtatgacgtcctcgataagcggaggggtagggatcctgaaatgggtgggaagccaagaagaaaaaacccccaggaaggactgtataacgaacttcagaaggacaagatggcagaggcctactctgagattggcatgaaaggcgaacgacggcgcggtaaaggtcatgacgggctgtaccagggcctgtccacagcgacgaaggacacttacgacgccctgcacatgcaggcactcccccccaggtga

SEQ ID NO: 39 - DNA, 2868 bp, synthetic construct
  misc_feature 1..2868: note = nucleotide sequence encoding suicide gene inducible andanti-B7-H3 chimeric antigen receptor
  source 1..2868: mol_type = other DNA
atgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagccgagggcaggggaagtcttctaacatgcggggacgtggaggaaaatcccgggcccatggagtttgggctctcctggctcttcctggtcgcgattctgaagggggtccagtgttcacgagacatccagatgactcagtctccagcctccctatctgcatctgtgggagaaactgtcaccatcacatgtcgagcaagtgagattatttacagttatttagcatggtatcagcagaaacagggaaaatctcctcagctcctggtctataatgcaaaaaccttagtagaaggtgtgccatcaaggttcagtggcagtggatcaggcacacagttttctctgaagatcaacagcctgcagcctgaagattttgggagttattactgtcaacatcattatggtactcctccatacacgttcggaggggggaccaagctggaaataaaaggcggaggttcaggcggaggaggggaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattaatagtggtggtagttacatctactatccagacagtgtgaaggggcgattcaccatctccagagacaatgccgagaacaccctgtacctgcaaatgagcagtctgaagtctgaagacacagccatgtattactgtgcaagacatgaagggttaccccttgactactggggccaaggcaccactctcacagtctcctcagcatgcgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctacatctgggctcccctcgctggcacctgtggggtgctgctgctgtcactcgtgatcaccctttattgcaaccatcgaaacgaattcagaagtaaacggtcaaggcttctgcacagcgattatatgaatatgacaccaagaagacctggtccaacccggaaacactatcagccctacgcgccccctagagacttcgcagcataccgctctcgcgatcaaagactcccgcccgatgcccacaaaccccctggcgggggcagctttaggacacccattcaagaagagcaggcagacgcccacagcaccttggccaaaattagagttaaattcagtagaagtgcggatgcgcctgcttaccagcagggccagaaccaactgtacaatgaactgaatctcgggcgccgagaagagtatgacgtcctcgataagcggaggggtagggatcctgaaatgggtgggaagccaagaagaaaaaacccccaggaaggactgtataacgaacttcagaaggacaagatggcagaggcctactctgagattggcatgaaaggcgaacgacggcgcggtaaaggtcatgacgggctgtaccagggcctgtccacagcgacgaaggacacttacgacgccctgcacatgcaggcactcccccccaggtga

SEQ ID NO: 40 - DNA, 1206 bp, synthetic construct
  misc_feature 1..1206: note = nucleotide sequence encoding an inducible Caspase 9
  source 1..1206: mol_type = other DNA
atgctcgagggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagc

SEQ ID NO: 41 - DNA, 54 bp, synthetic construct
  misc_feature 1..54: note = nucleotide sequence encoding 2A self-cleaving peptides T2A
  source 1..54: mol_type = other DNA
gagggcaggggaagtcttctaacatgcggggacgtggaggaaaatcccgggccc

SEQ ID NO: 42 - DNA, 63 bp, synthetic construct
  misc_feature 1..63: note = nucleotide sequence encoding a Signal peptide
  source 1..63: mol_type = other DNA
atggagtttgggctctcctggctcttcctggtcgcgattctgaagggggtccagtgttcacga

SEQ ID NO: 43 - DNA, 24 bp, synthetic construct
  misc_feature 1..24: note = nucleotide sequence encoding a flex linker
  source 1..24: mol_type = other DNA
ggcggaggttcaggcggaggaggg

SEQ ID NO: 44 - DNA, 48 bp, synthetic construct
  misc_feature 1..48: note = nucleotide sequence encoding DELTA CD34
  source 1..48: mol_type = other DNA
gaacttcctactcaggggactttctcaaacgttagcacaaacgtaagt

SEQ ID NO: 45 - DNA, 120 bp, synthetic construct
  misc_feature 1..120: note = nucleotide sequence encoding Hinge Spacer-CD8 alpha
  source 1..120: mol_type = other DNA
cccgccccaagaccccccacacctgcgccgaccattgcttctcaacccctgagtttgagacccgaggcctgccggccagctgccggcggggccgtgcatacaagaggactcgatttcgct

SEQ ID NO: 46 - DNA, 69 bp, synthetic construct
  misc_feature 1..69: note = CD8aTM sequence
  source 1..69: mol_type = other DNA
tgcgacatctacatctgggctcccctcgctggcacctgtggggtgctgctgctgtcactcgtgatcacc

SEQ ID NO: 47 - DNA, 21 bp, synthetic construct
  misc_feature 1..21: note = CD8a cytoplasmic sequence
  source 1..21: mol_type = other DNA
ctttattgcaaccatcgaaac

SEQ ID NO: 48 - DNA, 123 bp, synthetic construct
  misc_feature 1..123: note = CD28 cytoplasmic sequence
  source 1..123: mol_type = other DNA
agaagtaaacggtcaaggcttctgcacagcgattatatgaatatgacaccaagaagacctggtccaacccggaaacactatcagccctacgcgccccctagagacttcgcagcataccgctct

SEQ ID NO: 49 - DNA, 126 bp, synthetic construct
  misc_feature 1..126: note = 4-1BB sequence
  source 1..126: mol_type = other DNA
aagagagggagaaaaaaattgctctatatttttaaacaaccatttatgaggcccgtacagacaactcaggaagaggatggctgtagttgccgcttcccagaggaggaggaaggaggctgcgagttg

SEQ ID NO: 50 - DNA, 339 bp, synthetic construct
  misc_feature 1..339: note = CD3-Zeta chain
  source 1..339: mol_type = other DNA
agagttaaattcagtagaagtgcggatgcgcctgcttaccagcagggccagaaccaactgtacaatgaactgaatctcgggcgccgagaagagtatgacgtcctcgataagcggaggggtagggatcctgaaatgggtgggaagccaagaagaaaaaacccccaggaaggactgtataacgaacttcagaaggacaagatggcagaggcctactctgagattggcatgaaaggcgaacgacggcgcggtaaaggtcatgacgggctgtaccagggcctgtccacagcgacgaaggacacttacgacgccctgcacatgcaggcactcccccccaggtga

SEQ ID NO: 51 - DNA, 108 bp, synthetic construct
  misc_feature 1..108: note = OX40 sequence
  source 1..108: mol_type = other DNA
cgcgatcaaagactcccgcccgatgcccacaaaccccctggcgggggcagctttaggacacccattcaagaagagcaggcagacgcccacagcaccttggccaaaatt

SEQ ID NO: 52 - protein, 402 aa, synthetic construct
  REGION 1..402: note = inducible Caspase 9 sequence
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 53 - protein, 18 aa, synthetic construct
  REGION 1..18: note = 2A self-cleaving peptides T2A
EGRGSLLTCGDVEENPGP

SEQ ID NO: 54 - protein, 9 aa, synthetic construct
  REGION 1..9: note = linker
SGGGGSGGG

SEQ ID NO: 55 - protein, 11 aa, synthetic construct
  REGION 1..11: note = linker
SGGGGSGGGGS

SEQ ID NO: 56 - protein, 12 aa, synthetic construct
  REGION 1..12: note = linker
SGGGGSGGGGSG

SEQ ID NO: 57 - protein, 14 aa, synthetic construct
  REGION 1..14: note = linker
SGGGGSGGGGSGGG

SEQ ID NO: 58 - protein, 10 aa, synthetic construct
  REGION 1..10: note = linker
SGGGGSGGGG

SEQ ID NO: 59 - protein, 13 aa, synthetic construct
  REGION 1..13: note = linker
SGGGGSGGGGSGG